(19) United States
(12) Patent Application Publication
(10) Pub. No.: US 2024/0082402 A1
(43) Pub. Date: Mar. 14, 2024

(54) BISPECIFIC CHIMERIC ANTIGEN RECEPTORS BINDING TO CD19 AND CD22

(71) Applicant: Elpis Biopharmaceuticals, Lexington, MA (US)

(72) Inventors: Yan CHEN, Lexington, MA (US); Jenna NGUYEN, Lexington, MA (US); Kehao ZHAO, Lexington, MA (US); Keming ZHANG, Lexington, MA (US)

(73) Assignee: Elpis Biopharmaceuticals, Lexington, MA (US)

(21) Appl. No.: 18/262,496

(22) PCT Filed: Jan. 21, 2022
(86) PCT No.: PCT/US2022/013231

§ 371 Date: Jul. 21, 2023

Related U.S. Application Data
(60) Provisional application No. 63/140,752, filed on Jan. 22, 2021.

(51) Int. Cl. A61K 39/00; A61P 35/00; C07K 14/71; C07K 14/725; C07K 16/28; C12N 5/0783; C12N 15/86

(52) U.S. Cl. CPC A61K 39/464413; A61K 39/4611; A61K 39/4613; A61K 39/4614; A61K 39/4631; A61K 39/464412; A61P 35/00; C07K 14/7051; C07K 14/71; C07K 16/2803; C12N 5/0636; C12N 15/86; C07K 2317/31; C07K 2317/622; C07K 2319/02; C12N 2740/15043

(57) ABSTRACT

Bi-specific chimeric antigen receptors (CARs) capable of binding to both CD19 and CD22 and immune cells expressing such. Also provided herein are therapeutic uses of such immune cells (e.g., CAR-T cells) for eliminating disease cells such as cancer cells.

### CROSS REFERENCE TO RELATED APPLICATIONS

This application claims the benefit of the filing date of U.S. Provisional Application No. 63/140,752, filed Jan. 22, 2021, the entire contents of which are incorporated by reference herein.

### BACKGROUND OF THE INVENTION

Chimeric antigen receptor (CAR-T) T cells are genetically engineered T cells expressing an artificial T cell receptor for use in immunotherapy. The artificial T cell receptor (known as chimeric antigen receptor) can specifically bind disease cell antigens, such as cancer antigens. Upon binding to the disease cell, the CAR-T cells would be activated and eliminate the disease cell.

While CAR-T cell therapy have demonstrated efficacy in treatment of a few blood cancer, efficacy of the treatment may be affected by various factors, for example, tumor antigen escape, for example, the expression level of the tumor antigen may reduce to a level that CAR-T cells cannot engage and mediate cytotoxic activity. In some instances, tumor cells may escape killing by expressing an alternative form of the target antigen that lacks the binding epitope to the CAR. In other instances, tumor cells may escape killing by switching to a genetically related but phenotypically different disease (so called lineage switch).

Accordingly, it is of great interest to develop improved CAR-T approaches to address such challenges.

### SUMMARY OF THE INVENTION

The present disclosure is based, at least in part, on the development of anti-CD19/CD22 bispecific chimeric antigen receptors (CARs) having superior antigen binding affinity and specificity and superior anti-tumor effects as observed in an animal model. Accordingly, provided herein are anti-CD19/CD22 bispecific CARs, nucleic acid encoding such, host cells such as immune cells expressing the bispecific CAR, and therapeutic applications thereof.

In some aspects, the present disclosure features a bi-specific chimeric antigen receptor (CAR) specific to CD19 and CD22, comprising a first antigen binding moiety specific to CD19, a second antigen binding moiety to CD22, a co-stimulatory signaling domain, and a cytoplasmic signaling domain. The first antigen binding moiety may comprise the same heavy chain complementary determining regions (CDRs) and/or the same light chain CDRs as reference antibody EPC-001-1, which binds CD19. The second antigen binding moiety may comprise the same heavy chain CDRs and/or the same light chain CDRs as reference antibody EPC-001-2, EPC-001-3, or EPC-001-4, each of which binds CD22.

In some embodiments, the first antigen binding moiety may comprise the same heavy chain variable region (V_{H}) and the same light chain variable region (V_{L}) as the reference antibody EPC-001-1. Alternatively, or in addition, the second antigen binding moiety comprises the same heavy chain variable region (V_{H}) and the same light chain variable region (V_{L}) as the reference antibody EPC-001-2, EPC-001-2, or EPC-001-3.

In some embodiments, the first antigen binding moiety, the second antibody binding moiety, or both can be single-chain variable fragments (scFvs). For example, the first antigen binding moiety is a scFv comprising the amino acid sequence of SEQ ID NO: 9. Alternatively, or in addition, the second antigen binding moiety is a scFv comprising the amino acid sequence of SEQ ID NO: 18, 27, or 36.

In any of the CAR constructs disclosed herein, the co-stimulatory signaling domain can be from a co-stimulatory molecule selected from the CD28, 4-1BB, OX40, ICOS, CD27, CD40, or CD40L. In some embodiments, the cytoplasmic signaling domain is from CD3ζ. Any of the CAR disclosed herein may further comprising a hinge domain and a transmembrane domain In some instances, the hinge and transmembrane domains may be located the antigen binding moieties and the co-stimulatory signaling domain.

In some examples, the bi-specific CAR comprises a fusion polypeptide comprising, from N-terminus to C-terminus, (i) the first antigen binding moiety, (ii) the second antigen binding moiety, (iii) the co-stimulatory signaling domain, and (iv) the cytoplasmic signaling domain In other examples, the bi-specific CAR comprises a fusion polypeptide comprising, from N-terminus to C-terminus, (i) the second antigen binding moiety, (ii) the first antigen binding moiety, (iii) the co-stimulatory signaling domain, and (iv) the cytoplasmic signaling domain

In some instances, the bi-specific CAR may further comprise a peptide linker connecting the first antigen binding moiety and the second antigen binding moiety. Exemplary peptide linkers include, but are not limited to, GGGGS (SEQ ID NO:38), GGGGSGGGGS (SEQ ID NO:39), GGGGSGGGGSGGGGS (SEQ ID NO:40), or GSTSGSGKPGSGEGSTKG (SEQ ID NO:41).

In some embodiments, the bi-specific CAR disclosed here may comprise the amino acid sequence of any one of SEQ ID NOs: 48-53. In some examples, the bi-specific CAR disclosed herein may comprise the amino acid sequence of any one of SEQ ID NOs.: 55-60 and 63-66.

In another aspect, provided herein is a nucleic acid or a set of nucleic acid, which collectively encode any of the bi-specific CARs disclosed herein. For example, the nucleic acid may comprise a nucleotide sequence encoding a CAR comprising an amino acid sequence of any one of SEQ ID NOs: 48-53. In specific examples, the nucleic acid may comprise a nucleotide sequence encoding a CAR comprising an amino acid sequence of any one of SEQ ID NOs: 55-60 and 63-67.

In some instances, the nucleic acid or the set of nucleic acids may further comprises (i) a nucleotide sequence encoding a truncated epithelium growth factor receptor (EGFR) domain, which may comprise an extracellular domain and a transmembrane domain of an EGFR receptor, and (ii) a nucleotide sequence encoding a self-cleaving peptide, which is located between the nucleotide sequence encoding the bi-specific CAR and the nucleotide sequence encoding the truncated EGFR domain. In some examples, the truncated EGFR domain comprises the amino acid sequence of SEQ ID NO:68.

Any of the nucleic acid or set of nucleic acid can be an expression vector(s). In some examples, the expression vector(s) may be a viral vector(s).

In yet another aspect, the present disclosure features a genetically engineered immune cell, which expresses any of the bi-specific CARs disclosed herein. Such a genetically engineered immune cell may comprise any of the nucleic acids encoding the bi-specific CAR as disclosed herein. In some examples, the genetically engineered immune cell is a T cell. In some examples, the genetically engineered immune cell is an NK cell. In other examples, the genetically engineered immune cell may be a macrophage.

Also within the scope of the present disclosure are anti-CD19 CAR and anti-CD22 CAR, nucleic acids encoding such, and genetically engineered immune cells (e.g., T cells) expressing such.

In some embodiments, the anti-CD19 CAR may comprise an extracellular antigen binding domain that binds CD19, a co-stimulatory signaling domain, and a cytoplasmic signaling domain. The extracellular antigen binding domain may be an anti-CD19 single chain variable fragment (scFv) comprising the same heavy chain complementary determining regions (CDRs) and the same light chain CDRs as anti-CD19 antibody EPC-001-1. In some examples, the anti-CD19 scFv comprises the same heavy chain variable domain and the same light chain variable domain as anti-CD19 antibody EPC-001-1. In one example, the anti-CD19 scFv comprises the amino acid sequence of SEQ ID NO: 9. In one specific example, the anti-CD19 CAR may comprise the amino acid sequence of SEQ ID NO: 62.

In some embodiments, the anti-CD22 chimeric antigen receptor (CAR) may comprise an extracellular antigen binding domain that binds CD22, a co-stimulatory signaling domain, and a cytoplasmic signaling domain. In some examples, the extracellular antigen binding domain can be an anti-CD22 single chain variable fragment (scFv) comprising the same heavy chain complementary determining regions (CDRs) and/or the same light chain CDRs as anti-CD22 antibody EPC-001-2. For example, the anti-CD22 scFv may comprises the same heavy chain variable domain and/or the same light chain variable domain as anti-CD22 antibody EPC-001-2. In some examples, the extracellular antigen binding domain can be an anti-CD22 single chain variable fragment (scFv) comprising the same heavy chain complementary determining regions (CDRs) and/or the same light chain CDRs as anti-CD22 antibody EPC-001-3. For example, the anti-CD22 scFv may comprises the same heavy chain variable domain and/or the same light chain variable domain as anti-CD22 antibody EPC-001-3. In some examples, the extracellular antigen binding domain can be an anti-CD22 single chain variable fragment (scFv) comprising the same heavy chain complementary determining regions (CDRs) and/or the same light chain CDRs as anti-CD22 antibody or EPC-001-4. For example, the anti-CD22 scFv may comprises the same heavy chain variable domain and/or the same light chain variable domain as anti-CD22 antibody EPC-001-4. In some examples, the anti-CD22 scFv comprises the amino acid sequence of SEQ ID NO: 18, 27, or 36. In specific examples, the anti-CD22 CAR may comprise the amino acid sequence of SEQ ID NO: 61.

In addition, the present disclosure features a method for eliminating undesired cells in a subject, the method comprising administering to a subject in need thereof an effective amount of the genetically engineered immune cell disclosed herein, which expresses an anti-CD19 CAR, an anti-CD22 CAR, or an anti-CD19/CD22 CAR as those disclosed herein, or a pharmaceutical composition comprising such. In some instances, the undesired cells are cancer cells.

In some embodiments, the subject is a human cancer patient. For example, the subject can be a human cancer patient comprise CD19⁺ and/or CD22⁺ cancer cells. In some instances, the human cancer patient may have a hematopoietic malignancy, for example, a T cell malignancy or a B cell malignancy.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing the quantification of the surface expression of recombinant or endogenous CD19, CD22, or both on K562 cells, Raji cells, and Nalm6 cells by qFACS.

FIGS. 2A-2B include diagrams showing schematic designs of expression cassettes for expressing scFv antibodies and bi-specific chimeric antigen receptors (CARs). FIG. 2A is a diagram showing exemplary designs of the bispecific antibodies. FIG. 2B is a diagram illustrating exemplary designs of anti-CD19/CD22 bispecific chimeric antigen receptors (CARs).

FIG. 3 is a diagram showing binding activity of various bispecific antibodies as indicated to CD19⁺ and/or CD22⁺ cells, including K562 cells (CD19⁻ and CD22⁻), K562 cells engineered to express CD19 (CD19 K562), K562 cells engineered to express CD22 (CD22 K562), K562 cells engineered to express both CD19 and CD22 (CD19/CD22 K562), Raji cells (CD19⁺ and CD22+), and Nalm6 cells (CD19⁺ and CD22+).

FIGS. 4A-4B include photos showing expression of an exemplary anti-CD19/CD22 bispecific chimeric antigen receptor (CAR), EPC-001-19, in immune cells as detected by fluorescent dye Alexa Fluor® 647 labeled anti-EGFR antibody (EGFR is co-expressed with the bispecific CAR) or fluorescent dye Alexa Fluor® 647 labeled CD22-Fc fusion polypeptide. FIG. 4A: detected by anti-EGFR-AF647. FIG. 4B: detected by anti-human CD22-AF647.

FIGS. 5A-5D include diagrams showing Cytotoxic T Lymphocyte (CTL) activity of immune cells expressing an anti-CD19/CD22 bispecific CAR. FIG. 5A: percentage of killing (E:T=1:1). Left panel: EPC-001-11; Middle panel: EPC-001-12; Right panel: EPC-001-13. FIG. 5B: percentage of killing (E:T=1:1). Left panel: EPC-001-14; Middle panel: EPC-001-15; Right panel: EPC-001-16. FIG. 5C: Interferon γ (IFNγ) secretion (E:T=1:1). Left panel: EPC-001-11; Middle panel: EPC-001-12; Right panel: EPC-001-13. FIG. 5D: Interferon γ (IFNγ) secretion (E:T=1:1).). Left panel: EPC-001-14; Middle panel: EPC-001-15; Right panel: EPC-001-16.

FIGS. 6A-6C include diagrams showing CTL activity of T cells expressing an exemplary anti-CD19/CD22 bispecific CAR against target cells at various Effector-to-Target Cell (E:T) ratios. FIG. 6A: charts showing levels of specific cell lysis of CAR-T cells prepared from Donor 1 PBMCs against K562 cells, CD22 K562 cells, CD19 K562 cells, and CD19/CD22 K562 cells at different E:T ratios as indicated. FIG. 6B: charts showing CTL activity of CAR-T cells prepared from Donor 2 and Donor 3 PBMCs against K562 cells, CD22 K562 cells, CD19 K562 cells, and CD19/CD22 K562 cells at different E:T ratios as indicated. 5 FIG. 6C: charts showing IFNy levels in co-cultures of the CAR-T cells prepared from Donor 2 and Donor 3 PBMCs against K562 cells, CD22 K562 cells, CD19 K562 cells, and CD19/CD22 K562 cells at different E:T ratios as indicated.

FIG. 7 includes charts showing CTL time courses of T cells expressing various anti-CD19/CD22 bispecific CAR or anti-CD19, anti-CD22 monospecific CARs against K562 cells, CD22 K562 cells, CD19 K562 cells, and CD19/CD22 K562 cells at different E:T ratios as indicated.

FIG. 8 includes charts showing CTL activities of T cells expressing anti-CD19/CD22 bispecific CARs having different linkers connecting the anti-CD19 and anti-CD22 binding moieties in the CAR constructs.

FIG. 9 includes diagrams showing CAR-T cell proliferation upon engagement with target cells (E:T=1:1).

FIGS. 10A-10B include diagrams showing in vitro persistence of CAR-T cells upon multiple rounds of target cell challenge. FIG. 10A: percentage of killing (E:T=1:1). Left panel: target cell challenge 1 at 48 hr; Middle panel: target cell rechallenge 2 at 120 hr; Right pane: target cell rechallenge 3 at 192 hr. FIG. 10B: IFNγ secretion (E:T=1:1). Left panel: target cell challenge 1 at 48 hr; Middle panel: target cell rechallenge 2 at 120 hr; Right pane: target cell rechallenge 3 at 192 hr.

FIGS. 11A-11E show in vivo anti-tumor effects of T cells expressing anti-CD19/CD22 bispecific CAR in a mouse cancer model. FIG. 11A: images showing cancer cell luciferase in control mice (untreated) and mice treated with CAR-T cells at 0.125×10⁶ and 0.25×10⁶ cells. FIG. 11B: a chart showing inhibition of tumor growth in mice treated with the CAR-T cells by quantification of luciferase in tumor cells over the treatment course. FIGS. 11C-11D: charts showing tumor cell luciferase quantification on Day 14 and Day 33, respectively, after treatment. FIG. 11E: survival curves of control mice and mice treated with the CAR-T cells.

FIGS. 12A-12E include diagrams showing CAR-T cell expansion, phenotype and persistence in vivo. FIG. 12A: charts showing CAR-T cell counts and phenotype on Day 10, Day 19, and Day 33 after treatment. Top panel: Group 2 mice treated with 0.125×10⁶ CAR-T cells; Bottom panel: Group 3 mice treated with 0.25 ×10⁶ CAR-T cells. FIGS. 12B-12C: Day 33/Day 19 ratios of subtype of T cells in Group 2 and Group 3 mice, respectively. FIGS. 12D-12E: cell counts in spleen on Day 33 of Group 2 and Group 3 mice, respectively.

FIGS. 13A-13B show ELISA binding assays for anti-CD19 or CD22 scFv-Fc. FIG. 13A: Binding of anti-CD22 scFv-Fc, anti-19 scFv-Fc and anti-CD19/CD22 scFv-Fc to CD19. FIG. 13B: Binding of anti-CD22 scFv-Fc, anti-19 scFv-Fc and anti-CD19/CD22 scFv-Fc to CD22.

FIGS. 14A-14B show oxygen consumption of EPC-001-023 transduced T cells. FIG. 14A: Oxygen consumption in EPC-001-023 CAR T cells at day 5. FIG. 14B: Oxygen consumption in EPC-001-023 CART cells at day 11.

FIG. 15 shows western blot analysis of CD19 and CD22 protein in CD19 and CD22 knock out cells and parental Raji cells.

FIGS. 16A-16B show a quantitative FACS analysis of surface receptor expression. FIG. 16A: CD19 surface receptor count in the indicated cell lines. FIG. 16B: CD22 surface receptor count in the indicated cell lines.

FIG. 17 shows in vivo anti-tumor effects of EPC-001-23 CAR-T cells in an animal model implanted with parental and knockout Raji cells.

FIGS. 18A-18C show a quantitative assessment of the tumor load as determined by luciferase bioluminescence. FIG. 18A: Luciferase signal in parental Raji cells. FIG. 18B: Luciferase signal in CD22 knockout Raji cells. FIG. 18C: Luciferase signal in CD19 knockout Raji cells.

FIGS. 19A-19B show phenotyping analysis of EPC-001-23 CAR-T cells performed in PBMC and spleen samples collected at day 36. FIG. 19A: Phenotype analysis for CD3+ CAR+ cells in PBMC. FIG. 19B: Phenotype analysis for CD3⁺ CAR+ cells in the spleen.

FIGS. 20A-20C show phenotyping analysis of EPC-001-23 CAR-T cells performed in PBMC and spleen samples collected at day 36. FIG. 20A: CD3+ CAR+ T cell count in PBMC. FIG. 20B: Amounts of PD1+, Tim3− CD3⁺ CAR+ expression in PBMC. FIG. 20C: Amounts of PD1+, Tim3− CD3+ CAR+ expression in spleens.

FIGS. 21A-21C include diagrams showing EPC-001-23 bi-specific CAR-T cell expansion and activation in vitro as compared with the tisagenlecleucel control. FIG. 21A: T cell expansion as indicated by CAR+T cell counts; FIG. 21B: T cell activation as indicated by granzyme B+CAR-T cells at 72 hours. FIG. 21C: CAR-T cell cytotoxicity.

FIG. 22A-22B include diagrams showing in vivo cytotoxicity of EPC-001-23 bi-specific CAR-T cell in mice engrafted with Raji cells or CD22KO Raji cells as compared with the tisagenlecleucel control. FIG. 22A: a photo showing tumor imaging in mice treated with CAR-T cells as indicated. FIG. 22B: a chart showing CAR-T cell expansion and persistence in vivo.

FIG. 23A-23B include diagrams showing in vivo cytotoxicity of EPC-001-23 bi-specific CAR-T cell in mice engrafted with Raji cells or CD19KO Raji cells as compared with the tisagenlecleucel control. FIG. 23A: a photo showing tumor imaging in mice treated with CAR-T cells as indicated. FIG. 23B: a chart showing CAR-T cell expansion and persistence in vivo.

### DETAILED DESCRIPTION OF THE INVENTION

B-lymphocyte antigen CD19 is a member of the immunoglobulin super family expressed primarily on B lineage cells and follicular dendritic cells. It has been reported that CD19 acts as an adaptor protein to recruit cytoplasmic signaling proteins and as a modulator (via the CD19/CD21 complex) to decrease the threshold for the signaling pathway meditated by B cell receptors.

Cluster of differentiation 22 (CD22) is a member of the SIGLEC family of lectins. This molecule expresses at a high level on the surface of mature B cells as relative to immature B-cells. As an inhibitory receptor for B cell receptor (BCR) signaling, it plays a regulatory role in preventing over-activation of the immune system.

Both CD19 and CD22 have been established as promising targets for treatment of certain diseases, such as leukemia. However, the effectiveness of a treatment targeting only CD19 or only CD22 may be affected due to, for example, tumor antigen escape, leading to reduced treatment efficacy.

Provided herein are bispecific chimeric antigen receptors (CARs) capable of binding to both CD19 and CD22 and genetically engineered immune cells expressing such bispecific CARs. The anti-CD19/CD22 bispecific CARs disclosed herein showed superior binding activity to both surface-expressing CD19 and CD22 and superior cytotoxic T lymphocyte-mediated cytotoxicity. Further, the anti-CD19/CD22 bispecific CARs showed superior in vivo anti-tumor activity as observed in a mouse model. Moreover, the bi-specific CAR-T cells were shown to be effective in killing cancer cells that are either CD19/CD22 double positive or express only one of the two target antigens, indicating that the bi-specific Car-T cells would maintain treatment efficacy in the context of either CD19 escape or CD22 escape.

Accordingly, immune cells expressing the bispecific CARs disclosed herein would be expected to exert superior therapeutic effects in treating diseases involving CD19±/CD22⁺, CD19⁻/CD22⁺ or CD19⁺/CD22⁻ disease cells (e.g., cancer cells) and addressing issues such as tumor antigen escape associated with monospecific CAR-T therapy or targeted therapy. CD19/CD22 bispecific tandem CAR has bivalent target engagement and therefore can lock CD19 and CD22 targets and prevent or delay target escape and potentially lower dose. In addition, each CD19 or CD22 binding domain can independently engage CD19 or CD22 expressing cells and mediate cancer cell killing when either target escapes.

Accordingly, the present disclosure features anti-CD19/CD22 bispecific CARs, nucleic acids encoding such, host cells such as immune cells (e.g., T cells, NK cells, or macrophages) expressing the bispecific CARs, and therapeutic uses of such immune cells in treating diseases associated with CD19⁺ and/or CD22⁺ disease cells.

### I. Chimeric Antigen Receptors

As used herein, the term “chimeric antigen receptor” or “CAR” refers to an artificial immune cell receptor that is capable of binding to an antigen expressed by undesired cells, for example, a tumor associated antigen (TAA) (e.g., CD19 or CD22). Generally, a CAR may comprise a fusion polypeptide, which comprises an extracellular antigen binding domain (e.g., a single chain variable fragment or scFv derived from an antibody specific to the target antigen), a co-stimulatory domain, and an intracellular signaling domain. In some instances, the fusion polypeptide may further comprise a hinge and transmembrane domain located at the C-terminus of the extracellular antigen binding domain In some embodiments, the CARs disclosed herein are T cell receptors. In other embodiments, the CARs disclosed herein may be NK cell receptors.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an antibody moiety disclosed herein may share the same heavy chain and/or light chain complementary determining regions (CDRs) or the same V_{H} and/or V_{L} chains as a reference antibody. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-CD19 antibodies may have the same V_{H}, the same V_{L}, or both as compared to an exemplary antibody described herein.

In some embodiments, an antibody moiety disclosed herein may share a certain level of sequence identity as compared with a reference sequence. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, an antibody moiety disclosed herein may have one or more amino acid variations relative to a reference antibody. The amino acid residue variations as disclosed in the present disclosure (e.g., in framework regions and/or in CDRs) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The anti-CD19/CD22 bispecific CARs disclosed here each comprises an anti-CD19 moiety and an anti-CD22 moiety in the extracellular antigen binding domain.
(a) Anti-CD19 Binding Moiety

The anti-CD19 binding moiety in any of the CARs disclosed herein (e.g., any of the anti-CD19/CD22 bispecific CARs disclosed herein) may be in an scFv format, which is a fusion polypeptide comprising the heavy chain variable domain (V_{H}) and the light chain variable domain (V_{L}) of an anti-CD19 antibody connected by a peptide linker. In the scFv fragment, the V_{H} and V_{L} fragments may be in any orientation. In some instances, the scFv may comprise, from the N-terminus to the C-terminus, a V_{L} fragment, a peptide linker, and a V_{H} fragment. Alternatively, the scFv may comprise, from the N-terminus to the C-terminus, a V_{H} fragment, a peptide linker, and a V_{L} fragment. In some examples, a scFv may further comprise an N-terminal signal peptide for directing the CAR comprising the scFv to cell surface.

In some embodiments, the anti-CD19 binding moiety may be derived from anti-CD19 antibody EPC-001-1 (see Table 1 below). The heavy chain and light chain complementary determining regions provided in Table 1 are based on Kabat definition. See also PCT/US2020/047035, filed on August 19, 2020, the relevant disclosures of which are incorporated by reference for the subject matter and purposed referenced herein.

TABLE 1
Anti-CD19 Antibody

| Antibody | | Amino Acid Sequence | SEQ ID |
|---|---|---|---|
| EPC-001-1 | V_{H} CDR1 | GYYWT | 1 |
| (anti-CD19) | V_{H} CDR2 | EINHGGSSNYNPSLKS | 2 |
| | V_{H} CDR3 | GLGYRSGWYEVENAFDI | 3 |
| | V_{H} | QVQLQQWGAGLLKPSETLSLTCAVYGGSFSGYYWTWIRQPPGKGL | 4 |
| | | EWIGEINHGGSSNYNPSLKSRVTISVDTSKKQFSLNLNSVTAADT | |
| | | AVYYCARGLGYRSGWYEVENAFDIWGQGTMVTVSS | |
| | V_{L} CDR1 | GGNKIESRSVH | 5 |
| | V_{L} CDR2 | DDGARPS | 6 |
| | V_{L} CDR3 | QVWDGSSVI | 7 |
| | V_{L} | QPVLTQPPSVSVAPGQTARITCGGNKIESRSVHWYQQKPGQAPVL | 8 |
| | | VVYDDGARPSGIPERLSGSNSGDTATLTISRVEPGDEADYYCQVW | |
| | | DGSSVIFGGGTKLTVL | |
| | scFv | QVQLQQWGAGLLKPSETLSLTCAVYGGSFSGYYWTWIRQPPGKGL | 9 |
| | | EWIGEINHGGSSNYNPSLKSRVTISVDTSKKQFSLNLNSVTAADT | |
| | | AVYYCARGLGYRSGWYEVENAFDIWGQGTMVTVSSGGGGSGGGGS | |
| | | GGGGSQPVLTQPPSVSVAPGQTARITCGGNKIESRSVHWYQQKPG | |
| | | QAPVLVVYDDGARPSGIPERLSGSNSGDTATLTISRVEPGDEADY | |
| | | YCQVWDGSSVIFGGGTKLTVL | |

An anti-CD19 binding moiety (and an anti-CD22 binding moiety disclosed below) derived from a reference antibody refers to binding moieties having substantially similar structural and functional features as the reference antibody. Structurally, the binding moiety may have the same heavy and/or light chain complementary determining regions or the same V_{H} and/or V_{L} chains as the reference antibody. Alternatively, the binding moiety may only have a limited number of amino acid variations in one or more of the framework regions and/or in one or more of the CDRs without significantly affecting its binding affinity and binding specificity relative to the reference antibody. See descriptions below.

In some examples, the anti-CD19 binding moiety may comprise the same heavy chain CDRs as those in antibody EPC-001-1, which are provided in Table 1 above. Alternatively, or in addition, the anti-CD19 binding moiety may have the same light chain CDRs as those in antibody EPC-001-1, which are also provided in Table 1 above. Such an anti-CD19 binding moiety may comprise the same V_{H} and/or V_{L} chains as EPC-001-1. Alternatively, the anti-CD19 binding moiety may comprise amino acid variations in one or more of the framework regions relative to the corresponding framework regions in EPC-001-1. For example, the anti-CD19 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in EPC-001-1.

In some embodiments, the anti-CD19 moiety may comprise a certain level of variations in one or more of the CDRs relative to those of EPC-001-1. For example, the anti-CD19 moiety may comprise heavy chain CDRs that are at least 80% (e.g., 85%, 90%, 95%, or 98%) sequence identity, individually or collectively, as compared with the V_{H} CDRs of EPC-001-1.

Alternatively, or in addition, the anti-CD19 antibody may comprise light chain CDRs that are at least 80% (e.g., 85%, 90%, 95%, or 98%) sequence identity, individually or collectively, as compared with the V_{L} CDRs as EPC-001-1. As used herein, “individually” means that one CDR of an antibody shares the indicated sequence identity relative to the corresponding CDR of a reference antibody (e.g., EPC-001-1 or any of the anti-CD22 reference antibodies disclosed below). “Collectively” means that three V_{H} or V_{L} CDRs of an antibody in combination share the indicated sequence identity relative the corresponding three V_{H} or V_{L} CDRs of the reference antibody in combination.

In some instances, the anti-CD19 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of EPC-001-1. In some instances, the anti-CD19 moiety may comprise the same heavy chain CDR3 as the heavy chain CDR3 of EPC-001-1 and comprise one or more amino acid variations in one or more of the other heavy chain and light chain CDRs.

In some examples, the anti-CD19 moiety disclosed herein may comprise the amino acid sequence of SEQ ID NO: 9. Alternatively, the anti-CD19 moiety may comprise an amino acid sequence at least 85% (e.g., at least 90%, at least 95%, at least 98%, or above) identical to SEQ ID NO: 9. In other examples, the anti-CD19 moiety disclosed herein may comprise the same V_{H} and V_{L} sequences as in SEQ ID NO:9 but has a reversed orientation of the V_{H} and V_{L} fragments as in SEQ ID NO:9.

Any of the anti-CD19 moieties disclosed herein (e.g., SEQ ID NO: 9 or its counterpart having reversed V_{H} and V_{L} orientation) may be used for constructing the anti-CD19/CD22 bispecific CARs as disclosed herein.

### (b) Anti-CD22 Binding Moiety

The anti-CD22 binding moiety in any of the CARs disclosed herein (e.g., any of the anti-CD19/CD22 bispecific CARs disclosed herein) may be in an scFv format, which is a fusion polypeptide comprising the heavy chain variable domain (V_{H}) and the light chain variable domain (V_{L}) of an anti-CD22 antibody connected by a peptide linker. In the scFv fragment, the V_{H} and V_{L} fragments may be in any orientation. In some instances, the scFv may comprise, from the N-terminus to the C-terminus, a V_{L} fragment, a peptide linker, and a V_{H} fragment. Alternatively, the scFv may comprise, from the N-terminus to the C-terminus, a V_{H} fragment, a peptide linker, and a V_{L} fragment. In some examples, a scFv may further comprise an N-terminal signal peptide for directing the CAR comprising the scFv to cell surface.

In some embodiments, the anti-CD22 binding moiety may be derived from anti-CD22 antibody EPC-001-2, EPC-001-3, or EPC-001-4 (see Table 2 below). The heavy chain and light chain complementary determining regions provided in Table 1 are based on Kabat definition. See also PCT/US2020/047479, filed on Aug. 21, 2020, the relevant disclosures of which are incorporated by reference for the subject matter and purposed referenced herein.

TABLE 2
Anti-CD22 Antibodies

| Antibody | | Amino Acid Sequence | SEQ ID |
|---|---|---|---|
| EPC-001-2 | V_{H} CDR1 | SYGIS | 10 |
| (anti-CD22) | V_{H} CDR2 | WISAYNGNTNYAQKLQG | 11 |
| | V_{H} CDR3 | DPGIAVAGTVDY | 12 |
| | V_{H} | QVQLVQSGAEVKRPGASVKVSCKASGYTFTSYGISWVRQAPGQG | 13 |
| | | LEWMGWISAYNGNTNYAQKLQGRVTMTTDTSTSTAYMELRSLRS | |
| | | DDTAVYYCARDPGIAVAGTVDYWGQGTLVTVSS | |
| | V_{L} CDR1 | RASQSVSSNLA | 14 |
| | V_{L} CDR2 | GASIKAT | 15 |
| | V_{L} CDR3 | QQYHTWTPVT | 16 |
| | V_{L} | EIVMTQSPATLSVSPGEGVTLSCRASQSVSSNLAWYQQRPGQAP | 17 |
| | | RLLIYGASIKATDVPDRFSGGGSGTDFTLSISNVQSEDFAVYYC | |
| | | QQYHTWTPVTFGGGTKVEIK | |
| | scFv | QVQLVQSGAEVKRPGASVKVSCKASGYTFTSYGISWVRQAPGQG | 18 |
| | | LEWMGWISAYNGNTNYAQKLQGRVTMTTDTSTSTAYMELRSLRS | |
| | | DDTAVYYCARDPGIAVAGTVDYWGQGTLVTVSSGGGGSGGGGSG | |
| | | GGGSEIVMTQSPATLSVSPGEGVTLSCRASQSVSSNLAWYQQRP | |
| | | GQAPRLLIYGASIKATDVPDRFSGGGSGTDFTLSISNVQSEDFA | |
| | | VYYCQQYHTWTPVTFGGGTKVEIK | |
| EPC-001-3 | V_{H} CDR1 | SYGMH | 19 |
| (anti-CD22) | V_{H} CDR2 | VIWYDGSNKYYADSVKG | 20 |
| | V_{H} CDR3 | DGWTGFDY | 21 |
| | V_{H} | EVQLVQSGGGVVQPGKSLRLSCAASGFTFSSYGMHWVRQAPGKG | 22 |
| | | LEWVAVIWYDGSNKYYADSVKGRFTISRDNSKNTLYLQMNSLRA | |
| | | EDTAVYYCARDGWTGFDYWGQGTTVTVSS | |
| | V_{L} CDR1 | RASQSVSSNLA | 23 |
| | V_{L} CDR2 | GASIKAT | 24 |
| | V_{L} CDR3 | QQYHTWPPVT | 25 |
| | V_{L} | EIVLTQSPATLSVSPGEGVTLSCRASQSVSSNLAWYQQRPGQAP | 26 |
| | | RLLIYGASIKATDVPDRFSGGGSGTDFTLSISNLQSEDFAVYYC | |
| | | QQYHTWPPVTFGGGTKVEIK | |
| | scFv | EVQLVQSGGGVVQPGKSLRLSCAASGFTFSSYGMHWVRQAPGKG | 27 |
| | | LEWVAVIWYDGSNKYYADSVKGRFTISRDNSKNTLYLQMNSLRA | |
| | | EDTAVYYCARDGWTGFDYWGQGTTVTVSSGGGGSGGGGSGGGGS | |
| | | EIVLTQSPATLSVSPGEGVTLSCRASQSVSSNLAWYQQRPGQAP | |
| | | RLLIYGASIKATDVPDRFSGGGSGTDFTLSISNLQSEDFAVYYC | |
| | | QQYHTWPPVTFGGGTKVEIK | |
| EPC-001-4 | V_{H} CDR1 | SYGIS | 28 |
| (anti-CD22) | V_{H} CDR2 | WISAYNGNTNYAQKLQG | 29 |
| | V_{H} CDR3 | DYGDPSGDDY | 30 |
| | V_{H} | EVQLVQSGAEVKKPGASVKVSCKASGYTFTSYGISWVRQAPGQG | 31 |
| | | LEWMGWISAYNGNTNYAQKLQGRVTMTTDTSTSTAYMELRSLRS | |
| | | DDTAVYYCARDYGDPSGDDYWGQGTLVTVSS | |
| | V_{L} CDR1 | RASQSVSSNLA | 32 |
| | V_{L} CDR2 | GASIKAT | 33 |
| | V_{L} CDR3 | QQYHTWPPVT | 34 |
| | V_{L} | EIVLTQSPATLSVSPGEGVTLSCRASQSVSSNLAWYQQRPGQAP | 35 |
| | | RLLIYGASIKATDVPDRFSGGGSGTDFTLSISNLQSEDFAVYYC | |
| | | QQYHTWPPVTFGGGTKVEIK | |
| | scFv | EVQLVQSGAEVKKPGASVKVSCKASGYTFTSYGISWVRQAPGQG | 36 |
| | | LEWMGWISAYNGNTNYAQKLQGRVTMTTDTSTSTAYMELRSLRS | |
| | | DDTAVYYCARDYGDPSGDDYWGQGTLVTVSSGGGGSGGGGSGGG | |
| | | GSEIVLTQSPATLSVSPGEGVTLSCRASQSVSSNLAWYQQRPGQ | |
| | | APRLLIYGASIKATDVPDRFSGGGSGTDFTLSISNLQSEDFAVY | |
| | | YCQQYHTWPPVTFGGGTKVEIK | |

In some examples, the anti-CD22 binding moiety may comprise the same heavy chain CDRs as those in antibody EPC-001-2, which are provided in Table 2 above. Alternatively, or in addition, the anti-CD22 binding moiety may have the same light chain CDRs as those in antibody EPC-001-2, which are also provided in Table 2 above. Such an anti-CD22 binding moiety may comprise the same V_{H} and/or V_{L} chains as EPC-001-2. Alternatively, the anti-CD22 binding moiety may comprise amino acid variations in one or more of the framework regions relative to the corresponding framework regions in EPC-001-2. For example, the anti-CD22 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in EPC-001-2.

In some embodiments, the anti-CD22 moiety may comprise a certain level of variations in one or more of the CDRs relative to those of EPC-001-2. For example, the anti-CD22 moiety may comprise heavy chain CDRs that are at least 80% (e.g., 85%, 90%, 95%, or 98%) sequence identity, individually or collectively, as compared with the V_{H} CDRs of EPC-001-2. Alternatively, or in addition, the anti-CD22 antibody may comprise light chain CDRs that are at least 80% (e.g., 85%, 90%, 95%, or 98%) sequence identity, individually or collectively, as compared with the V_{L} CDRs as EPC-001-2.

In some instances, the anti-CD22 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of EPC-001-2. In some instances, the anti-CD22 moiety may comprise the same heavy chain CDR3 as the heavy chain CDR3 of EPC-001-2 and comprise one or more amino acid variations in one or more of the other heavy chain and light chain CDRs.

In some examples, the anti-CD22 moiety disclosed herein may comprise the amino acid sequence of SEQ ID NO: 18. Alternatively, the anti-CD22 moiety may comprise an amino acid sequence at least 85% (e.g., at least 90%, at least 95%, at least 98%, or above) identical to SEQ ID NO: 18. In other examples, the anti-CD22 moiety disclosed herein may comprise the same V_{H} and V_{L} sequences as in SEQ ID NO:18 but has a reversed orientation of the V_{H} and V_{L} fragments as in SEQ ID NO:18.

In some examples, the anti-CD22 binding moiety may comprise the same heavy chain CDRs as those in antibody EPC-001-3, which are provided in Table 2 above. Alternatively, or in addition, the anti-CD22 binding moiety may have the same light chain CDRs as those in antibody EPC-001-3, which are also provided in Table 2 above. Such an anti-CD22 binding moiety may comprise the same V_{H} and/or V_{L} chains as EPC-001-3. Alternatively, the anti-CD22 binding moiety may comprise amino acid variations in one or more of the framework regions relative to the corresponding framework regions in EPC-001-3. For example, the anti-CD22 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in EPC-001-3.

In some embodiments, the anti-CD22 moiety may comprise a certain level of variations in one or more of the CDRs relative to those of EPC-001-3. For example, the anti-CD22 moiety may comprise heavy chain CDRs that are at least 80% (e.g., 85%, 90%, 95%, or 98%) sequence identity, individually or collectively, as compared with the V_{H} CDRs of EPC-001-3. Alternatively, or in addition, the anti-CD22 antibody may comprise light chain CDRs that are at least 80% (e.g., 85%, 90%, 95%, or 98%) sequence identity, individually or collectively, as compared with the V_{L} CDRs as EPC-001-3.

In some instances, the anti-CD22 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of EPC-001-3. In some instances, the anti-CD22 moiety may comprise the same heavy chain CDR3 as the heavy chain CDR3 of EPC-001-3 and comprise one or more amino acid variations in one or more of the other heavy chain and light chain CDRs.

In some examples, the anti-CD22 moiety disclosed herein may comprise the amino acid sequence of SEQ ID NO: 27. Alternatively, the anti-CD22 moiety may comprise an amino acid sequence at least 85% (e.g., at least 90%, at least 95%, at least 98%, or above) identical to SEQ ID NO: 27. In other examples, the anti-CD22 moiety disclosed herein may comprise the same V_{H} and V_{L} sequences as in SEQ ID NO:27 but has a reversed orientation of the V_{H} and V_{L} fragments as in SEQ ID NO:27.

In some examples, the anti-CD22 binding moiety may comprise the same heavy chain CDRs as those in antibody EPC-001-4, which are provided in Table 2 above. Alternatively, or in addition, the anti-CD22 binding moiety may have the same light chain CDRs as those in antibody EPC-001-4, which are also provided in Table 2 above. Such an anti-CD22 binding moiety may comprise the same V_{H} and/or V_{L} chains as EPC-001-4. Alternatively, the anti-CD22 binding moiety may comprise amino acid variations in one or more of the framework regions relative to the corresponding framework regions in EPC-001-4. For example, the anti-CD22 binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in EPC-001-4.

In some embodiments, the anti-CD22 moiety may comprise a certain level of variations in one or more of the CDRs relative to those of EPC-001-4. For example, the anti-CD22 moiety may comprise heavy chain CDRs that are at least 80% (e.g., 85%, 90%, 95%, or 98%) sequence identity, individually or collectively, as compared with the V_{H} CDRs of EPC-001-4. Alternatively, or in addition, the anti-CD22 antibody may comprise light chain CDRs that are at least 80% (e.g., 85%, 90%, 95%, or 98%) sequence identity, individually or collectively, as compared with the V_{L} CDRs as EPC-001-4.

In some instances, the anti-CD22 moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of EPC-001-4. In some instances, the anti-CD22 moiety may comprise the same heavy chain CDR3 as the heavy chain CDR3 of EPC-001-4 and comprise one or more amino acid variations in one or more of the other heavy chain and light chain CDRs.

In some examples, the anti-CD22 moiety disclosed herein may comprise the amino acid sequence of SEQ ID NO: 36. Alternatively, the anti-CD22 moiety may comprise an amino acid sequence at least 85% (e.g., at least 90%, at least 95%, at least 98%, or above) identical to SEQ ID NO: 36. In other examples, the anti-CD22 moiety disclosed herein may comprise the same V_{H} and V_{L} sequences as in SEQ ID NO:36 but has a reversed orientation of the V_{H} and V_{L} fragments as in SEQ ID NO:36.

Any of the anti-CD22 moieties disclosed herein may be used for constructing the anti-CD19/CD22 bispecific CARs as disclosed herein. In some examples, the anti-CD22 moiety may comprise the amino acid sequence of SEQ ID NO: 18, or its counterpart having reversed V_{H} and V_{L} orientation. In some examples, the anti-CD22 moiety may comprise the amino acid sequence of SEQ ID NO: 27, or its counterpart having reversed V_{H} and V_{L} orientation. In some examples, the anti-CD22 moiety may comprise the amino acid sequence of SEQ ID NO: 36, or its counterpart having reversed V_{H} and V_{L} orientation.
(c) Other Components of Chimeric Antigen Receptor Constructs

In addition to the extracellular antigen binding domains disclosed herein, any of the CARs, including the anti-CD19/CD22 bispecific CARs, may further comprise one or more intracellular signaling domains (e.g., co-stimulatory and cytoplasmic signaling domains), and optionally a hinge domain, a transmembrane domain, an N-terminal signal peptide, or a combination thereof. In some instances, the CAR can be co-expressed with a suicide gene (e.g., a truncated EGFR gene) in a host immune cells. For example, the CAR coding sequence and the suicide gene may be configured in a bicistronic expression cassette, in which the CAR coding sequence and the suicide gene may be linked via a self-cleavage peptide (e.g., P2A or T2A) coding sequence. Examples are provided in Table 3 below.

TABLE 3
Amino Acid Sequences of Components in Chimeric Antigen Receptors or Co-
Expressed with Such

| Name | Amino Acid Sequence | SEQ ID |
|---|---|---|
| Signal Peptide | MLLLVTSLLLCELPHPAFLLIP | 37 |
| G₄S Linker | GGGGS | 38 |
| (G₄S)₂ Linker | GGGGSGGGGS | 39 |
| (G₄S)₃ Linker | GGGGSGGGGSGGGGS | 40 |
| Linker | GSTSGSGKPGSGEGSTKG | 41 |
| Hinge | ESKYGPPCPPCP | 42 |
| Transmembrane | MFWVLVVVGGVLACYSLLVTVAFIIFWV | 43 |
| (TM) | | |
| 4-1BB | KRGRKKLLYIFKQPFMRPVQTTQEEDGCSCRFPEEEEGGCEL | 44 |
| CD3z | RVKFSRSADAPAYQQGQNQLYNELNLGRREEYDVLDKRRGRDPEMGGKP | 45 |
| | RRKNPQEGLYNELQKDKMAEAYSEIGMKGERRRGKGHDGLYQGLSTATK | |
| | DTYDALHMQALPPR | |
| T2A | LEGGGEGRGSLLTCGDVEENPGPR | 46 |
| EGFRt | MLLLVTSLLLCELPHPAFLLIPRKVCNGIGIGEFKDSLSINATNIKHFK | 47 |
| | NCTSISGDLHILPVAFRGDSFTHTPPLDPQELDILKTVKEITGFLLIQA | |
| | WPENRTDLHAFENLEIIRGRTKQHGQFSLAVVSLNITSLGLRSLKEISD | |
| | GDVIISGNKNLCYANTINWKKLFGTSGQKTKIISNRGENSCKATGQVCH | |
| | ALCSPEGCWGPEPRDCVSCRNVSRGRECVDKCNLLEGEPREFVENSECI | |
| | QCHPECLPQAMNITCTGRGPDNCIQCAHYIDGPHCVKTCPAGVMGENNT | |
| | LVWKYADAGHVCHLCHPNCTYGCTGPGLEGCPTNGPKIPSIATGMVGAL | |
| | LLLLVVALGIGLFM | |
| | Signal peptide italicized | |

### Signaling Domains

Any of the CAR constructs disclosed herein, including anti-CD19/CD22 bispecific CARs, comprise one or more intracellular signaling domains, which typically contain a co-stimulatory domain and a cytoplasmic signaling domain. A “co-stimulatory signaling domain” refers to at least a fragment of a co-stimulatory signaling protein that mediates signal transduction within a cell to induce an immune response such as an effector function (a secondary signal). A cytoplasmic signaling domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells.

In some embodiments, the co-stimulatory signaling domain and the cytoplasmic signaling domain are for use in CAR constructs disclosed herein that are to be introduced into T cells. In some embodiments, the co-stimulatory signaling domain and the cytoplasmic signaling domain are for use in CAR constructs disclosed herein that are to be introduced into NK cells.

In some instances, a co-stimulatory signaling domain may be derived from a co-stimulatory protein involved in T cell responses, for example, a member of the B7/CD28 family, a member of the TNF superfamily, a member of the SLAM family, or any other co-stimulatory molecules. Examples include, but are not limited to, 4-1BB, CD28, OX40, ICOS, CD40, CD4OL, CD27, GITR, HVEM, TIM1, LFA1(CD11a) or CD2. In specific examples, the co-stimulatory signaling domain is a 4-1BB signaling domain (e.g., SEQ ID NO: 44 in Table 3 above).

The cytoplasmic signaling domain may comprise an immunoreceptor tyrosine-based activation motif (ITAM) domain or may be ITAM free. An “ITAM,” as used herein, is a conserved protein motif that is generally present in the tail portion of signaling molecules expressed in many immune cells. Exemplary cytoplasmic signaling domains include the signaling domain of CD3ζ, e.g., SEQ ID NO: 45.

In some instances, a co-stimulatory signaling domain may be derived from a co-stimulatory protein involved in NK cell responses. Examples include, but are not limited to, DAP10, DAP12, 2B4, NKG2D, FcRIy, NKp30, NKp44, or NKp46. Exemplary cytoplasmic signaling domains for use in NK cell CARs include, but are not limited to, CD3ζ, e.g., SEQ ID NO: 45.

### Hinge and Transmembrane Domains

In some instances, the CAR construct disclosed herein (e.g., any of the anti-CD19/CD22 bispecific CARs) may contain a transmembrane domain, which can be a hydrophobic alpha helix that spans the membrane. A “transmembrane domain” can be a peptide fragment that is thermodynamically stable in a cell membrane, preferably a eukaryotic cell membrane. The transmembrane domain can provide stability of the CAR containing such. Exemplary transmembrane domains may be a CD8 transmembrane domain, or a CD28 transmembrane domain. In one example, the transmembrane domain can comprise SEQ ID NO:43 shown in Table 3 above.

Alternatively, or in addition, the CAR construct disclosed herein may also comprise a hinge domain, which may be located between the extracellular antigen binding domain and the transmembrane domain, or between the transmembrane domain and the intracellular signaling domain A hinge domain may function to provide flexibility to the CAR, or domains thereof, or to prevent steric hindrance of the CAR, or domains thereof. A hinge domain may contain 5-20 amino acid residues. In some embodiments, the hinge domain may be a CD8 hinge domain or an IgG hinge. Other hinge domains may be used. In one example, the hinge domain can comprise SEQ ID NO:42 shown in Table 3 above.

### (d) Anti-CD19/CD22 Bispecific CARs

In some aspects, provided herein are anti-CD19/CD22 bispecific CAR comprising an anti-CD19 moiety (e.g., an anti-CD19 scFv such as those disclosed herein), an anti-CD22 moiety (e.g., an anti-CD22 scFv such as those disclosed), one or more intracellular signaling domains such as co-stimulatory signaling domains and cytoplasmic signaling domains, and optionally a hinge domain and a transmembrane domain as disclosed herein. In some instances, the anti-CD19/CD22 bispecific CAR may be a single polypeptide comprising both the anti-CD19 moiety and the anti-CD22 moiety. In other instances, the anti-CD19/CD22 bispecific 10 CAR may be a multiple-chain (e.g., 2-chain) molecule. The anti-CD19 moiety and the anti-CD22 moiety may be located on separate polypeptides.

In some embodiments, the anti-CD19/CD22 bispecific CAR disclosed herein may comprise an anti-CD19 binding moiety (e.g., scFv) derived from EPC-001-1 and an anti-CD22 binding moiety derived from EPC-001-2. In other embodiments, the anti-CD19/CD22 bispecific CAR disclosed herein may comprise an anti-CD19 binding moiety (e.g., scFv) derived from EPC-001-1 and an anti-CD22 binding moiety derived from EPC-001-3. In yet other embodiments, the anti-CD19/CD22 bispecific CAR disclosed herein may comprise an anti-CD19 binding moiety (e.g., scFv) derived from EPC-001-1 and an anti-CD22 binding moiety derived from any one of EPC-001-2-4.

Various combinations of anti-CD19 and anti-CD22 antibodies were investigated to make anti-CD19/CD22 bispecific cell engagement. Many of such bispecific scFvs either showed low expression levels or low binding activity to CD19 and/or CD22. Unexpectedly, it was found that anti-CD19/CD22 bispecific scFvs made from the anti-CD19 parent clone EPC-001-1 and 3 of the anti-CD22 parent clones, EPC-001-2, EPC-001-3, and EPC-001-4, showed desired levels of bispecific scFv expression and maintained strong and specific target cell engagement compared to monoclonal anti-CD19 or anti-CD22 scFvs.

The anti-CD19 binding moiety derived from EPC-001-1(e.g., scFv) may be any of the anti-CD19 moieties relating to EPC-001-1 disclosed above. In some instances, it may comprise the same heavy chain and/or light chain CDRs as EPC-001-1. In specific examples, the scFv may comprise the same V_{H} and/or same V_{L} as EPC-001-1. In some instances, the scFv may comprise, from the N-terminus to the C-terminus, a V_{L} fragment (e.g., SEQ ID NO:8), a peptide linker (e.g., any one of SEQ ID NOs: 38-41), and a V_{H} fragment (e.g., SEQ ID NO:4). Alternatively, the scFv may comprise, from the N-terminus to the C-terminus, a V_{H} fragment (e.g., SEQ ID NO:4), a peptide linker (e.g., any one of SEQ ID NOs: 38-41), and a V_{L} fragment (e.g., SEQ ID NO:8). In one specific example, the anti-CD19 moiety may comprise SEQ ID NO:9.

The anti-CD22 binding moiety derived from EPC-001-2 (e.g., scFv) may be any of the anti-CD22 moieties relating to EPC-001-2 disclosed above. In some instances, it comprise the same heavy chain and/or light chain CDRs as EPC-001-2. In specific examples, the scFv may comprise the same V_{H} and/or same V_{L} as EPC-001-2. In some instances, the scFv may comprise, from the N-terminus to the C-terminus, a V_{L} fragment (e.g., SEQ ID NO:17), a peptide linker (e.g., any one of SEQ ID NOs: 38-41), and a V_{H} fragment (e.g., SEQ ID NO:13). Alternatively, the scFv may comprise, from the N-terminus to the C-terminus, a V_{H} fragment (e.g., SEQ ID NO:14), apeptide linker (e.g., any one of SEQ ID NOs: 38-41), and a V_{L} fragment (e.g., SEQ ID NO:17). In one specific example, the anti-CD19 moiety may comprise SEQ ID NO:18.

The anti-CD22 binding moiety derived from EPC-001-3 (e.g., scFv) may be any of the anti-CD22 moieties relating to EPC-001-3 disclosed above. In some instances, it comprise the same heavy chain and/or light chain CDRs as EPC-001-3. In specific examples, the scFv may comprise the same V_{H} and/or same V_{L} as EPC-001-3. In some instances, the scFv may comprise, from the N-terminus to the C-terminus, a V_{L} fragment (e.g., SEQ ID NO:26), a peptide linker (e.g., any one of SEQ ID NOs: 38-41), and a V_{H} fragment (e.g., SEQ ID NO:22). Alternatively, the scFv may comprise, from the N-terminus to the C-terminus, a V_{H} fragment (e.g., SEQ ID NO:22), apeptide linker (e.g., any one of SEQ ID NOs: 38-41), and a V_{L} fragment (e.g., SEQ ID NO:26). In one specific example, the anti-CD19 moiety may comprise SEQ ID NO:27.

The anti-CD22 binding moiety derived from EPC-001-4 (e.g., scFv) may be any of the anti-CD22 moieties relating to EPC-001-3 disclosed above. In some instances, it comprise the same heavy chain and/or light chain CDRs as EPC-001-4. In specific examples, the scFv may comprise the same V_{H} and/or same V_{L} as EPC-001-4. In some instances, the scFv may comprise, from the N-terminus to the C-terminus, a V_{L} fragment (e.g., SEQ ID NO:35), a peptide linker (e.g., any one of SEQ ID NOs: 38-41), and a V_{H} fragment (e.g., SEQ ID NO:31). Alternatively, the scFv may comprise, from the N-terminus to the C-terminus, a V_{H} fragment (e.g., SEQ ID NO:31), apeptide linker (e.g., any one of SEQ ID NOs: 38-41), and a V_{L} fragment (e.g., SEQ ID NO:35). In one specific example, the anti-CD19 moiety may comprise SEQ ID NO:36.

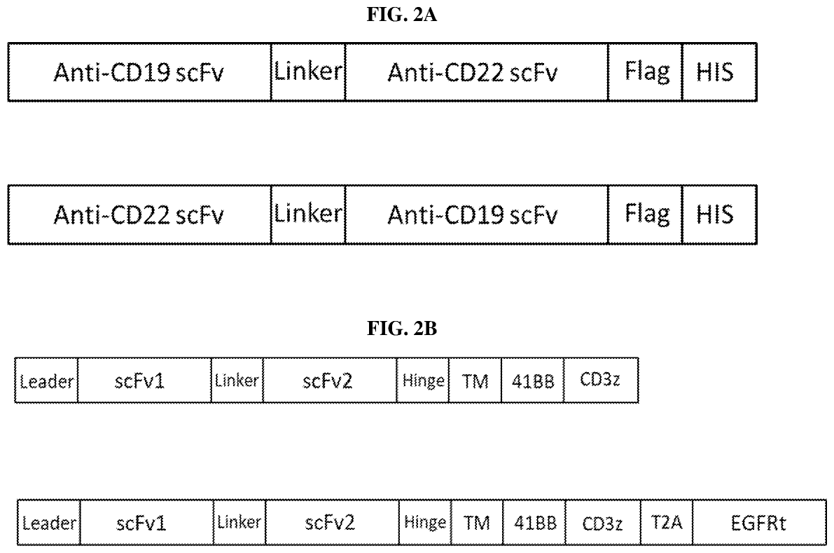

In some embodiments, the anti-CD19/CD22 bispecific CAR may comprise a fusion polypeptide that comprises both the anti-CD19 moiety and the anti-CD22 moiety as disclosed herein, which can be connected via a flexible peptide linker, e.g., any one of SEQ ID NOs: 38-41. In some instances, the linker can be a short G/S rich linker (e.g., having up to 5 amino acid residues), for example, GGGGS (SEQ ID NO: 38). The anti-CD19 and anti-CD22 moieties may be of any orientation as illustrated in FIG. 2A. For example, the fusion polypeptide may comprise, from N-terminus to C-terminus, the anti-CD19 moiety (e.g., anti-CD19 scFv), the peptide linker, and the anti-CD22 moiety (e.g., anti-CD22 scFv). Alternatively, the fusion polypeptide may comprise, from N-terminus to C-terminus, the anti-CD22 moiety (e.g., anti-CD22 scFv), the peptide linker, and the anti-CD19 moiety (e.g., anti-CD19 scFv).

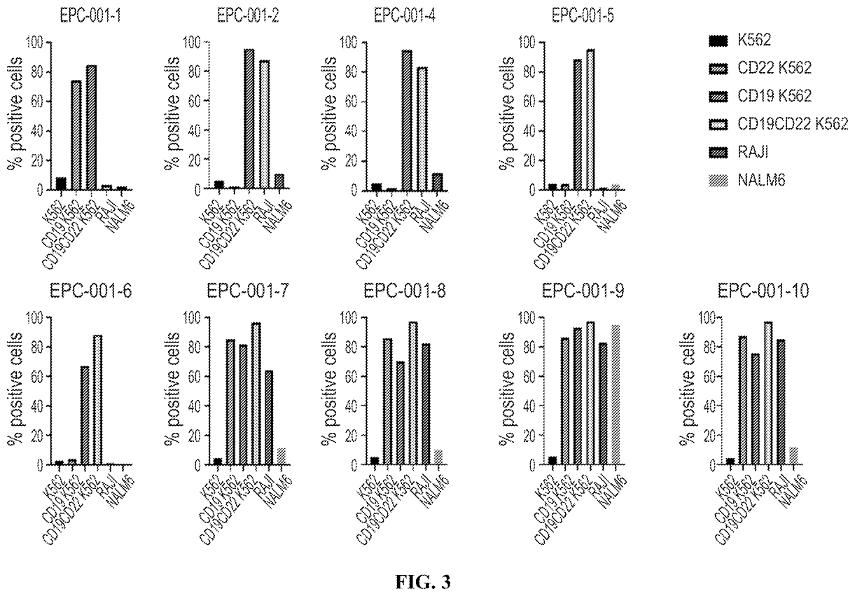

Any of the fusion polypeptide comprising the anti-CD19 and anti-CD22 moieties may further comprise a co-stimulatory signaling domain and a cytoplasmic signaling domain such as those disclosed herein. Optionally, the fusion polypeptide may further comprise a hinge domain and a transmembrane domain as also disclosed herein. A schematic illustration of an exemplary design of a bispecific CAR is provided in FIG. 3. In some examples, the bispecific CAR can be included in a multi-cistronic expression cassette with a suicide gene (e.g., a truncated EGFR) via a self-cleavage peptide linker as illustrated in FIG. 3.

Exemplary anti-CD19/CD22 bispecific scFv and CARs are provided in Table 4 below.

TABLE 4
Exemplary Bispecific scFv and CAR Constructs

| Name of CAR | Amino Acid Sequence | SEQ ID |
|---|---|---|
| EPC-001-5 | QVQLQQWGAGLLKPSETLSLTCAVYGGSFSGYYWTWIRQPPGKGLEWIGEINHGGS | 48 |
| (bispecific | SNYNPSLKSRVTISVDTSKKQFSLNLNSVTAADTAVYYCARGLGYRSGWYEVENAF | |
| scFv) | DIWGQGTMVTVSSGGGGSGGGGSGGGGSQPVLTQPPSVSVAPGQTARITCGGNKIE | |
| | SRSVHWYQQKPGQAPVLVVYDDGARPSGIPERLSGSNSGDTATLTISRVEPGDEAD | |
| | YYCQVWDGSSVIFGGGTKLTVLGGGGSSGGGGSGGGGGGGGSQVQLVQSGAEVKRP | |
| | GASVKVSCKASGYTFTSYGISWVRQAPGQGLEWMGWISAYNGNTNYAQKLQGRVTM | |
| | TTDTSTSTAYMELRSLRSDDTAVYYCARDPGIAVAGTVDYWGQGTLVTVSSGGGGS | |
| | GGGGSGGGGSEIVMTQSPATLSVSPGEGVTLSCRASQSVSSNLAWYQQRPGQAPRL | |
| | LIYGASIKATDVPDRESGGGSGTDFTLSISNVQSEDFAVYYCQQYHTWTPVTFGGG | |
| | TKVEIKDYKDDDDKGGHHHHHH | |
| EPC-001-6 | QVQLVQSGAEVKRPGASVKVSCKASGYTFTSYGISWVRQAPGQGLEWMGWISAYNG | 49 |
| (bispecific | NTNYAQKLQGRVTMTTDTSTSTAYMELRSLRSDDTAVYYCARDPGIAVAGTVDYWG | |
| scFv) | QGTLVTVSSGGGGSGGGGSGGGGSEIVMTQSPATLSVSPGEGVTLSCRASQSVSSN | |
| | LAWYQQRPGQAPRLLIYGASIKATDVPDRFSGGGSGTDFTLSISNVQSEDFAVYYC | |
| | QQYHTWTPVTFGGGTKVEIKGSTSGSGKPGSGEGSTKGQVQLQQWGAGLLKPSETL | |
| | SLTCAVYGGSFSGYYWTWIRQPPGKGLEWIGEINHGGSSNYNPSLKSRVTISVDTS | |
| | KKQFSLNLNSVTAADTAVYYCARGLGYRSGWYEVENAFDIWGQGTMVTVSSGGGGS | |
| | GGGGSGGGGSQPVLTQPPSVSVAPGQTARITCGGNKIESRSVHWYQQKPGQAPVLV | |
| | VYDDGARPSGIPERLSGSNSGDTATLTISRVEPGDEADYYCQVWDGSSVIFGGGTK | |
| | LTVLDYKDDDDKGGHHHHHH | |
| EPC-001-7 | QVQLQQWGAGLLKPSETLSLTCAVYGGSFSGYYWTWIRQPPGKGLEWIGEINHGGS | 50 |
| (bispecific | SNYNPSLKSRVTISVDTSKKQFSLNLNSVTAADTAVYYCARGLGYRSGWYEVENAF | |
| scFv) | DIWGQGTMVTVSSGGGGSGGGGSGGGGSQPVLTQPPSVSVAPGQTARITCGGNKIE | |
| | SRSVHWYQQKPGQAPVLVVYDDGARPSGIPERLSGSNSGDTATLTISRVEPGDEAD | |
| | YYCQVWDGSSVIFGGGTKLTVLGGGGSGGGGSGGGGSGGGGSEVQLVQSGGGVVQP | |
| | GKSLRLSCAASGFTFSSYGMHWVRQAPGKGLEWVAVIWYDGSNKYYADSVKGRFTI | |
| | SRDNSKNTLYLQMNSLRAEDTAVYYCARDGWTGFDYWGQGTTVTVSSGGGGSGGGG | |
| | SGGGGSEIVLTQSPATLSVSPGEGVTLSCRASQSVSSNLAWYQQRPGQAPRLLIYG | |
| | ASIKATDVPDRESGGGSGTDFTLSISNLQSEDFAVYYCQQYHTWPPVTFGGGTKVE | |
| | IKDYKDDDDKGGHHHHHH | |
| EPC-001-8 | EVQLVQSGGGVVQPGKSLRLSCAASGFTFSSYGMHWVRQAPGKGLEWVAVIWYDGS | 51 |
| (bispecific | NKYYADSVKGRFTISRDNSKNTLYLQMNSLRAEDTAVYYCARDGWTGFDYWGQGTT | |
| scFv) | VTVSSGGGGSGGGSGGGGGSEIVLTQSPATLSVSPGEGVTLSCRASQSVSSNLAWY | |
| | QQRPGQAPRLLIYGASIKATDVPDRFSGGGSGTDFTLSISNLQSEDFAVYYCQQYH | |
| | TWPPVTFGGGTKVEIKGSTSGSGKPGSGEGSTKGQVQLQQWGAGLLKPSETLSLTC | |
| | AVYGGSFSGYYWTWIRQPPGKGLEWIGEINHGGSSNYNPSLKSRVTISVDTSKKQF | |
| | SLNLNSVTAADTAVYYCARGLGYRSGWYEVENAFDIWGQGTMVTVSSGGGGSGGGG | |
| | SGGGGSQPVLTQPPSVSVAPGQTARITCGGNKIESRSVHWYQQKPGQAPVLVVYDD | |
| | GARPSGIPERLSGSNSGDTATLTISRVEPGDEADYYCQVWDGSSVIFGGGTKLTVL | |
| | DYKDDDDKGGHHHHHH | |
| EPC-001-9 | QVQLQQWGAGLLKPSETLSLTCAVYGGSFSGYYWTWIRQPPGKGLEWIGEINHGGS | 52 |
| (bispecific | SNYNPSLKSRVTISVDTSKKQFSLNLNSVTAADTAVYYCARGLGYRSGWYEVENAF | |
| scFv) | DIWGQGTMVTVSSGGGGSGGGGSGGGGSQPVLTQPPSVSVAPGQTARITCGGNKIE | |
| | SRSVHWYQQKPGQAPVLVVYDDGARPSGIPERLSGSNSGDTATLTISRVEPGDEAD | |
| | YYCQVWDGSSVIFGGGTKLTVLGGGGSGGGGSGGGGSGGGGSEVQLVQSGAEVKKP | |
| | GASVKVSCKASGYTFTSYGISWVRQAPGQGLEWMGWISAYNGNTNYAQKLQGRVTM | |
| | TTDTSTSTAYMELRSLRSDDTAVYYCARDYGDPSGDDYWGQGTLVTVSSGGGGSGG | |
| | GGSGGGGSEIVLTQSPATLSVSPGEGVTLSCRASQSVSSNLAWYQQRPGQAPRLLI | |
| | YGASIKATDVPDRFSGGGSGTDFTLSISNLQSEDFAVYYCQQYHTWPPVTFGGGTK | |
| | VEIKDYKDDDDKGGHHHHHH | |
| EPC-001-10 | EVQLVQSGAEVKKPGASVKVSCKASGYTFTSYGISWVRQAPGQGLEWMGWISAYNG | 53 |
| (bispecific | NTNYAQKLQGRVTMTTDTSTSTAYMELRSLRSDDTAVYYCARDYGDPSGDDYWGQG | |
| scFv) | TLVTVSSGGGGSGGGGSGGGGSEIVLTQSPATLSVSPGEGVTLSCRASQSVSSNLA | |
| | WYQQRPGQAPRLLIYGASIKATDVPDRFSGGGSGTDFTLSISNLQSEDFAVYYCQQ | |
| | YHTWPPVTFGGGTKVEIKGSTSGSGKPGSGEGSTKGQVQLQQWGAGLLKPSETLSL | |
| | TCAVYGGSFSGYYWTWIRQPPGKGLEWIGEINHGGSSNYNPSLKSRVTISVDTSKK | |
| | QFSLNLNSVTAADTAVYYCARGLGYRSGWYEVENAFDIWGQGTMVTVSSGGGGSGG | |
| | GGSGGGGSQPVLTQPPSVSVAPGQTARITCGGNKIESRSVHWYQQKPGQAPVLVVY | |
| | DDGARPSGIPERLSGSNSGDTATLTISRVEPGDEADYYCQVWDGSSVIFGGGTKLT | |
| | VLDYKDDDDKGGHHHHHH | |
| EPC-001-11 | MLLLVTSLLLCELPHPAFLLIPQVQLQQWGAGLLKPSETLSLTCAVYGGSFSGYYW | 55 |
| (bispecific | TWIRQPPGKGLEWIGEINHGGSSNYDPSLKSRVTISVDTSKKQFSLNLNSVTAADT | |
| CAR; co- | AVYYCARGLGYRSGWYEVENAFDIWGQGTMVTVSSGGGGSGGGGSGGGGSQPVLTQ | |
| expressed with | PPSVSVAPGQTARITCGGNKIESRSVHWYQQKPGQAPVLVVYDDGARPSGIPERLS | |
| EGFR) | GSNSGDTATLTISRVEPGDEADYYCQVWDGSSVIFGGGTKLTVLGSTSGSGKPGSG | |
| | EGSTKGQVQLVQSGAEVKRPGASVKVSCKASGYTFTSYGISWVRQAPGQGLEWMGW | |
| | ISAYNGNTNYAQKLQGRVTMTTDTSTSTAYMELRSLRSDDTAVYYCARDPGIAVAG | |
| | TVDYWGQGTLVTVSSGGGGSGGGGSGGGGSEIVMTQSPATLSVSPGEGVTLSCRAS | |
| | QSVSSNLAWYQQRPGQAPRLLIYGASIKATDVPDRFSGGGSGTDFTLSISNVQSED | |
| | FAVYYCQQYHTWTPVTFGGGTKVEIKESKYGPPCPPCPMFWVLVVVGGVLACYSLL | |
| | VTVAFIIFWVKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| | LEGGGEGRGSLLTCGDVEENPGPRMLLLVTSLLLCELPHPAFLLIPRKVCNGIGIG | 54 |
| | EFKDSLSINATNIKHFKNCTSISGDLHILPVAFRGDSFTHTPPLDPQELDILKTVK | |
| | EITGFLLIQAWPENRTDLHAFENLEIIRGRTKQHGQFSLAVVSLNITSLGLRSLKE | |
| | ISDGDVIISGNKNLCYANTINWKKLFGTSGQKTKIISNRGENSCKATGQVCHALCS | |
| | PEGCWGPEPRDCVSCRNVSRGRECVDKCNLLEGEPREFVENSECIQCHPECLPQAM | |
| | NITCTGRGPDNCIQCAHYIDGPHCVKTCPAGVMGENNTLVWKYADAGHVCHLCHPN | |
| | CTYGCTGPGLEGCPTNGPKIPSIATGMVGALLLLLVVALGIGLFM | |
| EPC-001-12 | MLLLVTSLLLCELPHPAFLLIPQVQLVQSGAEVKRPGASVKVSCKASGYTFTSYGI | 56 |
| (bispecific | SWVRQAPGQGLEWMGWISAYNGNTNYAQKLQGRVTMTTDTSTSTAYMELRSLRSDD | |
| CAR; co- | TAVYYCARDPGIAVAGTVDYWGQGTLVTVSSGGGGSGGGGSGGGGSEIVMTQSPAT | |
| expressed with | LSVSPGEGVTLSCRASQSVSSNLAWYQQRPGQAPRLLIYGASIKATDVPDRFSGGG | |
| EGFR) | SGTDFTLSISNVQSEDFAVYYCQQYHTWTPVTFGGGTKVEIKGSTSGSGKPGSGEG | |
| | STKGQVQLQQWGAGLLKPSETLSLTCAVYGGSFSGYYWTWIRQPPGKGLEWIGEIN | |
| | HGGSSNYDPSLKSRVTISVDTSKKQFSLNLNSVTAADTAVYYCARGLGYRSGWYEV | |
| | ENAFDIWGQGTMVTVSSGGGGSGGGGSGGGGSQPVLTQPPSVSVAPGQTARITCGG | |
| | NKIESRSVHWYQQKPGQAPVLVVYDDGARPSGIPERLSGSNSGDTATLTISRVEPG | |
| | DEADYYCQVWDGSSVIFGGGTKLTVLESKYGPPCPPCPMFWVLVVVGGVLACYSLL | |
| | VTVAFIIFWVKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPPR | |
| | See Above | 54 |
| EPC-001-13 | MLLLVTSLLLCELPHPAFLLIPQVQLQQWGAGLLKPSETLSLTCAVYGGSFSGYYW | 57 |
| (bispecific | TWIRQPPGKGLEWIGEINHGGSSNYDPSLKSRVTISVDTSKKQFSLNLNSVTAADT | |
| CAR; co- | AVYYCARGLGYRSGWYEVENAFDIWGQGTMVTVSSGGGGSGGGGSGGGGSQPVLTQ | |
| expressed with | PPSVSVAPGQTARITCGGNKIESRSVHWYQQKPGQAPVLVVYDDGARPSGIPERLS | |
| EGFR) | GSNSGDTATLTISRVEPGDEADYYCQVWDGSSVIFGGGTKLTVLGSTSGSGKPGSG | |
| | EGSTKGEVQLVQSGGGVVQPGKSLRLSCAASGFTFSSYGMHWVRQAPGKGLEWVAV | |
| | IWYDGSNKYYADSVKGRFTISRDNSKNTLYLQMNSLRAEDTAVYYCARDGWTGFDY | |
| | WGQGTTVTVSSGGGGSGGGGSGGGGSEIVLTQSPATLSVSPGEGVTLSCRASQSVS | |
| | SNLAWYQQRPGQAPRLLIYGASIKATDVPDRFSGGGSGTDFTLSISNLQSEDFAVY | |
| | YCQQYHTWPPVTFGGGTKVEIKESKYGPPCPPCPMFWVLVVVGGVLACYSLLVTVA | |
| | FIIFWVKRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPR | |
| | See Above | 54 |
| EPC-001-14 | MLLLVTSLLLCELPHPAFLLIPEVQLVQSGGGVVQPGKSLRLSCAASGFTFSSYGM | 58 |
| (bispecific | HWVRQAPGKGLEWVAVIWYDGSNKYYADSVKGRFTISRDNSKNTLYLQMNSLRAED | |
| CAR; co- | TAVYYCARDGWTGFDYWGQGTTVTVSSGGGGSGGGGSGGGGSEIVLTQSPATLSVS | |
| expressed with | PGEGVTLSCRASQSVSSNLAWYQQRPGQAPRLLIYGASIKATDVPDRFSGGGSGTD | |
| EGFR) | FTLSISNLQSEDFAVYYCQQYHTWPPVTFGGGTKVEIKGSTSGSGKPGSGEGSTKG | |
| | QVQLQQWGAGLLKPSETLSLTCAVYGGSFSGYYWTWIRQPPGKGLEWIGEINHGGS | |
| | SNYDPSLKSRVTISVDTSKKQFSLNLNSVTAADTAVYYCARGLGYRSGWYEVENAF | |
| | DIWGQGTMVTVSSGGGGSGGGGSGGGGSQPVLTQPPSVSVAPGQTARITCGGNKIE | |
| | SRSVHWYQQKPGQAPVLVVYDDGARPSGIPERLSGSNSGDTATLTISRVEPGDEAD | |
| | YYCQVWDGSSVIFGGGTKLTVLESKYGPPCPPCPMFWVLVVVGGVLACYSLLVTVA | |
| | FIIFWVKRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQEGLYNELQK | |
| | DKMAEAYSEIGMKGERRRGKGHDGLYQGLSTATKDTYDALHMQALPPR | |
| | See Above | 54 |
| EPC-001-15 | MLLLVTSLLLCELPHPAFLLIPQVQLQQWGAGLLKPSETLSLTCAVYGGSFSGYYW | 59 |
| (bispecific | TWIRQPPGKGLEWIGEINHGGSSNYDPSLKSRVTISVDTSKKQFSLNLNSVTAADT | |
| CAR; co- | AVYYCARGLGYRSGWYEVENAFDIWGQGTMVTVSSGGGGSGGGGSGGGGSQPVLTQ | |
| expressed with | PPSVSVAPGQTARITCGGNKIESRSVHWYQQKPGQAPVLVVYDDGARPSGIPERLS | |
| EGFR) | GSNSGDTATLTISRVEPGDEADYYCQVWDGSSVIFGGGTKLTVLGSTSGSGKPGSG | |
| | EGSTKGEVQLVQSGAEVKKPGASVKVSCKASGYTFTSYGISWVRQAPGQGLEWMGW | |
| | ISAYNGNTNYAQKLQGRVTMTTDTSTSTAYMELRSLRSDDTAVYYCARDYGDPSGD | |
| | DYWGQGTLVTVSSGGGGSGGGGSGGGGSEIVLTQSPATLSVSPGEGVTLSCRASQS | |
| | VSSNLAWYQQRPGQAPRLLIYGASIKATDVPDRFSGGGSGTDFTLSISNLQSEDFA | |
| | VYYCQQYHTWPPVTFGGGTKVEIKESKYGPPCPPCPMFWVLVVVGGVLACYSLLVT | |
| | VAFIIFWVKRGRKKLLYIFKQPFMRPVQTTQEEDGCSCRFPEEEEGGCELRVKFSR | |
| | SADAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| | See Above | 54 |
| EPC-001-16 | MLLLVTSLLLCELPHPAFLLIPEVQLVQSGAEVKKPGASVKVSCKASGYTFTSYGI | 60 |
| (bispecific | SWVRQAPGQGLEWMGWISAYNGNTNYAQKLQGRVTMTTDTSTSTAYMELRSLRSDD | |
| CAR; co- | TAVYYCARDYGDPSGDDYWGQGTLVTVSSGGGGSGGGGSGGGGSEIVLTQSPATLS | |
| expressed with | VSPGEGVTLSCRASQSVSSNLAWYQQRPGQAPRLLIYGASIKATDVPDRFSGGGSG | |
| EGFR) | TDFTLSISNLQSEDFAVYYCQQYHTWPPVTFGGGTKVEIKGGGGSGGGGSGGGGSQ | |
| | VQLQQWGAGLLKPSETLSLTCAVYGGSFSGYYWTWIRQPPGKGLEWIGEINHGGSS | |
| | NYNPSLKSRVTISVDTSKKQFSLNLNSVTAADTAVYYCARGLGYRSGWYEVENAFD | |
| | IWGQGTMVTVSSGGGGSGGGGSGGGGSQPVLTQPPSVSVAPGQTARITCGGNKIES | |
| | RSVHWYQQKPGQAPVLVVYDDGARPSGIPERLSGSNSGDTATLTISRVEPGDEADY | |
| | YCQVWDGSSVIFGGGTKLTVLESKYGPPCPPCPMFWVLVVVGGVLACYSLLVTVAF | |
| | IIFWVKRGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| | See Above | 54 |
| EPC-001-17 | MLLLVTSLLLCELPHPAFLLIPEVQLVQSGAEVKKPGASVKVSCKASGYTFTSYGI | 61 |
| (anti-CD22 | SWVRQAPGQGLEWMGWISAYNGNTNYAQKLQGRVTMTTDTSTSTAYMELRSLRSDD | |
| CAR) | TAVYYCARDYGDPSGDDYWGQGTLVTVSSGGGGSGGGGSGGGGSEIVLTQSPATLS | |
| | VSPGEGVTLSCRASQSVSSNLAWYQQRPGQAPRLLIYGASIKATDVPDRFSGGGSG | |
| | TDFTLSISNLQSEDFAVYYCQQYHTWPPVTFGGGTKVEIKESKYGPPCPPCPMFWV | |
| | LVVVGGVLACYSLLVTVAFIIFWVKRGRKKLLYIFKQPFMRPVQTTQEEDGCSCRF | |
| | PEEEEGGCELRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTATKDTYD | |
| | ALHMQALPPRLEGGGEGRGSLLTCGDVEENPGPRMLLLVTSLLLCELPHPAFLLIP | |
| | RKVCNGIGIGEFKDSLSINATNIKHFKNCTSISGDLHILPVAFRGDSFTHTPPLDP | |
| | QELDILKTVKEITGFLLIQAWPENRTDLHAFENLEIIRGRTKQHGQFSLAVVSLNI | |
| | TSLGLRSLKEISDGDVIISGNKNLCYANTINWKKLFGTSGQKTKIISNRGENSCKA | |
| | TGQVCHALCSPEGCWGPEPRDCVSCRNVSRGRECVDKCNLLEGEPREFVENSECIQ | |
| | CHPECLPQAMNITCTGRGPDNCIQCAHYIDGPHCVKTCPAGVMGENNTLVWKYADA | |
| | GHVCHLCHPNCTYGCTGPGLEGCPTNGPKIPSIATGMVGALLLLLVVALGIGLFM | |
| EPC-001-18 | MLLLVTSLLLCELPHPAFLLIPQVQLQQWGAGLLKPSETLSLTCAVYGGSFSGYYW | 62 |
| (anti-CD19 | TWIRQPPGKGLEWIGEINHGGSSNYNPSLKSRVTISVDTSKKQFSLNLNSVTAADT | |
| CAR) | AVYYCARGLGYRSGWYEVENAFDIWGQGTMVTVSSGGGGSGGGGSGGGGSQPVLTQ | |
| | PPSVSVAPGQTARITCGGNKIESRSVHWYQQKPGQAPVLVVYDDGARPSGIPERLS | |
| | GSNSGDTATLTISRVEPGDEADYYCQVWDGSSVIFGGGTKLTVLESKYGPPCPPCP | |
| | MFWVLVVVGGVLACYSLLVTVAFIIFWVKRGRKKLLYIFKQPFMRPVQTTQEEDGC | |
| | SCRFPEEEEGGCELRVKFSRSADAPAYQQGQNQLYNELNLGRREEYDVLDKRRGRD | |
| | PEMGGKPRRKNPQEGLYNELQKDKMAEAYSEIGMKGERRRGKGHDGLYQGLSTATK | |
| | DTYDALHMQALPPRLEGGGEGRGSLLTCGDVEENPGPRMLLLVTSLLLCELPHPAF | |
| | LLIPRKVCNGIGIGEFKDSLSINATNIKHFKNCTSISGDLHILPVAFRGDSFTHTP | |
| | PLDPQELDILKTVKEITGFLLIQAWPENRTDLHAFENLEIIRGRTKQHGQFSLAVV | |
| | SLNITSLGLRSLKEISDGDVIISGNKNLCYANTINWKKLFGTSGQKTKIISNRGEN | |
| | SCKATGQVCHALCSPEGCWGPEPRDCVSCRNVSRGRECVDKCNLLEGEPREFVENS | |
| | ECIQCHPECLPQAMNITCTGRGPDNCIQCAHYIDGPHCVKTCPAGVMGENNTLVWK | |
| | YADAGHVCHLCHPNCTYGCTGPGLEGCPTNGPKIPSIATGMVGALLLLLVVALGIG | |
| | LFM | |
| EPC-001-19 | MLLLVTSLLLCELPHPAFLLIPEVQLVQSGAEVKKPGASVKVSCKASGYTFTSYGI | 63 |
| (bispecific | SWVRQAPGQGLEWMGWISAYNGNTNYAQKLQGRVTMTTDTSTSTAYMELRSLRSDD | |
| CAR; G₄S | TAVYYCARDYGDPSGDDYWGQGTLVTVSSGGGGSGGGGSGGGGSEIVLTQSPATLS | |
| linker; co- | VSPGEGVTLSCRASQSVSSNLAWYQQRPGQAPRLLIYGASIKATDVPDRFSGGGSG | |
| expressed with | TDFTLSISNLQSEDFAVYYCQQYHTWPPVTFGGGTKVEIKGGGGSQVQLQQWGAGL | |
| EGFR) | LKPSETLSLTCAVYGGSFSGYYWTWIRQPPGKGLEWIGEINHGGSSNYNPSLKSRV | |
| | TISVDTSKKQFSLNLNSVTAADTAVYYCARGLGYRSGWYEVENAFDIWGQGTMVTV | |
| | SSGGGGSGGGGSGGGGSQPVLTQPPSVSVAPGQTARITCGGNKIESRSVHWYQQKP | |
| | GQAPVLVVYDDGARPSGIPERLSGSNSGDTATLTISRVEPGDEADYYCQVWDGSSV | |
| | IFGGGTKLTVLESKYGPPCPPCPMFWVLVVVGGVLACYSLLVTVAFIIFWVKRGRK | |
| | KLLYIFKQPFMRPVQTTQEEDGCSCRFPEEEEGGCELRVKFSRSADAPAYQQGQNQ | |
| | LYNELNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| | See Above | 54 |
| EPC-001-20 | MLLLVTSLLLCELPHPAFLLIPEVQLVQSGAEVKKPGASVKVSCKASGYTFTSYGI | 64 |
| (bispecific | SWVRQAPGQGLEWMGWISAYNGNTNYAQKLQGRVTMTTDTSTSTAYMELRSLRSDD | |
| CAR; (G₄S)₂ | TAVYYCARDYGDPSGDDYWGQGTLVTVSSGGGGSGGGGSGGGGSEIVLTQSPATLS | |
| liker; co- | VSPGEGVTLSCRASQSVSSNLAWYQQRPGQAPRLLIYGASIKATDVPDRFSGGGSG | |
| expressed with | TDFTLSISNLQSEDFAVYYCQQYHTWPPVTFGGGTKVEIKGGGGSGGGGSQVQLQQ | |
| EGFR) | WGAGLLKPSETLSLTCAVYGGSFSGYYWTWIRQPPGKGLEWIGEINHGGSSNYNPS | |
| | LKSRVTISVDTSKKQFSLNLNSVTAADTAVYYCARGLGYRSGWYEVENAFDIWGQG | |
| | TMVTVSSGGGGSGGGGSGGGGSQPVLTQPPSVSVAPGQTARITCGGNKIESRSVHW | |
| | YQQKPGQAPVLVVYDDGARPSGIPERLSGSNSGDTATLTISRVEPGDEADYYCQVW | |
| | DGSSVIFGGGTKLTVLESKYGPPCPPCPMFWVLVVVGGVLACYSLLVTVAFIIFWV | |
| | KRGRKKLLYIFKQPFMRPVQTTQEEDGCSCRFPEEEEGGCELRVKFSRSADAPAYQ | |
| | QGQNQLYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| | See Above | 54 |
| EPC-001-21 | MLLLVTSLLLCELPHPAFLLIPEVQLVQSGAEVKKPGASVKVSCKASGYTFTSYGI | 65 |
| (bispecific | SWVRQAPGQGLEWMGWISAYNGNTNYAQKLQGRVTMTTDTSTSTAYMELRSLRSDD | |
| CAR; (G₄S)₃ | TAVYYCARDYGDPSGDDYWGQGTLVTVSSGGGGSGGGGSGGGGSEIVLTQSPATLS | |
| liker; co- | VSPGEGVTLSCRASQSVSSNLAWYQQRPGQAPRLLIYGASIKATDVPDRESGGGSG | |
| expressed with | TDFTLSISNLQSEDFAVYYCQQYHTWPPVTFGGGTKVEIKGGGGSGGGGSGGGGSQ | |
| EGFR) | VQLQQWGAGLLKPSETLSLTCAVYGGSFSGYYWTWIRQPPGKGLEWIGEINHGGSS | |
| | NYNPSLKSRVTISVDTSKKQFSLNLNSVTAADTAVYYCARGLGYRSGWYEVENAFD | |
| | IWGQGTMVTVSSGGGGSGGGGSGGGGSQPVLTQPPSVSVAPGQTARITCGGNKIES | |
| | RSVHWYQQKPGQAPVLVVYDDGARPSGIPERLSGSNSGDTATLTISRVEPGDEADY | |
| | YCQVWDGSSVIFGGGTKLTVLESKYGPPCPPCPMFWVLVVVGGVLACYSLLVTVAF | |
| | IIFWVKRGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| | See Above | 54 |
| EPC-001-22 | MLLLVTSLLLCELPHPAFLLIPEVQLVQSGAEVKKPGASVKVSCKASGYTFTSYGI | 66 |
| (bispecific | SWVRQAPGQGLEWMGWISAYNGNTNYAQKLQGRVTMTTDTSTSTAYMELRSLRSDD | |
| CAR; original | TAVYYCARDYGDPSGDDYWGQGTLVTVSSGGGGSGGGGSGGGGSEIVLTQSPATLS | |
| linker; co- | VSPGEGVTLSCRASQSVSSNLAWYQQRPGQAPRLLIYGASIKATDVPDRESGGGSG | |
| expressed with | TDFTLSISNLQSEDFAVYYCQQYHTWPPVTFGGGTKVEIKGSTSGSGKPGSGEGST | |
| EGFR) | KGQVQLQQWGAGLLKPSETLSLTCAVYGGSFSGYYWTWIRQPPGKGLEWIGEINHG | |
| | GSSNYNPSLKSRVTISVDTSKKQFSLNLNSVTAADTAVYYCARGLGYRSGWYEVEN | |
| | AFDIWGQGTMVTVSSGGGGSGGGGSGGGGSQPVLTQPPSVSVAPGQTARITCGGNK | |
| | IESRSVHWYQQKPGQAPVLVVYDDGARPSGIPERLSGSNSGDTATLTISRVEPGDE | |
| | ADYYCQVWDGSSVIFGGGTKLTVLESKYGPPCPPCPMFWVLVVVGGVLACYSLLVT | |
| | VAFIIFWVKRGRKKLLYIFKQPFMRPVQTTQEEDGCSCRFPEEEEGGCELRVKFSR | |
| | SADAPAYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| | See Above | 54 |
| EPC-001-23 | MLLLVTSLLLCELPHPAFLLIPEVQLVQSGAEVKKPGASVKVSCKASGYTFTSYGI | 63 |
| (bispecific | SWVRQAPGQGLEWMGWISAYNGNTNYAQKLQGRVTMTTDTSTSTAYMELRSLRSDD | |
| CAR; G₄S | TAVYYCARDYGDPSGDDYWGQGTLVTVSSGGGGSGGGGSGGGGSEIVLTQSPATLS | |
| linker) | VSPGEGVTLSCRASQSVSSNLAWYQQRPGQAPRLLIYGASIKATDVPDRFSGGGSG | |
| | TDFTLSISNLQSEDFAVYYCQQYHTWPPVTFGGGTKVEIKGGGGSQVQLQQWGAGL | |
| | LKPSETLSLTCAVYGGSFSGYYWTWIRQPPGKGLEWIGEINHGGSSNYNPSLKSRV | |
| | TISVDTSKKQFSLNLNSVTAADTAVYYCARGLGYRSGWYEVENAFDIWGQGTMVTV | |
| | SSGGGGSGGGGSGGGGSQPVLTQPPSVSVAPGQTARITCGGNKIESRSVHWYQQKP | |
| | GQAPVLVVYDDGARPSGIPERLSGSNSGDTATLTISRVEPGDEADYYCQVWDGSSV | |
| | IFGGGTKLTVLESKYGPPCPPCPMFWVLVVVGGVLACYSLLVTVAFIIFWVKRGRK | |
| | KLLYIFKQPFMRPVQTTQEEDGCSCRFPEEEEGGCELRVKFSRSADAPAYQQGQNQ | |
| | LYNELNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| EPC-001-24 | MLLLVTSLLLCELPHPAFLLIPEVQLVQSGAEVKKPGASVKVSCKASGYTFTSYGI | 67 |
| | SWVRQAPGQGLEWMGWISAYNGNTNYAQKLQGRVTMTTDTSTSTAYMELRSLRSDD | |
| | TAVYYCARDYGDPSGDDYWGQGTLVTVSSGGGGSGGGGSGGGGSEIVLTQSPATLS | |
| | VSPGEGVTLSCRASQSVSSNLAWYQQRPGQAPRLLIYGASIKATDVPDRFSGGGSG | |
| | TDFTLSISNLQSEDFAVYYCQQYHTWPPVTFGGGTKVEIKGGGGSQVQLQQWGAGL | |
| | LKPSETLSLTCAVYGGSFSGYYWTWIRQPPGKGLEWIGEINHGGSSNYDPSLKSRV | |
| | TISVDTSKKQFSLNLNSVTAADTAVYYCARGLGYRSGWYEVENAFDIWGQGTMVTV | |
| | SSGGGGSGGGGSGGGGSQPVLTQPPSVSVAPGQTARITCGGNKIESRSVHWYQQKP | |
| | GQAPVLVVYDDGARPSGIPERLSGSNSGDTATLTISRVEPGDEADYYCQVWDGSSV | |
| | IFGGGTKLTVLESKYGPPCPPCPMFWVLVVVGGVLACYSLLVTVAFIIFWVKRGRK | |
| | KLLYIFKQPFMRPVQTTQEEDGCSCRFPEEEEGGCELRVKFSRSADAPAYQQGQNQ | |
| | LYNELNLGRREEYDVLDKRRGRDPEMGGKPRRKNPQEGLYNELQKDKMAEAYSEIG | |
| | MKGERRRGKGHDGLYQGLSTATKDTYDALHMQALPPR | |
| EGFRt | RKVCNGIGIGEFKDSLSINATNIKHFKNCTSISGDLHILPVAFRGDSFTHTPPLDP | 68 |
| | QELDILKTVKEITGFLLIQAWPENRTDLHAFENLEIIRGRTKQHGQFSLAVVSLNI | |
| | TSLGLRSLKEISDGDVIISGNKNLCYANTINWKKLFGTSGQKTKIISNRGENSCKA | |
| | TGQVCHALCSPEGCWGPEPRDCVSCRNVSRGRECVDKCNLLEGEPREFVENSECIQ | |
| | CHPECLPQAMNITCTGRGPDNCIQCAHYIDGPHCVKTCPAGVMGENNTLVWKYADA | |
| | GHVCHLCHPNCTYGCTGPGLEGCPTNGPKIPSIATGMVGALLLLLVVALGIGLFM | |

Also within the scope of the present disclosure are bi-specific anti-CD19/CD22 antibodies comprising an anti-CD19 binding moiety derived from the parent anti-CD19 antibody provided in Table 1 and an anti-CD22 binding moiety derived from the parent anti-CD22 antibody provided in Table 2 herein. Such bi-specific antibodies may be in any suitable format as known in the art. For example, the bi-specific antibodies may comprise an anti-CD19 scFv and an anti-CD22 scFv in tandem repeat (e.g., the bi-specific antigen binding moiety in any of the bi-specific CARs disclosed herein). In some instances, such a bi-specific antibody may further comprise an Fc fragment to form an scFv-Fc fusion polypeptide.

### (e) Anti-CD19 and Anti-CD22 CARs

Also within the scope are anti-CD19 and anti-CD22 CARs comprising any of the anti-CD19 binding moieties and anti-CD22 binding moieties as disclosed herein.

In some aspects, provided herein are anti-CD19 CAR, nucleic acids encoding such, and host cells expressing such. The anti-CD19 CAR may comprise (a) an extracellular binding domain which can be any of the anti-CD19 binding moieties, e.g., an anti-CD19 scFv derived from EPC-001-1; (b) a co-stimulatory signaling domain such as those disclosed herein; and (c) a cytoplasmic signaling domain such as those disclosed herein. The anti-CD19 CAR may further comprise a hinge domain and a transmembrane domain located at the C-terminal of the extracellular antigen binding domain. In one example, the anti-CD19 CAR comprises the amino acid sequence of SEQ ID NO: 62.

In some aspects, provided herein are anti-CD22 CAR, nucleic acids encoding such, and host cells expressing such. In some examples, the anti-CD22 CAR may comprise (a) an extracellular binding domain which can be any of the anti-CD22 binding moieties, e.g., an anti-CD19 scFv derived from EPC-001-2, EPC-001-3, or EPC-001-4; (b) a co-stimulatory signaling domain such as those disclosed herein; and (c) a cytoplasmic signaling domain such as those disclosed herein. The anti-CD22 CAR may further comprise a hinge domain and a transmembrane domain located at the C-terminal of the extracellular antigen binding domain In one example, the anti-CD22 CAR comprises the amino acid sequence of SEQ ID NO: 61.

### II. CAR-Expressing Immune Cells

In some aspects, provided herein are genetically engineered immune cells such as T cells NK cells, or macrophages having surface expression of any of the anti-CD19, anti-CD22, or anti-CD19/CD22 bispecific CAR constructs disclosed herein. In some instances, the genetically engineered immune cells are T cells expressing any of the anti-CD19/CD22 bispecific CAR provided in Table 4 above (e.g., SEQ ID NO:63).

Any of the CAR-expression immune cells disclosed herein may be engineered with additional mechanisms to reprogram the CAR-expressing cells so as to enhance their bioactivity and/or persistence, thereby enhancing overall therapeutic effects. For example, the CAR-expressing immune cells may be further engineered to knock-in one or more immunomodulator genes, one or more immune checkpoint inhibitor genes, or a combination thereof. Alternatively, the CAR-expressing immune cells disclosed herein may be further engineered to knock down or knock out one or more inhibitory genes.

### (a) Preparation of CAR-Expressing Immune Cells

The genetically engineered immune cells disclosed herein may be prepared by introducing an expression cassette encoding any of the CAR constructs disclosed herein (e.g., any of the anti-CD19/CD22 bispecific CARs disclosed herein such as those provided in Table 4) into suitable immune cells and collecting the resultant engineered immune cells that express the CAR on cell surface.

A population of immune cells, as the starting parent cells, can be obtained from any source, such as peripheral blood mononuclear cells (PBMCs), bone marrow, or tissues such as spleen, lymph node, thymus, stem cells, or tumor tissue. A source suitable for obtaining the type of host cells desired would be evident to one of skill in the art. In some embodiments, the population of immune cells is derived from PBMCs. The type of host cells desired (e.g., T cells, NK cells, macrophages, or a combination thereof) may be expanded within the population of cells obtained by co-incubating the cells with stimulatory molecules. As a non-limiting example, anti-CD3 and anti-CD28 antibodies may be used for expansion of T cells. In some embodiments, a specific type of cells (e.g., T cells, NK cells, or macrophages) may be enriched from the immune cell population. Such enriched cell subpopulation may be expanded and/or activated in vitro prior to the genetic engineered for introduction of the CAR-encoding expression cassette.

To construct the immune cells that express any of the CAR polypeptides described herein (e.g., any of the anti-CD19/CD22 bispecific CARs disclosed herein such as those provided in Table 4), expression vectors for stable or transient expression of the CAR polypeptide may be created via conventional methods and introduced into immune host cells. For example, nucleic acids encoding the CAR polypeptides may be cloned into a suitable expression vector, such as a viral vector in operable linkage to a suitable promoter. Non-limiting examples of useful vectors of the disclosure include viral vectors such as, e.g., retroviral vectors including gamma retroviral vectors, adeno-associated virus vectors (AAV vectors), and lentiviral vectors. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the CAR polypeptides. The synthetic linkers may contain nucleic acid sequences that correspond to a particular restriction site in the vector. The selection of expression vectors/plasmids/viral vectors would depend on the type of host cells for expression of the CAR polypeptides but should be suitable for integration and replication in eukaryotic cells. Any of such nucleic acids encoding the CAR and expression vectors comprising such are also within the scope of the present disclosure.

A variety of promoters can be used for expression of the CAR polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, or herpes simplex tk virus promoter. Additional promoters for expression of the CAR polypeptides include any constitutively active promoter in an immune cell. Alternatively, any regulatable promoter may be used, such that its expression can be modulated within an immune cell. In some embodiments, the promoter can be the pEFlu promoter.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the CAR polypeptide.

In one specific embodiment, such vectors may also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, nitroreductase, and caspases such as caspase 8. In specific examples, the suicide gene may encode a truncated EGFR, for example, the truncated EGFR provided in Table 3 and Table 4 above.

The nucleic acid disclosed herein may comprise two coding sequences, one for any of the CAR constructs disclosed herein (e.g., any of the anti-CD19/CD22 bispecific CARs disclosed herein such as those provided in Table 4) and the other for the suicide gene product. The two coding sequences may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a self-cleavage peptide such as P2A, T2A, or F2A peptide (see, for example, Kim et al., PLoS One. 2011;6(4):e18556). See also FIG. 3.

Any of the vectors comprising a nucleic acid sequence that encodes an ACTR polypeptide described herein is also within the scope of the present disclosure.

Such a vector, or the sequence encoding a CAR polypeptide contained therein, may be delivered into host cells such as host immune cells (e.g., T cells, NK cells, or macrophages) by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

Following introduction into the host cells a vector encoding any of the CAR polypeptides provided herein (e.g., any of the anti-CD19/CD22 bispecific CARs disclosed herein such as those provided in Table 4), the cells may be cultured under conditions that allow for expression of the CAR polypeptide. When expression of the CAR polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the CAR polypeptide is expressed will be evident to one of skill in the art and may be assessed by any known method, for example, detection of the CAR polypeptide-encoding mRNA by quantitative reverse transcriptase PCR (qRT-PCR) or detection of the CAR polypeptide protein by methods including Western blotting, fluorescence microscopy, and flow cytometry. Alternatively, expression of functional CAR may be determined by binding activity and/or CTL activity against cells expressing the target antigen, e.g., CD19 and/or CD22.

Methods for preparing host cells expressing any of the CAR polypeptides described herein may also comprise activating the host cells ex vivo. Activating a host cell means stimulating a host cell into an activated state in which the cell may be able to perform effector functions. Methods of activating a host cell will depend on the type of host cell used for expression of the CAR polypeptides. For example, T cells may be activated ex vivo in the presence of one or more molecules including, but not limited to: an anti-CD3 antibody, an anti-CD28 antibody, IL-2, and/or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and/or K562 cells. In some embodiments, the host cells expressing any of the CAR polypeptides (CAR-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the CAR polypeptides described herein may comprise expanding the host cells ex vivo. Expanding host cells may involve any method that results in an increase in the number of cells expressing CAR polypeptides, for example, allowing the host cells to proliferate or stimulating the host cells to proliferate. Methods for stimulating expansion of host cells will depend on the type of host cell used for expression of the CAR polypeptides and will be evident to one of skill in the art. In some embodiments, the host cells expressing any of the CAR polypeptides described herein are expanded ex vivo prior to administration to a subject.

In some embodiments, the host cells expressing the CAR polypeptides are expanded and activated ex vivo prior to administration of the cells to the subject. Host cell activation and expansion may be used to allow integration of a viral vector into the genome and expression of the gene encoding a CAR polypeptide as described herein. If mRNA electroporation is used, no activation and/or expansion may be required, although electroporation may be more effective when performed on activated cells.

In some instances, a CAR polypeptide is transiently expressed in a suitable host cell (e.g., for 3-5 days). Transient expression may be advantageous if there is a potential toxicity and should be helpful in initial phases of clinical testing for possible side effects.

### (b) Pharmaceutical Compositions

Any of the genetically engineered immune cells expressing a CAR as disclosed herein (e.g., any of the anti-CD19/CD22 bispecific CARs such as those provided in Table 4 above) may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20 th Ed. (2000) Lippincott Williams and Wilkins, Ed. K. E. Hoover.

For examples of additional useful agents, see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### IV. Therapeutic Applications

Any of the genetically engineered immune cells (e.g., T cells, NK cells, or macrophages) expressing a CAR as disclosed herein (e.g., any of the anti-CD19/CD22 bispecific CARs such as those provided in Table 4 above) may be used for therapeutic purposes, for example, to eliminate undesired cells expressing CD19 and/or CD22. In some examples, the genetically engineered immune cells are CAR-T cells expressing any of the anti-CD19/CD22 bispecific CARs such as those provided in Table 4 above.

To practice the method described herein, an effective amount of the immune cells (NK cells, T lymphocytes, or macrophages) expressing any of the CAR described herein (e.g., any of the anti-CD19/CD22 bispecific CARs such as those provided in Table 4 above), or pharmaceutical compositions thereof may be administered to a subject in need of the treatment via a suitable route, such as intravenous administration. As used herein, an effective amount refers to the amount of the respective agent (e.g., the NK cells, T lymphocytes or macrophages expressing the CAR) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human In some embodiments, the subject in need of treatment is a human cancer patient.

As used herein, the term “therapeutically effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

In some embodiments, the methods of the disclosure may be used for eliminating or inhibiting disease cells expressing CD19 and/or CD22. Accordingly, any of the immune cells disclosed herein may be used for treating a disease associated with CD19⁺ and/or CD22⁺ disease cells, such as CD19⁺ and/or CD22⁺ cancer cells. The method disclosed herein may be used for treating a cancer involving CD19⁺ and/or CD22⁺ cancer cells, for example, a hematopoietic cancer. In certain embodiments, the cancer may be a solid tumor.

In some embodiments, an effective amount of any of the genetically engineered immune cells express a CAR as disclosed herein (e.g., any of the anti-CD19/CD22 bispecific CAR such as those provided in Table 4 above) may be given to a subject in need of the treatment via a suitable route, for example, intravenous infusion. The subject may be a human patient having a disease associated with CD19⁺ and/or CD22⁺ disease cells, such as CD19+ and/or CD22⁺ cancer cells. In some instances, the human patient has a cancer involving CD19⁺ and/or CD22⁺ cancer cells. In some instances, the human patient may have a hematopoietic cancer. In other instances, the human patient may have a solid tumor.

In some examples, the human patient may have a B-cell malignancy, which involves CD19⁺ and/or CD22⁺ disease B cells. Examples include, but are not limited to, non-Hodgkin lymphoma, diffuse large B-cell lymphoma (DLBCL), follicular lymphoma, acute lymphocytic leukemia (ALL), chronic lymphocytic leukemia (CLL), small lymphocytic lymphoma (SLL), mantle cell lymphoma (MCL), marginal zone lymphoma, Burkitt lymphoma, lymphoplasmacytic lymphoma, hairy cell leukemia (HCL), primary central nervous system (CNS) lymphoma, and primary intraocular lymphoma.

In some examples, the human patient may have a T-cell malignancy. Examples include, but are not limited to, T-lymphoblastic lymphoma/leukemia, peripheral T-cell lymphoma (e.g., cutaneous T-cell lymphoma, adult T-cell leukemia, angioimmunoblastic T-cell lymphoma, extranodal natural killer/T-cell lymphoma, enteropathy-associated intestinal T-cell lymphoma (EATL), anaplastic large cell lymphoma (ALCL), or peripheral T-cell lymphoma, not otherwise specified (PTCL, NOS)).

In some embodiments, the immune cells (e.g., NK and/or T cells) for use in the treatment disclosed herein may be autologous to the subject, i.e., the immune cells may be obtained from the subject in need of the treatment, genetically engineered for expression of the CAR polypeptides, and then administered to the same subject. In one specific embodiment, prior to re-introduction into the subject, the autologous immune cells (e.g., T lymphocytes, NK cells, or macrophages) are activated and/or expanded ex vivo. Administration of autologous cells to a subject may result in reduced rejection of the host cells as compared to administration of non-autologous cells.

Alternatively, the genetically engineered immune cells (e.g., T cells, NK cells, or macrophages) can be allogeneic cells, i.e., the cells are obtained from a first subject, genetically engineered for expression of the CAR polypeptide, and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes, in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, and/or phytohemoagglutinin.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line. See, e.g., U.S. Patents Nos. 7,435,596 and 8,026,097 for the description of useful methods for expanding NK cells. For example, NK cells used in the methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)], and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-receptor antibody. Preferably, the support will have IL-15 receptor antibody and CD137 antibody bound on its surface. ⁺ In accordance with the present disclosure, patients can be treated by infusing therapeutically effective doses of immune cells such as T lymphocytes or NK cells expressing a CAR polypeptide such as an anti-CD19/CD22 bispecific CAR as listed in Table 4 above (e.g., SEQ ID NO: 23) in the range of about 10⁵ to 10⁹ CAR+ cells to a patient. The infusion can be repeated as often and as many times as the patient can tolerate until the desired response is achieved. The appropriate infusion dose and schedule will vary from patient to patient but can be determined by the treating physician for a particular patient. In some examples, initial doses of approximately 10⁶ cells/Kg can be infused, escalating to 10⁸ or more cells/Kg.

The particular dosage regimen, i.e., dose, timing and repetition, used in the method described herein will depend on the particular subject and that subject's medical history. The appropriate dosage of the CAR-expressing immune celles used will depend on the type of cancer to be treated, the severity and course of the disease, previous therapy, the patient's clinical history and response to the immune cell therapy, and the discretion of the attending physician.

In some embodiments, the genetically engineered immune cells expressing any of the CAR constructs disclosed herein (e.g., the anti-CD19 CAR, the anti-CD22 CAR, or the anti-CD19/CD22 bispecific CAR) may be utilized in conjunction with other types of therapy for cancer, such as chemotherapy, surgery, radiation, gene therapy, and so forth. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

### V. Kits for Therapeutic Applications

The present disclosure also provides kits for use of the genetically engineered immune cells (e.g., T lymphocytes, NK cells, or macrophages) expressing anti-CD19 CAR, anti-CD22 CAR, or anti-CD19/CD22 bispecific CAR described herein. Such kits may include one or more containers comprising the genetically engineered immune cells, which may be formulated in a pharmaceutical composition further comprising a pharmaceutically acceptable carrier. In some embodiments, the kit described herein comprises genetically engineered immune cells, which may be expanded in vitro. The immune cells may express any of the CAR disclosed herein, for example, any of the anti-CD19/CD22 bispecific CARs such as those provided in Table 4 above.

In some embodiments, the kit can additionally comprise instructions for use in any of the methods described herein. The included instructions may comprise a description of administration of the genetically engineered immune cells disclosed herein to achieve the intended activity, e.g., eliminating the target disease cells such as cancer cells expressing CD19, CD22, or both, in a subject. The kit may further comprise a description of selecting a subject suitable for treatment based on identifying whether the subject is in need of the treatment.

The instructions relating to the use of the genetically engineered immune cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the genetically engineered immune cells are used for treating, delaying the onset, and/or alleviating a disease or disorder associated with CD19 and/or CD22 positive disease cells in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1

### Generation of CD19⁺ and/or CD22⁺ Cells Lines and Characterization Thereof

This example describes generation of cell lines expression one or both of CD19 and CD22 surface antigens

### (a) Generation of CD19/CD22 Positive Recombinant Cell Lines

K562 cells (ATCC) were transfected with 10 ug of pCMV6-Entry vector carrying a nucleotide sequence encoding the full-length human CD19 or CD22 fused with flag or Myc tags at the C-terminus. G418 drug selection process yielded a polyclonal, drug resistant pools of CD19 or CD22 expressing cells. In parallel, the parental cell line transfected with the empty pCMV6-Entry vector was generated for use as a negative control. The CD19 or CD22 expressing cells were sorted by FACS to yield a pool of CD19 or CD22 expressing cells. The pools were expanded under G418 drug selection. Single cell sorting was then performed followed by further drug selection to generate clonal cell lines. The clonal lines were screened for CD19 or CD22 expression by FACS.

To generate CD19/CD22 double positive cell line, 10 ug of CD22 plasmid was transfected into 5M of CD19 expressing cell line and selected under G418. Single cell sorting for both CD19 and CD22 high, G418 selection and clonal FACS screening was performed to obtain high CD19/CD22/K562 double positive cell line.

### (b) Quantification of Recombinant and Endogenous CD19 and CD22 Receptor Numbers in Target Cell Lines

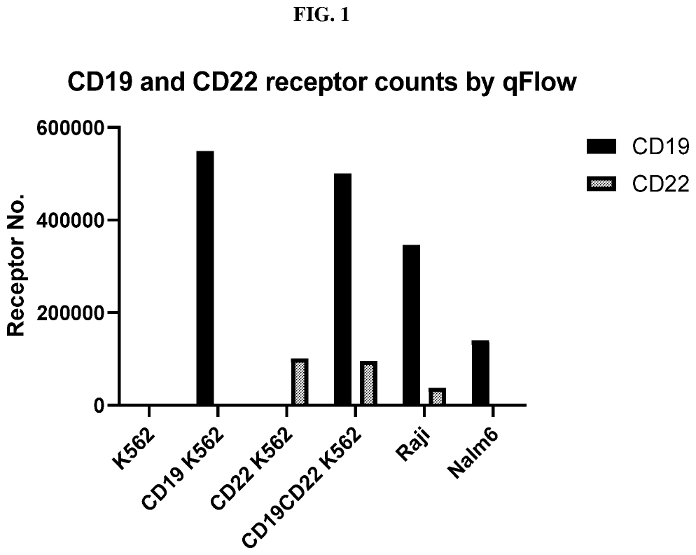

To further characterize the recombinant and endogenous CD19 and CD22 expression levels in target cell lines (as indicated herein), quantification FACS assay was performed using Bangs Laboratories Inc QUANTUM Alexa Fluor® 647 (fluorescent dey) MESF microsphere beads for standard calibration following manufacture's protocol. The parental K562 cell line showed non-detectable CD19 and CD22 expression. The expression level of CD19 was approximately 5-fold higher than that of CD22 in single or double positive recombinant cell lines. Raji cells showed more CD19 and CD22 expression than Nalm 6 cells. In both Raji cells and Nalm 6 cells, the expression levels of CD19 were about 4-9-fold higher that the expression levels of CD22. The CD19 and CD22 receptor copy numbers are summarized in Table 5. See also FIG. 1.

TABLE 5
Copy Numbers of CD19 and CD22 in Target Cell Lines

| | CD19 Receptor No. | CD22 Receptor No. |
|---|---|---|
| K562 | 0 | 0 |
| CD19/K562 | 549231 | 0 |
| CD22/K562 | 0 | 101030 |
| CD19/CD22/K562 | 500923 | 95893 |
| Raji | 346462 | 37887 |
| Nalm6 | 140154 | 3207 |

### (c) Generation of CD19, CD22, and CD19/CD22 GFP Positive Cell Lines

K562, CD19 K562, CD22 K562, CD19/CD22 K562, Raji and Nalm 6 cell lines have been further engineered to introduce a GFP expression cassette using Incucyte CytoLight green lentivirus transduction. Cells were sorted for GFP positive and under G418 drug selection to establish stable cell lines. The GFP positive cell lines were utilized for imaging-based cytotoxicity assays on Cytation® 5 instrument (a cell imaging multimode reader).

### Example 2

### CD19-CD22 Bispecific scFv Characterization

Exemplary anti-CD19/CD22 bispecific antibodies were characterized as follows.

(a) CD19-CD22 Bispecific scFv Preparation

CD19/CD22 bispecific scFvs have been cloned into pET22b bacterial periplasmic vectors in CD19-CD22 and CD22-CD19 orientations and expressed in Rosetta II strain. FIG. 2A illustrates exemplary designs of the CD19/CD22 bispecific scFvs. To purify the antibody fragment expressed in E. coli cells, 3 μl Ni Sepharose® Excel resin (GE) were mixed with 1 mL of filtered supernatant and loaded onto 10 mL or 20 mL BioRad Econo-Pac® columns. Before loading, the resin of the column was equilibrated with at least 20 column volume (CV) buffer A (1×PBS, pH7.4 with extra NaCl added to 500 mM). The filter sterilized supernatant was purified by gravity flow via either controlling the flow to 1 mL/min or being poured over two times, over the same packed resin bed. The column was then washed with the following buffers: 10 CV buffer A, 20 CV buffer B (1×PBS, pH7.4 with extra NaCl to 500 mM, and 30 mM imidazole). Detox® buffers were used to remove endotoxin, if needed.

To purify the antibody fragment from the 250mL expression culture, antibody-bound column was washed sequentially with 20 CV buffer C (1×PBS pH7.4 with extra NaCl to 500 mM, 1% Tx114), 20 CV buffer D (1×PBS pH7.4 with extra NaCl to 500 mM, 1% Tx100 +0.2% TNBP) and 40 CV buffer E (1×PBS pH7.4 with extra NaCl to 500 mM).

The protein was eluted with Eluting buffer F (1×PBS pH7.4 with extra NaCl to 500 mM, and 500 mM imidazole) in a total of six fractions (0.5 CV pre elute, 5× 1 CV elute). Fractions were run on a Bradford assay (100 ul diluted Bradford solution+10 ul sample). Fractions with bright blue color were pooled and the protein concentration thereof was measured by A280 extension coefficient. SDS-PAGE gel assay was performed to analyze the purity of the purified antibodies.

(b) Binding Activity of Anti-CD19, Anti-CD22, and Anti- CD19/CD22 Bispecific scFv Antibodies to Cell Surface Antigens by FACS Analysis

To determine the specific target cell binding activity after converting into different bispecific scFv formats, CD19, CD22 monospecific scFv and CD19/CD22 bispecific scFvs have been tested in FACS binding assays with K562, CD19 K562, CD22 K562 and CD19/CD22 K562 recombinant cell lines. Briefly, each bispecific scFv was diluted to 200 nM and incubated with 100,000 K562, CD19 K562, CD22 K562 and CD19/CD22 K562 cell lines in 96 wells plate at 4° C. for 1 hour with shaking. Cells were spun down at 1300 rpm for 5 minutes at 4° C. to remove unbound antibodies. Cells were then washed once with 200 iaL of PBS per well. Samples were mixed with an Alexa Fluor® 647 (fluorescent dye)-conjugated anti-His antibody (secondary antibody, 100 μL, 1:1000 diluted) and incubated at 4° C. for 30 minutes in dark with shaking. Samples were then spun down at 1300 rpm for 5 minutes at 4° C. and washed twice with 200uL of 1×PBS per well. The resultant samples were reconstituted in 200 uL of 1×PBS and read on Attune™ NxT Flow Cytometer. Analysis was done by counting only Alexa Fluor® 647-(fluorescent dye) positive cells and then plotted in GraphPad Prism® 8.1 software.

Binding activities of the Anti-CD19, Anti-CD22, and Anti- CD19/CD22 Bispecific scFv to cell surface antigens are provided in FIG. 3. Among the bispecific scFvs tested, four bispecific scFvs retained similar binding activity to both CD19 and CD22 target cell lines as parental mono-specific scFvs. Two bispecific scFvs showed similar binding on CD19 target cell line and reduced binding to CD22 target cell line compared to parental monospecific scFvs.

The four bispecific scFvs that have full binding activity were then tested for binding activity to Raji cells, which express endogenous CD19 and CD22 by FACS assay; their ECso values were determined. Briefly, each purified bispecific scFv protein was titrated from 200 nM with 3-fold serial dilutions in full medium. The diluted samples were incubated with 100,000 Raji cell line in 96 wells plate at 4° C. for 1 hour with shaking. The wash, detection and analysis was done as described above. EC50 values of these exemplary anti-CD19/CD22 bispecific scFv antibodies are provided in Table 6 below:

TABLE 6
EC50 Values of Exemplary Bispecific Antibodies

| EC50 nM | EPC-001-7 | EPC-001-8 | EPC-001-9 | EPC-001-10 |
|---|---|---|---|---|
| Raji | 1.923 | 1.713 | 2.235 | 2.522 |

### Example 3

### Construction of Anti-CD19/CD22 Bispecific Chimeric Antigen Receptors

This example describes construction of exemplary anti-CD19/CD22 bispecific chimeric antigen receptors (CARs) and introduction of such constructs to host cells for expression via viral transduction.

### (a) Exemplary CAR Format and Construction

Bispecific CD19/CD22 scFv were converted into CD19-CD22 or CD22-CD19 orientations. The scFv fragments were linked in tandem format and with a modified version of 20 IgG4 hinge, CD28 transmembrane domain, 4-1BB co-stimulatory domain, and CD3z intracellular signaling domain (see Sequence Table below). In some examples, the bispecific CAR construct was further linked to a truncated EGFR fragment. Exemplary bispecific CAR constructs are illustrated in FIG. 2B. The sequences were cloned into pEF1a based lentivial vectors. Table 7 below provides exemplary CAR constructs.

TABLE 7
Exemplary CAR Constructs

| | Orientation | Linker | EGFRt |
|---|---|---|---|
| CAR No. | (N terminus to C | length | Inclusion |
| EPC-001-11 | CD19-CD22 | 18 | Yes |
| EPC-001-12 | CD22-CD19 | 18 | Yes |
| EPC-001-13 | CD19-CD22 | 18 | Yes |
| EPC-001-14 | CD22-CD19 | 18 | Yes |
| EPC-001-15 | CD19-CD22 | 18 | Yes |
| EPC-001-16 | CD22-CD19 | 18 | Yes |

### (b) Lentivirus Production and Characterization

Bispecific CAR-encoding lentiviral vectors were co-transfected with LV-MAX packaging mix using polyethylenimine (PEI) transfection reagents to Expi293™ (HEK293 cells) following manufacture's protocol. Transfected cells were grown for 72 hrs at 37° C. shaking with 8% CO2 level. Supernatant were harvested by centrifugation at 3200 rpm at RT for 10 mins and vacuum filtration using 0.45um PES membrane. Virus were concentrated by ultracentrifugation (Beckman Coulter) at 18000 rpm for 2 hrs at 4° C. The pellet was then resuspended in Lentivirus stabilizer, aliquoted immediately and stored at −80° C.

Virus titers were measured using p24 ELISA kit (Qiagen) following manufacture's protocol and calculated based on the standard curve set up in each assay. Functional titer TU/mL was measured by transducing different virus amount to fixed number of HEK293 cells based on P24 ELISA results. Percentage of CAR+ expression cells were checked by flow cytometry post transfection at different timepoints starting from 24 hours.

### Example 4

### Characterization of Immune Cells Expressing Anti-CD19/CD22 Bispecific CAR

PBMCs were isolated from fresh healthy donor's in a LRS chamber using density gradient centrifugation Lymphoprep™ (a density gradient medium) and SepMate™ 50 PBMC isolation kit from Stemcell Technology. CD3⁺ Pan T cells were then isolated from PBMCs using EasySep™ (a density gradient medium) human T cell isolation kit following Stemcell technology protocols. Pan T cells were activated with human T-activator CD3/CD28 Dynabeads® beads at 1:1 bead to cell ratio 24 hours and then transduced with lentivirus in the presence of Dynabeads (beads conjugated with anti-CD3/CD28 antibodies) and 1 mg/mL protamine sulfate. Spinoculation was done at 300 g for 2 hours at 25° C. Cells and viruses were incubated for 24 hours at 37° C. Next day, cells were removed from beads and viruses. Cells were grown for 2 weeks in 5% human serum containing recombinant human IL15 and IL7 (Peprotech) in X-VIVO™ 15 (Lonza) media. Media were changed every 2-3 days with added fresh cytokines.

Expression of CAR on cell surface was assessed by surface staining using an anti-EGFR antibody or CD22-Fc directly conjugated with Mix-n-stain Alexa Fluor® 647 or CF 640R antibody labeling kit (Sigma). Briefly, 100,000 lentivirus transduced T cells were incubated with 25 nM of anti-EGFR-Alexa Fluor® 647 or recombinant human CD22/Fc-CF 640R for 1 hour in dark at 4° C. shaking. Cells were spined down at 1,300 rpm for 5 minutes, supernatant removed and washed with 200 uL 1×PBS. The resultant samples were reconstituted in 200 uL of 1×PBS. The percentage surface expression was quantified by reading the fluorescence stained cells on Attune™ NxT Flow Cytometer. CAR surface expression was also imaged on Cytation® 5 instrument at 20× magnification with Cy5 cube and DAPI cube (BioTek). 1:1000 dilution Hoechst 34580 was used to stain nucleus of the cells.

CAR-expression level of different CAR constructs on human T cells ranges from 35-85% detected by conjugated anti-EGFR or CD22-Fc recombinant protein in FACS assays. Table 8 below summarizes percentage of CAR+ cells in PMBCs transduced with the listed CAR construct.

TABLE 8
Percentages of CAR+ Cells

| CAR Constructs | Positive Cell % |
|---|---|
| EPC-001-11 | 54.577 |
| EPC-001-12 | 36.180 |
| EPC-001-13 | 62.423 |
| EPC-001-14 | 66.784 |
| EPC-001-15 | 67.659 |
| EPC-001-16 | 84.042 |

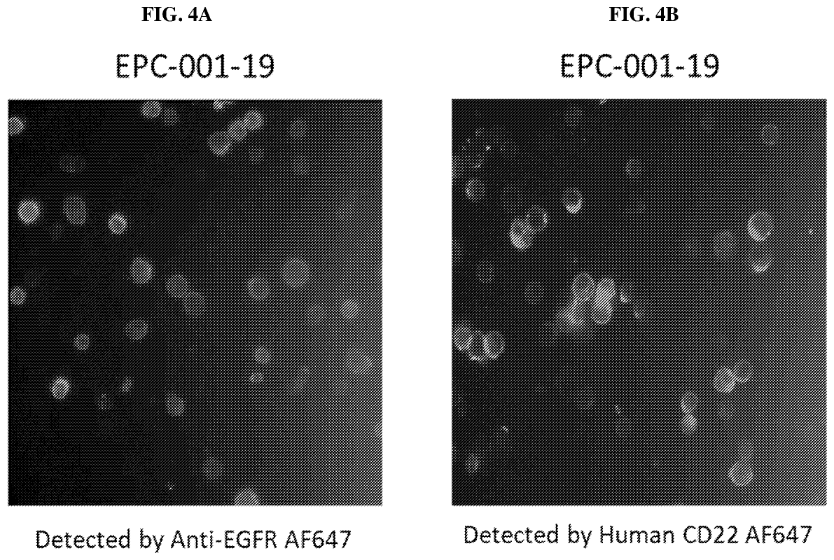

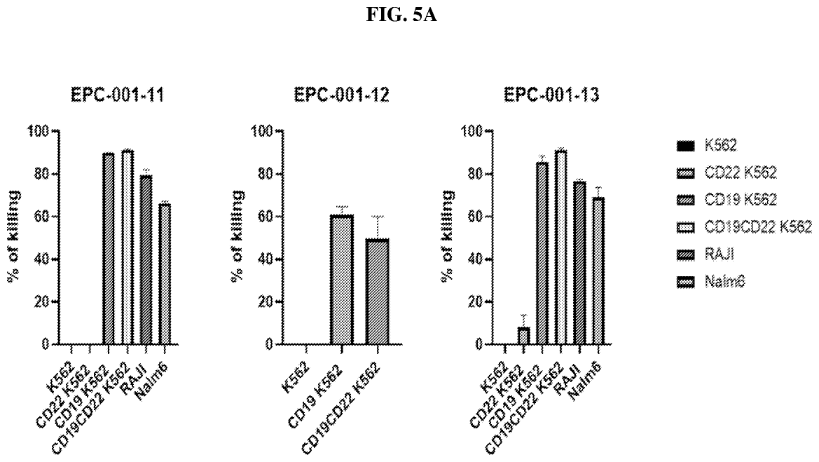

The various CARs showed evenly surface expression pattern on T cells as imaged by Cytation® 5, using both the Alexa Fluor® 647 labeled anti-EGFR antibody and the Alexa Fluor® 647 labeled CD22-Fc fusion protein. See FIGS. 4A and 4B, using EPC-001-19 as an example. Similar pattern observed with different CARs.

### Example 5

### Cytotoxic T Lymphocyte (CTL) Activity of T Cells Expressing Anti-CD19/CD22 Bispecific CAR

Human PBMCs and Pan T cell isolation, virus transduction and T cell expansion were described above. To screen different CAR activity, real time image-based CTL activity assay was performed with target cells engineered with GFP. Briefly, 20,000 of CAR transduced T cells were incubated with 20,000 K562-GFP, CD19/K562-GFP, CD22/K562-GFP, CD19/CD22/K562-GFP, Raji-GFP and Nalm-6-GFP cells respectively and at effector to target cell ratio of 1:1 in RPMI media with 10% FBS. No cytokines were added. The assay was run for 96 hours and GFP of target cells was imaged and quantified by Cytation® 5 scanner. IFNγ was detected with Human IFNγ DuoSet® ELISA kit (R&D System) post CTL assay. Briefly, supernatant was collected after CTL assay terminated at 96 hour. Recombinant IFNγ was serial diluted and included in the assay to create standard curve. Supernatant IFNγ and recombinant IFNγ were assayed following the manufacture's protocol provided. The data was analyzed using GraphPad Prism® 8.0 software.

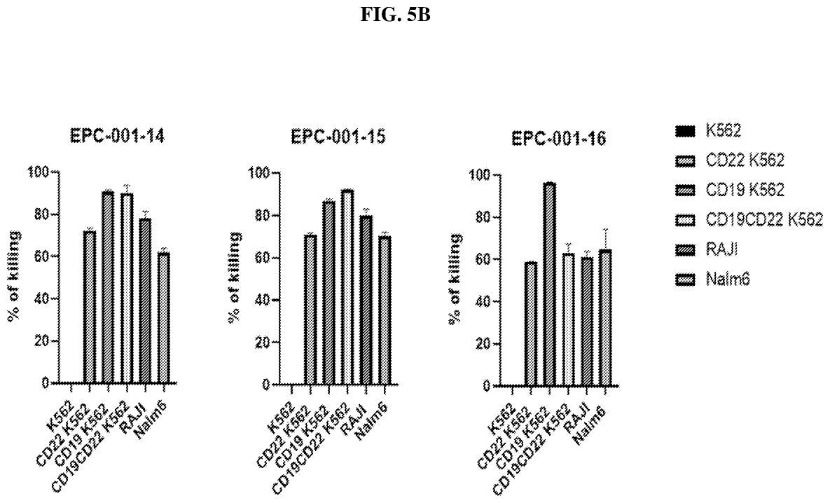

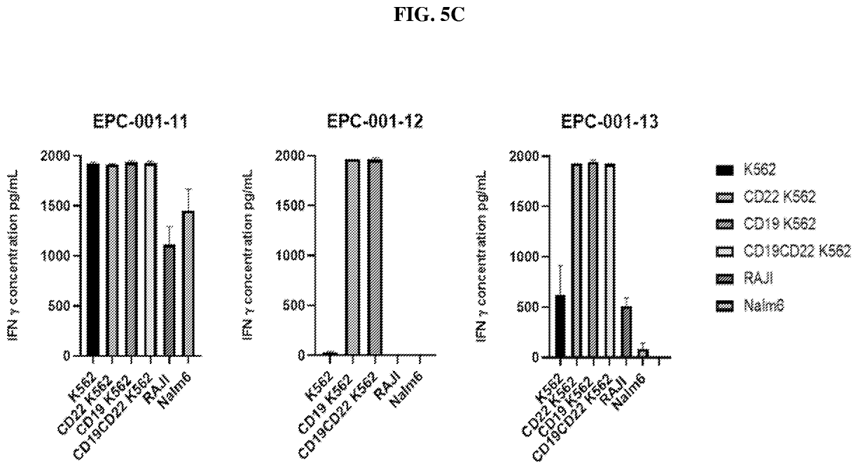

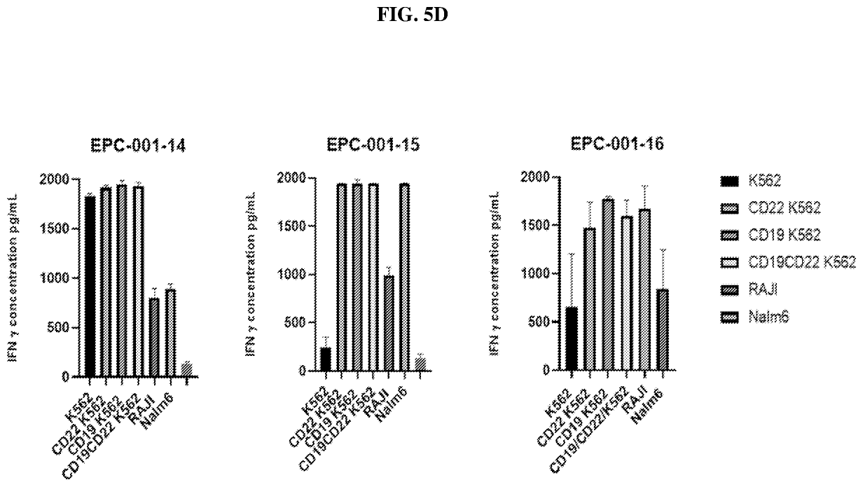

Real-time CTL activities of the CAR-expressing cells against different target cells (including K562, CD22 K562, CD19 K562, CD19/Cd22 K562, Raji cells, and Nalm6 cells) were monitored and the end point of the CTL activities of different CAR constructs (see Examples above) were calculated. A shown in FIGS. 5A-5D, the level of IFNγ secretion correlated with CTL activity as observed in various CARs tested. Multiple donors have been screened with different CARs and showed similar results.

### Example 6

### CTL Assay of EPC-001-16 with Different Effector-to-Target Cell Ratios

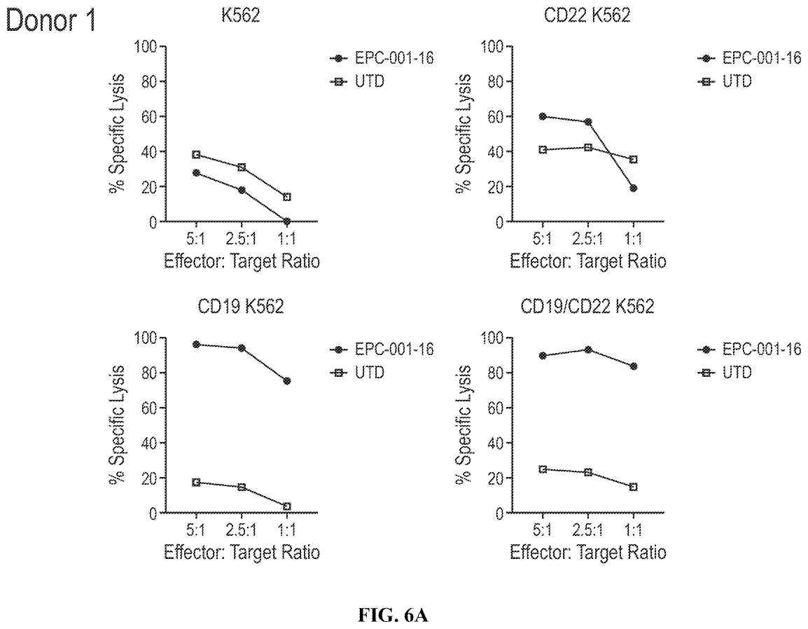

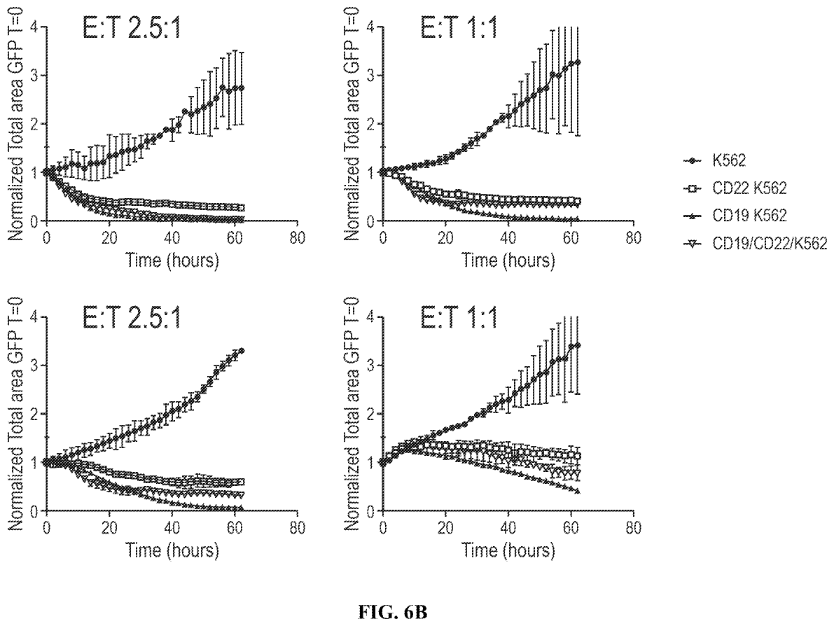

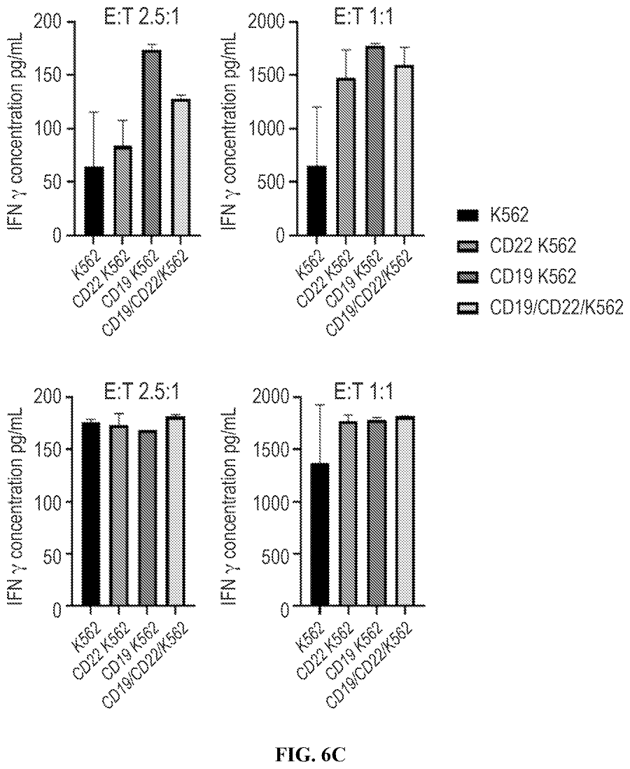

To further evaluate the CTL activity of CAR-T cells, multiple donors were transduced with lentivirus carrying EPC-001-16 (used as a representative anti-CD19/CD22 bispecific CAR) and the resultant CAR-T cells were expanded as described above. For cells derived from Donor 1, the transduced or non-transduced T cells were co-cultured with K562, CD19 K562, CD22 K562 and CD19/CD22 K562 GFP cells at effector to target cell ratio of 5:1, 2.5:1 and 1:1 for 96 hours. The dose dependent target specific CTL activity was observed as shown in FIG. 6A. For cells derived from Donor 2 and Donor 3, the tested effector to target cell ratios were 2.5:1 and 1:1. Similar results were observed as shown in FIG. 6B. The CTL activity of the CAR-T cells against the CD22/K562-GFP cells was lower than that against the CD19/K562-GFP cells. This may result from the lower copy number of CD22 in CD22 K562, which is 5-10-fold lower than the copy number of CD19 in CD19/K562 and CD19/CD22 K562 cells. The difference in engineered GFP expression in individual target cell lines may also play a role. The IFNγ secretion results are shown in FIG. 6C.

### Example 7

### Mechanism Study of CAR-T Cells Expressing Anti-CD19/CD22 Bispecific CARs by CTL Assay

To study the mechanism of individual scFv-based CAR activities, the CD19 and CD22 scFv monospecific CARs and the corresponding bispecific CAR were constructed (see Table 9 below).

TABLE 9
Mono-specific and Bi-specific CAR Constructs

| | scFv Orientation | Linker | EGFRt |
|---|---|---|---|
| CAR No | (N terminus to C terminus | length (AAs) | Inclusion |
| EPC-001-16 | CD22-CD19 | 18 | Yes |
| EPC-001-17 | CD22 | 18 | Yes |
| EPC-001-18 | CD19 | 18 | Yes |

PBMCs and Pan T cells were isolated from 2 donors and transduced with lentivirus carrying EPC-001-16, EPC-001-1-17 or EPC-001-18. The resultant transduced cells were expanded as described and then co-cultured with K562, CD19 K562, CD22 K562 or CD19/CD22 K562 GFP cells at effector to target cell ratio of 2.5:1 and 1:1 for 60 hours and GFP quantified by imaging every 2 hours on Cytation® 5. The EPC-001-16 CD19/CD22 bispecific CAR showed similar CTL activity as compared with CD19 monospecific CAR and CD22 monospecific CAR against all tested target cell lines. FIG. 7. showed CTL cell killing results of various anti-CD19/CD22 bispecific CAR or anti-CD19, anti-CD22 monospecific CARs against K562 cells, CD22 K562 cells, CD19 K562 cells, and CD19/CD22 K562 cells at different E:T ratios.

### Example 8

### CTL Activity of Exemplary Bispecific CAR with Different Linkers

Example To further test whether the different linkers between two different scFvs would affect the CAR activity, CAR constructs having the same anti-CD19 and anti-CD22 scFv fragments as EPC-001-16 and different linkers, i.e., (G₄S)1, (G₄ S)₂ and (G₄S)₃, were constructed. See Table 10 below. Their CTL activities were compared with EPC-001-16.

TABLE 10
CAR Constructs Having Different Linkers

| | scFv Orientation | Linker | |
|---|---|---|---|
| | (N terminus | length | EGFRt |
| CAR No | to C terminus | (AAs) | Inclusion |
| EPC-001-19 | CD22-CD19 | 5 | Yes |
| EPC-001-20 | CD22-CD19 | 10 | Yes |
| EPC-001-21 | CD22-CD19 | 15 | Yes |
| EPC-001-22 | CD22-CD19 | 18 | Yes |
| (Codon optimized | | | |
| EPC-001-16) | | | |

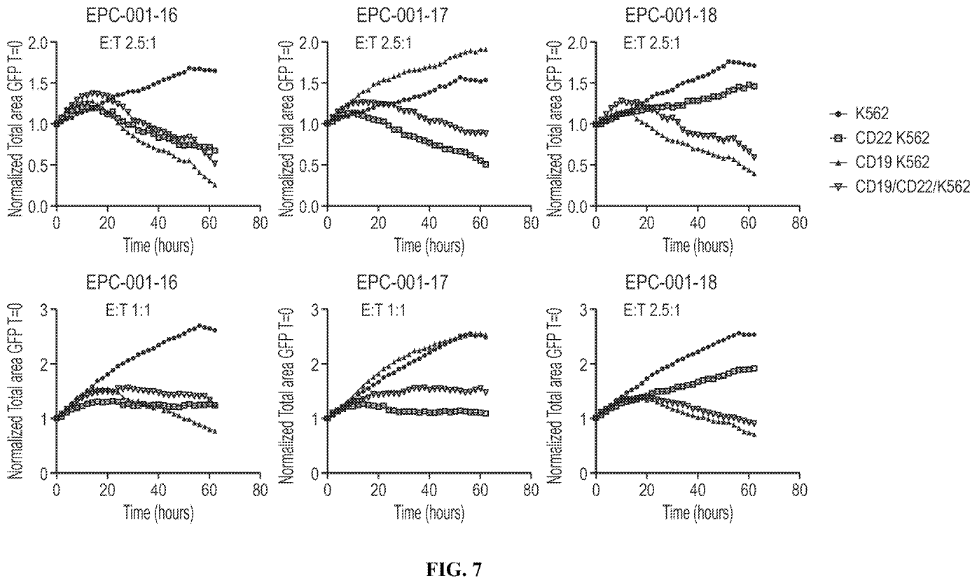

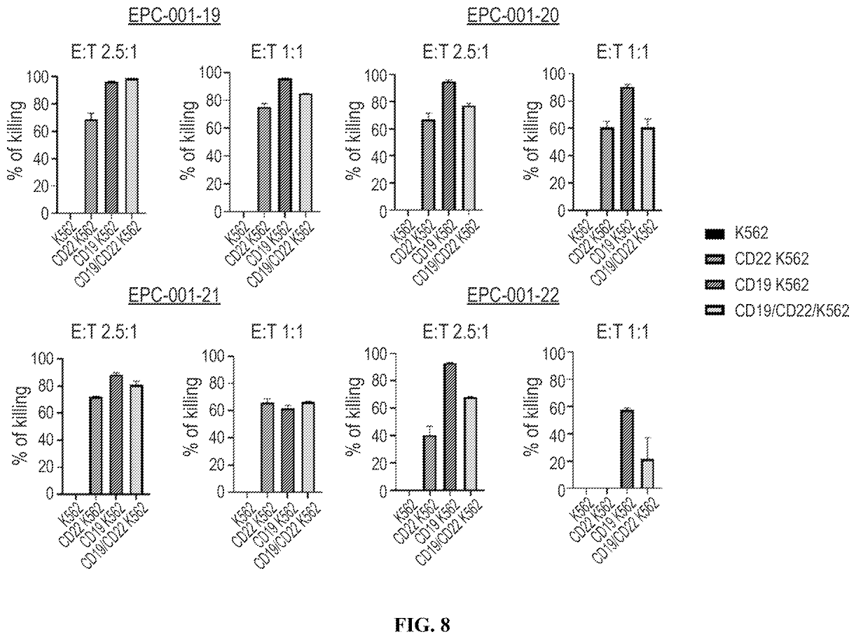

PBMCs and Pan T cells were isolated and transduced with EPC-001-19, EPC-001-1-20, EPC-001-21 and EPC-001-22 lentivirus and expanded as described. The transduced T cells were co-cultured with K562, CD19/K562, CD22/K562 and CD19/CD22/K562 GFP cells at effector to target cell ratio of 1:1 for 60 hours. The CTL activity was imaged every 2 hours by the target cell GFP level on Cytation® 5. The percentage of CTL activity was analyzed using GraphPad Prism® 8.0. The EPC-001-19 showed better CTL activity compared to EPC-001-20, EPC-001-21 and EPC-001-22 as shown in FIG. 8.

### Example 9

### Cytokine Analysis of Exemplary Bispecific CAR by FACS Following CTL Assay

Example To further test cytokine profile of CAR-T cells expressing anti-CD19/CD22 bispecific CAR as disclosed herein upon target cell engagement, EPC-001-19 (as a representative bispecific CAR) was transduced in Pan T cells and expanded. The transduced T cells were co-cultured with K562, CD19/K562, CD22/K562, CD19/CD22/K562, Raji, or Nalm-6 GFP cells at effector to target cell ratio of 5:1 for 96 hours. The IFNγ and Granzyme were detected by intracellular staining on FACS. Briefly, 4 hours before harvesting samples, cells were Golgi blocked using Cell Activation Cocktail with Bredfeldin A following manufacture's recommendation (Biolegend). Cells were spun down at 1,300 rpm for 5 minutes at room temperature. Cells were washed once with 1×PBS then stained with Zombie Aqua™ fixable cell viability dye (Biolegend) at 1:1000 dilution in 1×PBS with 5 minutes incubation at room temperature. Cells were then washed twice with 1×PBS. Next, cells were stained with anti-CD3, anti-CD4, anti-CD8, anti-CD19, anti-CD22, CD22-Fc for 30 minutes at room temperature in dark. Cells were then fixed using eBioscience Foxp3/Transcription Factor Fixation/Permeabilization kit (Thermo Fisher scientific) for 20 minutes in the dark at room temperature. Then, cells were washed twice with 1×PBS and resuspended in 1×PBS for storage at 4 C overnight. Next day, cells were permeabilized using permeabilization buffer (Thermo Fisher Scientific) for 15 minutes in the dark at room temperature. Then, cells were stained for IFNγ and Granzyme intracellular proteins for 30 minutes at room temperature in dark. Cells were washed twice with 1×permeabilization buffer then resuspended in 1×PBS and read on Attune™NxT Flow Cytometer.

At 96 hours, the CD8+ T cells demonstrate specific secretion of both IFNγ and granzyme upon incubating with target cells, ranging from 40-80% of the CD8+ population. Good correlation observed with IFNγ and Granzyme positive CD8 T cells as shown in Table 11 below. Without being bound by theory, the difference observed among different cell lines maybe due to the variation of target expression level in different cell lines.

TABLE 11
Levels of IFNγ and Granzyme B in
Co-Culture of CAR-T Cells and Target Cells

| | IFNγ/ | IFNγ/ |
|---|---|---|
| | Granzyme B Double | Granzyme B Double |
| | Positive Cell % (CD4+) | Positive Cell % (CD8+) |
| EPC-001-19 + | 0.82 | 43.1 |
| K562 | | |
| EPC-001-19 + | 10.9 | 82.0 |
| CD19/K562 | | |
| EPC-001-19 + | 0.92 | 68.5 |
| CD22/K652 | | |
| EPC-001-19 + | 3.01 | 74.9 |
| CD19/CD22/K562 | | |
| EPC-001-19 only | 0.23 | 0.42 |
| EPC-001-19 + | 1.29 | 62.8 |
| Raji | | |
| EPC-001-19 + | 0.42 | 39.4 |
| Nalm 6 | | |

### Example 10

### CAR-T Cell Proliferation Upon Target Cell Engagement

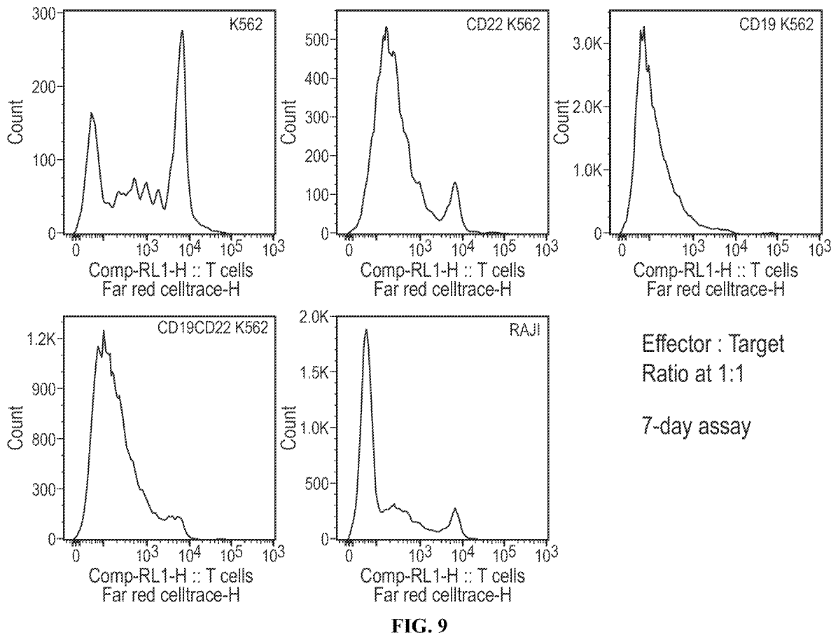

To further test the CAR-T cell expansion upon target cell engagement, 7-day proliferation assay was performed using EPC-001-19 as an example. EPC-001-19 was transduced in Pan T cells and expanded. The transduced T cells were labelled with Cell Trace Far Red at final concentration of 1 uM. 20,000 labeled T cells were co-cultured with 20,000 of K562, CD19/K562, CD22/K562, CD19/CD22/K562 and Raji cells target cells at E:T ratio 1:1 respectively. The assay was set up with RPMI media with 10% FBS and fresh media was added to cells every two days. No cytokine added to the media during the assay. The CAR-T proliferation was analyzed on Attune™ NxT Flow Cytometer. The CAR-T cells demonstrated target cell specific expansion upon engagement over 7 days and correlate with target expression level on cells. FIG. 9. showed the CAR-T cell proliferation upon engagement with target cells at E:T ratio of 1:1.

### Example 11

### CAR-T Cell Persistence Upon Multiple Rounds of Target Cell Challenge

To further examine CAR-T cell persistence, a multiple rounds of target cell challenge experiment was performed using EPC-001-19 as an example. EPC-001-19 was transduced into Pan T cells and expanded. 20,000 of transduced T cells were challenged with 20,000 of K562, CD19/K562, CD22/K562 or CD19/CD22/K562 GFP cells for 48 hours, followed by rechallenge the transduced T cells with fresh 20,000 target cells for another 72 hours, then rechallenge the transduced T cells with fresh 20,000 target cells for additional 72 hours, total of 3 times. The taget cell cell GFP CTL was imaged every 2 hours with Cytation® 5 and quantification was analyzed using GraphPad Prism® 8.0 software. In between each rechallenge, 50 ul of supernatant was collected for cytokine measurement.

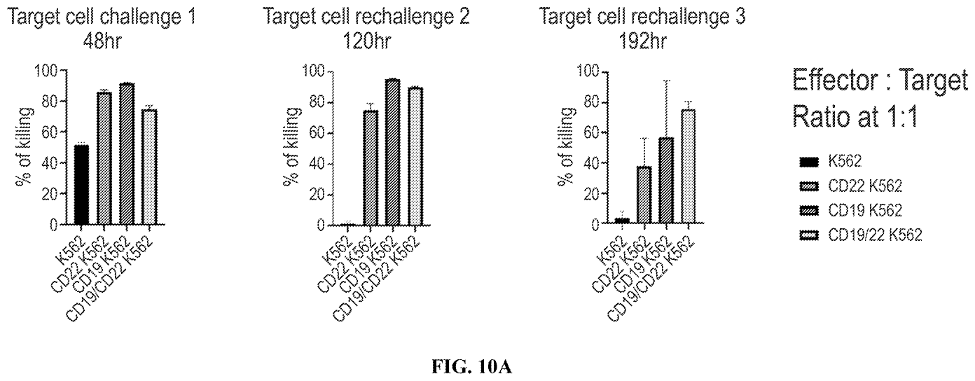

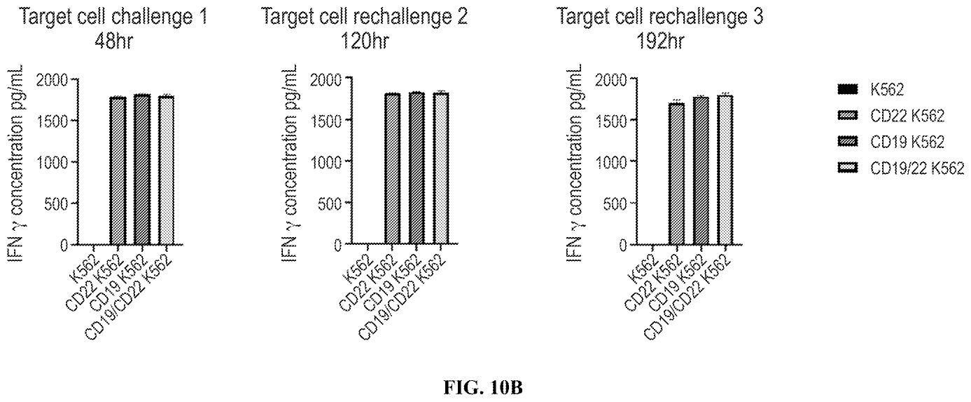

CAR-T cells expressing EPC-001-19 showed persistent CTL activity in 3 rounds of target challenge and rechallenge experiments over 8 days. Similar levels of IFNγ secretion were observed at all time points measured. FIG.10A showed the percentage of cell killing in response to 1, 2, or 3 rounds of target cell challenge. FIG. 10B showed the corresponding IFNγ secretion of CAR-T cells.

### Example 12

### Bispecific CAR Transduced Pan T and Naive T Cell Phenotyping

CAR-T cell phenotype is associated with in vivo T cell persistency. EPC-001-19 was transduced to human Pan T and naïve T cells and T cell phenotype was analyzed using FACS assay with a panel of antibodies detecting T cell differentiation markers. Briefly, anti-CD3, anti-CD4, anti-CD8, anti-CD45RO, anti-CD62L, anti-CCR7, anti-EGFR were used to stain the transduced T cells as described above. Analysis was done by Attune™ NxT software. The CD4 and CD8 positive Tcm and Tem cells were gated and the results showed more Tcm population and less Tem in transduced naïve T cells than in Pan T cells.

### Example 13

### In Vivo Anti-Tumor Effects Using Disseminated Raji-Luciferase Model

Example In vivo anti-tumor efficacy of EPC-001-23 CAR-T was evaluated in a disseminated Raji cell model in NCG mice. EPC-001-23 CAR was generated by removing of EGFRt from EPC-001-19. EPC-001-23 was transduced in naïve Pan T cells and expand in vitro for 4 days. 1e6 of Raji-luciferase cells were inoculated to NCG mice. At day 3, PBS control (Group 1), 0.125e6 (Group 2) and 0.25e6 (Group 3) EPC-001-23 CAR-T cells were dosed to the mice. The mice were imaged every 2-3 days and body weight were measured. At day 10, 19 and 33, blood was taken from mice. Spleen was also collected at day 33. CAR-T cell phenotype was analyzed by FACS assay immediately after blood taken and tissue collection.

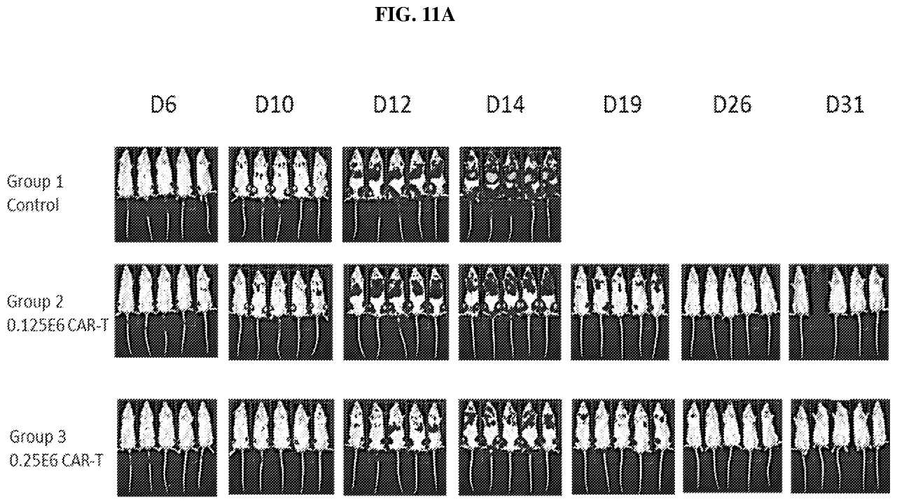

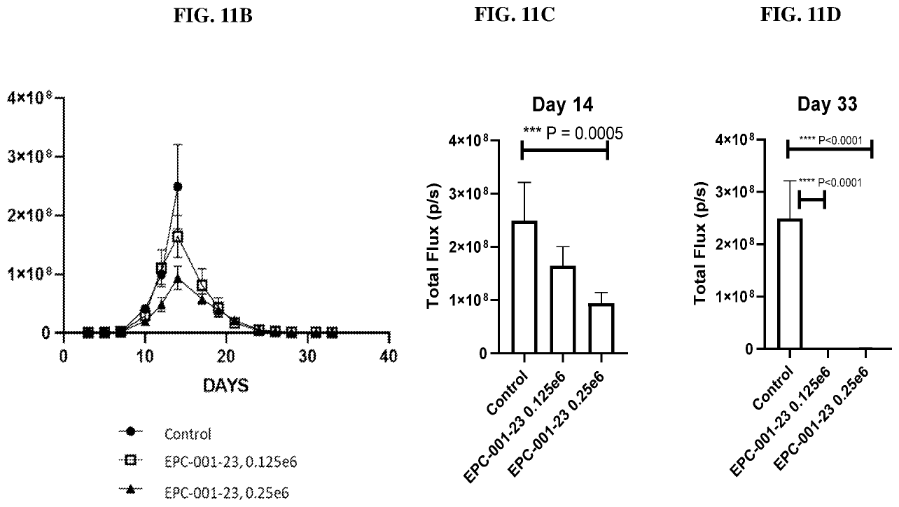

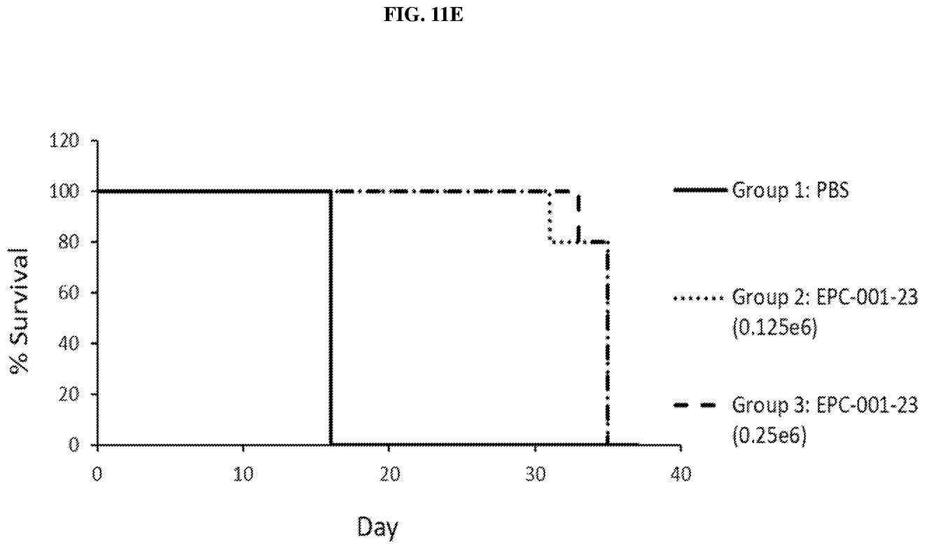

As indicated in FIGS. 11A-11B, dose-dependent tumor growth inhibition was observed at days 6-14. The Group 3 animals showed significant tumor growth inhibition than the control group. Cancer cells were eradicated from mice treated with the CAR-T cells at both doses (Group 2 and Group 3 mice) at day 31. FIGS. 11C-11D: charts showing tumor cell luciferase quantification on Day 14 and Day 33, respectively, after treatment.

The control mice were euthanized at day 14 due to the overgrowth of cancer cells. One mouse from low and high dose CAR-T treatment group was euthanized at Day 31 and Day 33 due to GVHD respectively as shown in FIG. 11E. No significant body mouse weight change over the treatment course.

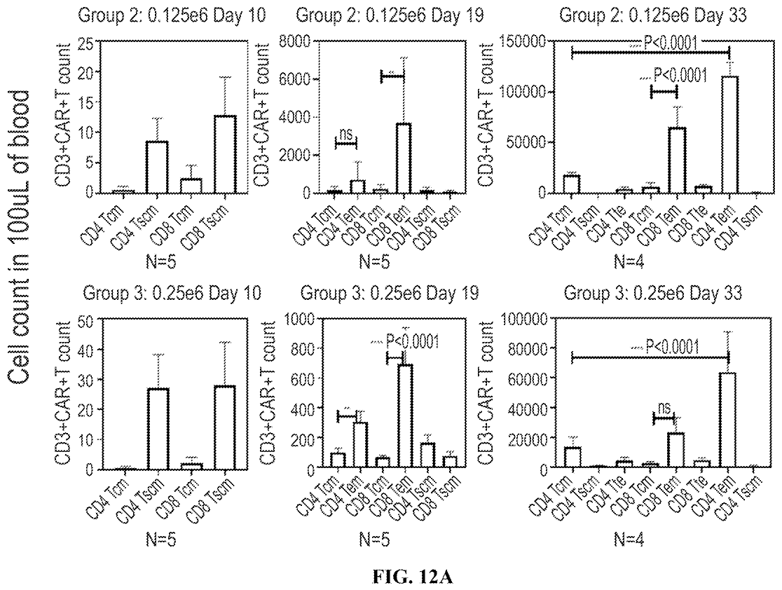

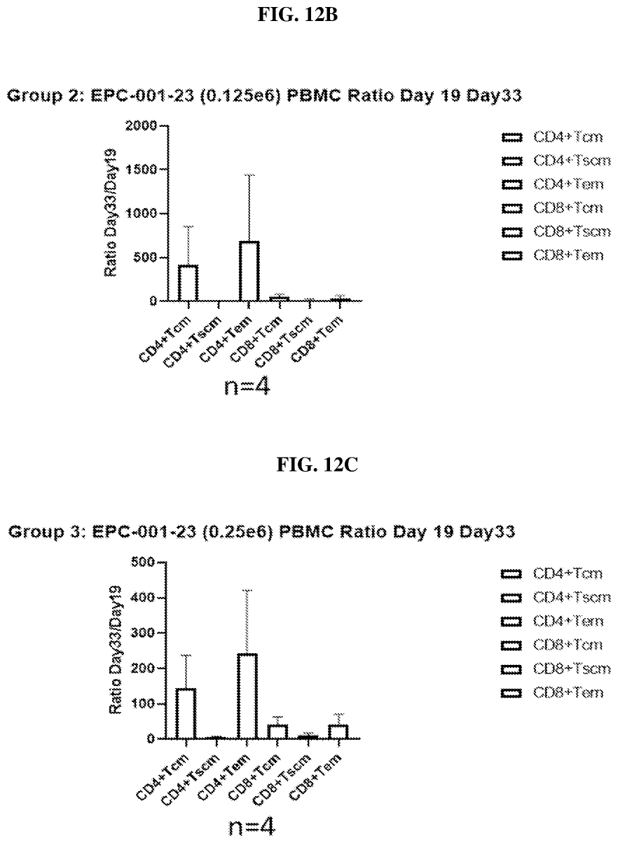

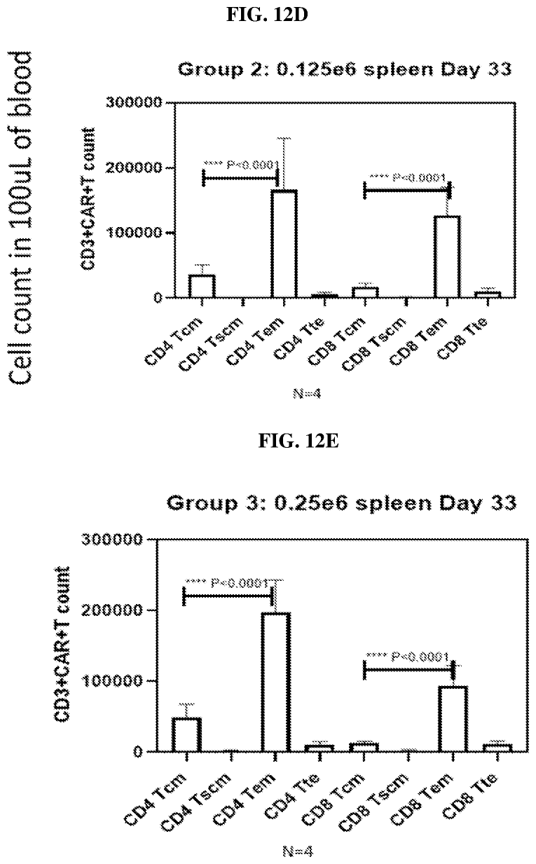

Using the EPC-001-23 construct as an example, CAR-T cell expansion and persistency were demonstrated over the treatment course, as shown in FIGS. 12A-12C. CAR-T cells differentiated and expanded from Tscm to Tcm and Tem over treatment course. In addition, both CD4 and CD8 Tcm and Tem of EPC-001-23 CAR-T cells were found to be homed into spleen.

FIGS. 12D-12E showed cell counts in spleen on Day 33 of Group 2 and Group 3 mice, respectively.

### Example 14

### Characterization of Anti CD22-CD19 Bispecific Antibodies

This example describes characterization of exemplary anti-CD19/CD22 bispecific antibodies in scFv-Fc fusion format.

(a) CD19-CD22 Bispecific scFv Preparation

The anti-CD19scFv-Fc, anti-CD22scFv-Fc and Anti-CD22scFv-CD19scFv-Fc antibodies were expressed transiently in Expi293F™ cells in free style system (Invitrogen) according to standard protocol. The cells were grown for five days before harvesting. The supernatant was collected by centrifugation and filtered through a 0.2 ittm Polyether sulfone (PES) membrane. The fusion protein was purified by MabSelect™ PrismA protein A resin (GE Health). The protein was eluted with 100 mM Glycine pH2.5+150 mM NaCl and quickly neutralized with 20 mM citrate pH 5.0+300 mM NaCl. The antibody was then further purified by a Superdex® 200 16/600 column. The monomeric peak fractions were pooled and concentrated. The final purified protein has endotoxin of lower than 10 EU/mg and kept in 20 mM Histidine pH 6.0+150 mM NaCl.

### (b) Binding Activity of Anti-CD19, Anti-CD22, and Anti- CD19/CD22 Bispecific Antibodies

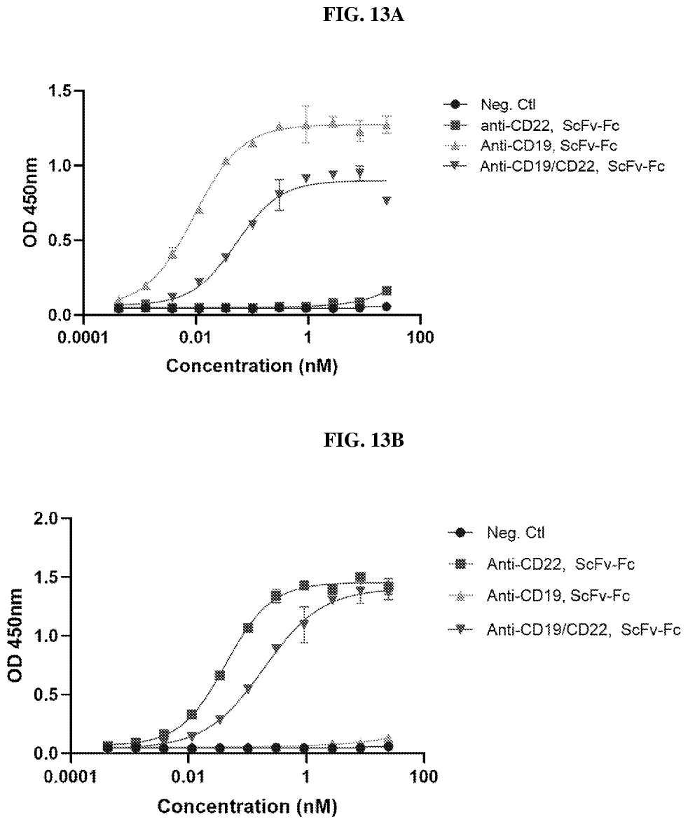

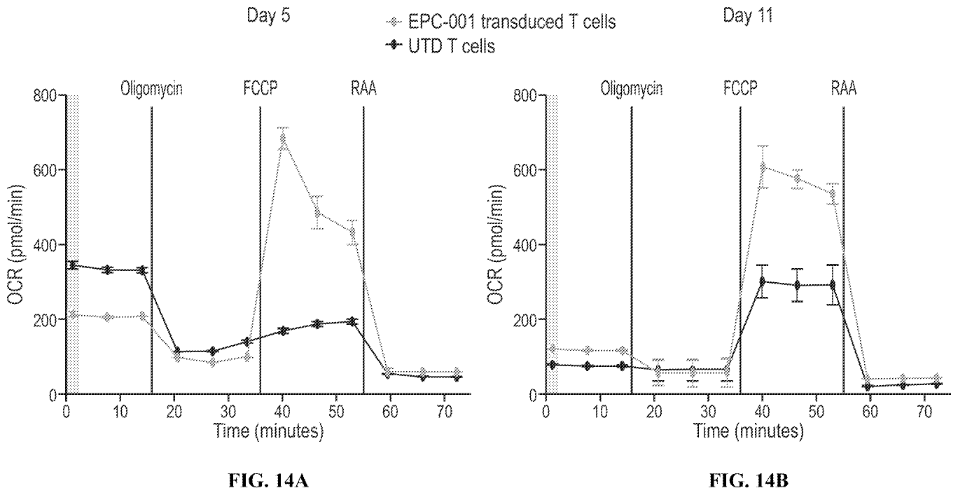

An ELISA assay was developed to determine the EC50 for anti-CD19-Fc, Anti-CD22-Fc and Anti-CD22-CD19-Fc fusion proteins. Briefly, 384 well plate was immobilized with HIS tagged human CD19 or CD22 recombinant protein at final concentration of 2 μg/mL in 1×PBS in total volume of 25μL per well. The plate was incubated overnight at 4° C. followed by blocking with 80μL of superblock per well for 1 hour. Purified anti-CD19, CD22 or CD22-CD19 scFv Fc fusion proteins were 2-fold serial diluted starting at 25 nM, 25 μL was added to human CD19 or CD22 immobilized wells and incubated for 1 hour with shaking. CD19 or CD22 binding was detected by adding 25 μL of anti-hFc HRP diluted at 1:5000 in 1×PBST. In between each step, the plate was washed 3 times with 1×PBST in a plate washer. The plate was then developed with 20 μL of TMB substrate for 5 mins and stopped by adding 20 μL of 2 N sulfuric acid. The plate was read at OD450 nm (BIOTEK plate reader) and data plotted using GraphPad Prism® 8.1 software. The results of ELISA binding are shown in FIGS. 13A-13B. EC₅₀ values for anti-CD19 or CD22 scFv-Fc to CD19 and CD22 are shown in. Table 12.

TABLE 12
EC50 of monospecific and bispecific scFv-Fc
binding to CD19 or CD22 targets by ELISA

| EC50 (nM) | CD19 Binding | CD22 Binding |
|---|---|---|
| Anti-CD22, ScFv-Fc | — | 0.043 |
| Anti-CD19, ScFv-Fc | 0.0094 | — |
| Anti-CD19/CD22, ScFv-Fc | 0.052 | 0.189 |

(c) Binding of Anti-CD19/CD22 scFv Bispecific Antibodies to Endogenous Cell Lines by FACS Analysis

To determine the specific target cell binding activity of antibodies in scFv format and bispecific scFv-Fc fusion format, anti-CD19 or anti-CD22 monospecific scFv and anti-CD19/CD22 bispecific scFv-Fc fusion antibodies were tested in FACS binding assays with CD19 and CD22 expressing cell lines Raji and Nalm-6, as well as the negative cell line U87MG. Briefly, each scFv-Fc fusion was diluted to 25 nM and incubated with 100,000 cells of Raji, Nalm 6 and U87MG in 96 wells plate at 4° C. for 1 hour with shaking. Cells were spun down at 1300 rpm for 5 minutes at 4° C. to remove unbound antibodies. Cells were then washed once with 200 μL of PBS per well. Samples were mixed with an Alexa Fluor® 647-conjugated anti-hFc antibody (secondary antibody, 100 μL, 1:1000 diluted) and incubated at 4° C. for 30 minutes in dark with shaking. Samples were then spun down at 1300 rpm for 5 minutes at 4° C. and washed twice with 200 μL of 1×PBS per well. The resultant samples were reconstituted in 200 μL of 1×PBS and read on Attune™ NxT Flow Cytometer. Analysis was done by counting only Alexa Fluor® 647-positive cells and then plotting using GraphPad Prism® 8.1 software.

As observed in FACS analysis, all Anti-CD19, Anti-CD22, and Anti- CD19/CD22 antibodies in Fc-fusion format showed specific binding activity to CD19 and CD22 expressing Raji and Nalm 6cell lines, but not to U87MG and U251MG cells, which do not express CD19 and CD22.

(d) Binding kinetics of Anti-CD19/CD22 Bispecific Antibodies, by Surface Plasmon Resonance (SPR)

Kinetic analysis of anti-CD19, anti-CD22, and anti-CD22/CD19 scFv-Fc fusion were assessed by SPR technology with Biacore™ T200. The assay was run using Biacore™ T200 control software version 2.0. For each cycle, 1 μg/mL of Fc-fusion protein was captured for 60 seconds at flow rate of 10 ul/min on flow cell 2 in 1×HBSP buffer on Protein A sensor chip. 2-fold serial human CD19 or CD22-HIS tagged protein was injected onto both reference flow cell 1 and Fc fusion protein captured flow cell 2 for 150 seconds at flow rate of 30 μl/mins followed by wash for 300 seconds. The flow cells were then regenerated with Glycine pH2 for 60 seconds at flow rate of 30 ul/mins. 8 concentration points from 100-0 nM was assayed per Fc fusion in a 96 well plate. The kinetics of Anti-CD19, CD22, CD22-CD19 binding to CD19 or CD22 protein was analyzed using Biacore™ T200 evaluation software version 3.0. The specific binding response unit was derived from subtraction of binding to reference flow cell-1 from Fc fusion protein captured flow cell-2. Table 13 below shows the binding kinetics of the anti-CD19, CD22 and CD22-CD19 scFv-Fc fusion protein to CD19 or CD22.

TABLE 13
Kinetics of Monospecific andBispecific Antibodies
to CD19 or CD22 target Antigens

| Sample ID | Protein | Ka(1/Ms) | kd(1/s) | KD(M) |
|---|---|---|---|---|
| anti-CD22, | rhCD19 | N/A | N/A | N/A |
| ScFv-Fc | rhCD22 | 1.22E+05 | 1.77E−04 | 1.44E−09 |
| anti-CD19, | rhCD19 | 5.94E+04 | 2.23E−04 | 3.75E−09 |
| ScFv-Fc | rhCD22 | N/A | N/A | N/A |
| Anti-CD19/CD22, | rhCD19 | 5.72E+04 | 2.50E−04 | 4.37E−09 |
| ScFv-Fc | rhCD22 | 7.90E+04 | 2.66E−04 | 3.37E−09 |
| FMC63 IgG | rhCD19 | 8.71E+04 | 7.64E−04 | 8.77E−09 |
| | rhCD22 | N/A | N/A | N/A |

### Example 15

### Evaluation of CAR-T Metabolism from CAR-T Production

To predict in vivo CAR-T persistency, a CAR-T metabolic assay that evaluated mitochondria oxygen consumption was developed. EPC-001-23 was transduced to activated human naïve T cells and expanded for up to 11 days. At day 5 and day 11, CAR-T cells and non-transduced T cells were tested in the assay using Agilent Seahorse® instrument.

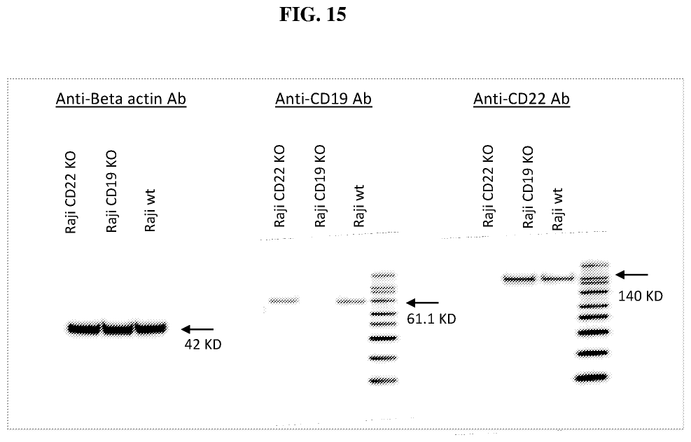

Following manufacture's protocol, 250,000 CAR-T cells were plated on poly-D-Lysine treated plate. First, 1.5 μM of Oligomycin was added to inhibit mitochondria ATP synthase in port A. Next, 1 μM of Carbonyl cyanide-4 (trifluoromethyoxy) phenylhydrazone (FCCP) was added in port B. Finally, to block mitochondrial respiration 0.5 μM Rotenone and Antimycin were added together in port C to inhibit both complex I and III of the Electron Transport Chain. In between each drug addition, samples were mixed three times and read three times. Plate was read on an Agilent Seahorse® XFe96 instruments using the XF Mito Stress program. Analysis was done using Agilent Seahorse® analyzer software. FIGS. 15A-15B shows that the CAR candidate EPC-001-023 demonstrated 2-3-fold more oxygen consumption at day5, which continued to day 11.

### Example 16

### CD19 or CD22 Raji-Luc KO Cell Line Generated by CRISPR Technology

This example evaluates the mechanism underlying the ability of EPC-001-023 CAR-T cells to overcome escape of CD19 or CD22 expressing targets.

### (a) CRISPR Cell Line Generation

To identify the mechanism of EPC-001-023 CAR-T mediated effects, CD19 or CD22 knock out cell lines produced by the CRISPR technology and animal models were developed. Briefly, CRISPR sgRNA sequences were designed using the CRISPick database from Broad Institute. Top 5 selective sgRNA sequences targeting CD19 and CD22 were designed (Table 14).

TABLE 14
sgRNA sequences for CD19 and CD22 targets

| Target | sgRNA | SEQ ID NO |
|---|---|---|
| CD19 | CTAGGTCCGAAACATTCCAC | 69 |
| CD19 | GGAAAGTATTATTGTCACCG | 70 |
| CD19 | GCAATGACTTAGGCCCCTTG | 71 |
| CD19 | AAGATGAAGAATGCCCACAA | 72 |
| CD19 | ATGAAAAGCCAGATGGCCAG | 73 |
| CD22 | ATTCATACCGGGTAACACTG | 74 |
| CD22 | AAGACTCTATGAAAGCACAA | 75 |
| CD22 | CTCTTCCAACAAATTACACG | 76 |
| CD22 | AAACCTGCGCGAAGTGACCA | 77 |
| CD22 | TTCCCATGGTGACTCCACTG | 78 |

sgRNA were synthesized by Integrated DNA technologies and cloned into lentiCRISPRv2 vector using enzyme digestion method. Plasmid sequences were confirmed. Lentiviruses for these constructs were made using Expi293™ cells transfected with polyethylenimine (PEI). Viruses were concentrated using ultracentrifugation method and then directly transduced into Raji Luciferase cells followed by 2 hours spinoculation at room temperature. After 2 days, cells were stained with commercial anti-human CD19 Alexa Fluor® 647 or anti-human CD22 Alexa Fluor® 647 followed by single cell sorting for CD19 or CD22 knockout expressions. Cells were maintained in culture for 1 month under drug selection pressure. Cells were characterized by flow cytometry and Western blot for CD19 and CD22 expression.

### (b) Confirmation of CD19 or CD22 Knock Out Cell Lines by FACS Assay

The CD19 and CD22 knock out single clones were screened by FACS. Briefly, 100,000 cells of each clone were plated in 96 wells plate at 4° C. for 1 hour with shaking. 25 nM of anti-human CD19-FITC or anti-human CD22-Alexa Fluor® 647 were incubated with cells in final volume of 100 μL at 4 C for 1 hr. Cells were spun down at 1300 rpm for 5 minutes at 4° C. to remove unbound antibodies. Cells were then washed twice with 200 μL of PBS per well. The resultant samples were reconstituted in 200 μL of 1×PBS and read on Attune™ N xT T Flow Cytometer. Analysis was done by counting only Alexa Fluor® 647-positive cells or FITC positive cells and then plotted using GraphPad Prism® 8.1 software.

As determined by FACS analysis, the CD22 knock out cells showed complete loss of CD22 expression but full expression of CD19 as compared to the parental Raji cells. Similarly, the CD19 knock out cells showed complete loss of CD19 expression but full expression of CD22 as compared to the parent Raji cells.

### (c) Confirmation of CD19 or CD22 Knock Out Cell Lines by Western Blot Assay

The CD19 or CD22 knock out cell lines were further confirmed by Western Blot. Briefly, for each cell line, 1e6 cells were lysed with 100 μL of 1×cell lysis buffer (Cell Signaling Cat# 9803) containing PMSF and protease inhibitor cocktail (Cell Signaling Cat#5871). Samples were incubated on ice for 20 minutes, then spin down at 13,000 rpm for minutes at 4 C. Supernatant were transferred into new tube. 25 μL of whole cell lysate contained SDS loading buffer and p-mercaptoethanol were loaded on 12 well SDS-PAGE gel ran for 22 minutes at constant 200 voltage. Proteins were transferred onto PVDF membrane using iBlot™ 2 according to manufacturer's instruction. The membrane blot was blocked with 5% milk powder in 1×PBST (0.05% polysorbate 20 in 1×PBS) for 1 hour at room temperature. Then washed 3 times with 1×PBST; each wash were 10 minutes at room temperature. Primary were added at to blot at 1:1000 dilutions and incubated at 4° C. overnight. Next day, the blot was washed 3 times with 1×PBST; (10 minutes each at room temperature). Secondary antibody was added at 1:1000 dilution anti-rabbit HRP and incubated at room temperature for 1 hour. The blot was then washed thrice with 1×PBST (10 minutes each at room temperature) and developed using ECL™ reagent (Cell Signaling Cat#6883), followed by reading on a ProteinSimple® Fluorchem E gel imager. As shown in FIG. 15, no CD19 or CD22 protein were detected in the CD19 and CD22 knock out cell lines while parental Raji cell line showed abundant CD19 and CD22 expression.

### (d) Quantification of CD19 and CD22 Receptor Numbers in Knock Out (KO) and Knock-Down (KD) Cell Lines

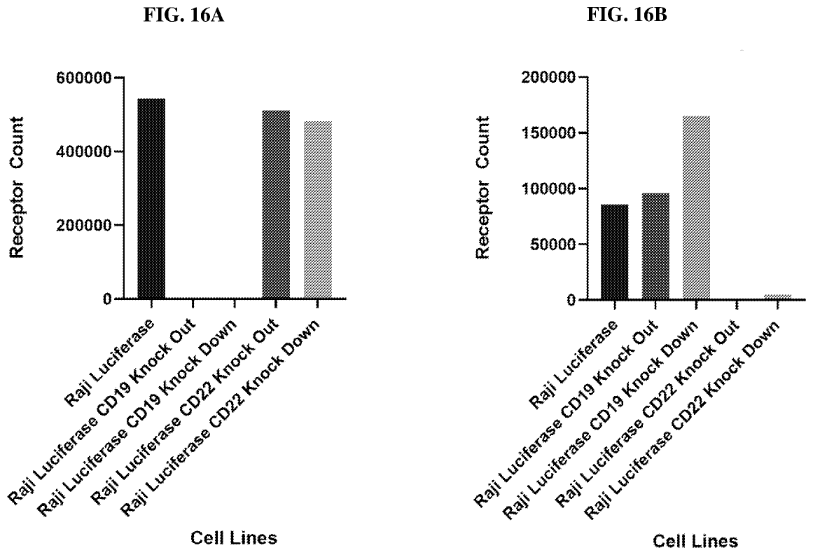

To further characterize the CD19 and CD22 expression levels in knock out cell lines, quantification FACS assay was performed using QUANTUM Alexa Fluor® 647 MESF microsphere beads (Bangs Laboratories Inc) for standard calibration following manufacture's protocol. The parental Raji cell line showed high level of CD19 and CD22 expression. The expression level of CD19 was approximately 5-fold higher than that of CD22 in parental Raji cell line. CD19 and CD22 knock out cell lines showed non-detectable CD19 or CD22 expression. CD19 or CD22 knock down cell lines showed very non-detectable CD19 or very low CD22 copy number. The CD19 and CD22 receptor copy numbers are summarized in Table 15. See also FIGS. 16A-16B.

TABLE 15
CD19 and CD22 Receptor Counts by Q-FACS Assay

| | CD19 Receptor | CD22 Receptor |
|---|---|---|
| Raji Luciferase | 543777 | 85644 |
| CD19 KO | 0 | 95812 |
| CD19 KD | ND | 165024 |
| CD22KO | 481819 | 0 |
| CD22 KD | 511181 | 5279 |

(e) EPC-001-023 Mono and Bispecific scFv-Fc Binding on CD19 or CD22 Knock-Out Cell Lines

Binding of mono and bispecific antibody fragments (in scFv-Fc fusion format) corresponding to the antigen-binding moieties in EPC-001-23 CAR-T cells to Raji parental, CD19 knockout and CD22 knockout cells were tested by FACS as described above. The results show that all scFvs bind to the tested cell lines, while no binding of CD19 scFv and CD22 scFv to Raji CD19 or CD22 knock out cell lines was observed.

### Example 17

### EPC-001-23 in vivo Mechanism Study in Mouse Models Implanted with CD19 or CD22 Knock-Out Raji Cells

This example analyzes the mechanism underlining in vivo functionalities of EPC-001-23 using dual-specific and mono-specific targeting.

A NCG mouse model was used to study the effect of EPC-001-23 CAR-T on disseminated parental Raji cells, and CD19 KO or CD22 KO Raji cells. EPC-001-23 CAR was generated as described above. All Raji cells expressed luciferase for purposes of imaging and quantitation of tumor load.

At day 3, PBS control (Group 1), 0.25e6 EPC-001-23 CAR-T cells and anti-CD19 control CAR-T cells (tisagenlecleucel) were dosed to mice, which were implanted with 0.3e6 of parental Raji. 1e6 EPC-001-23 was dosed to CD19 knock out Raji and CD22 knock Raji cell lines. The mice were imaged every 3-4 days and body weight measured. At day 36, blood was collected, and the spleen resected for analysis of CAR-T cell phenotype by FACS. Control mice were euthanized at day 14 due to the overgrowth of cancer cells.

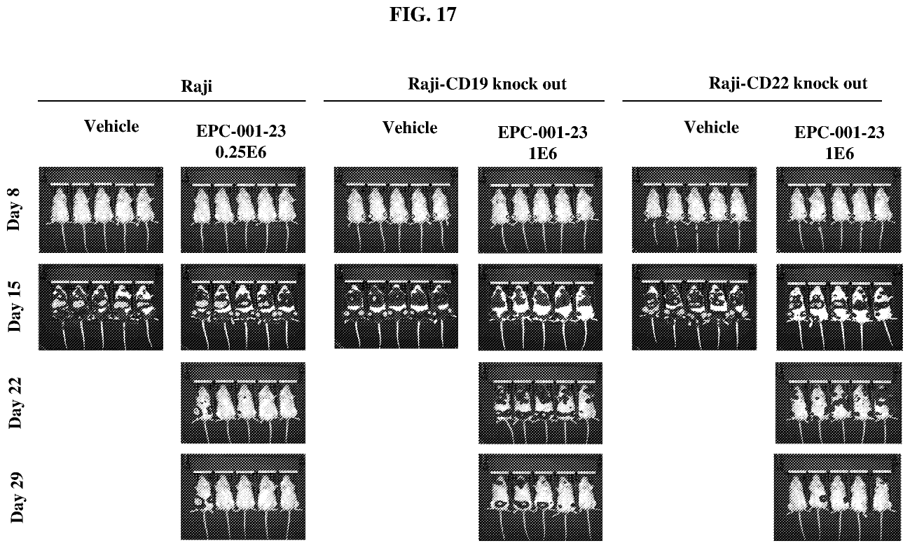

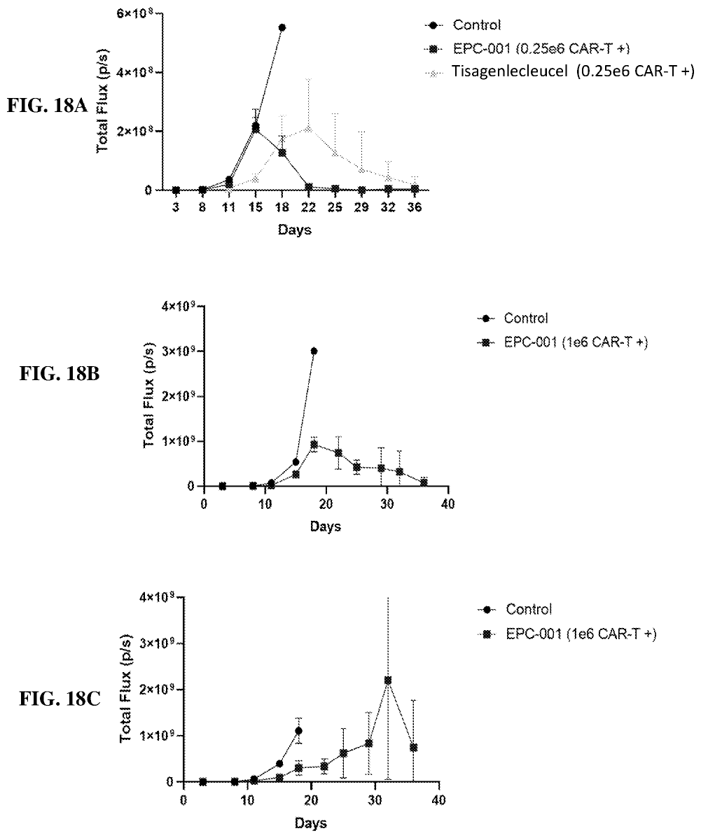

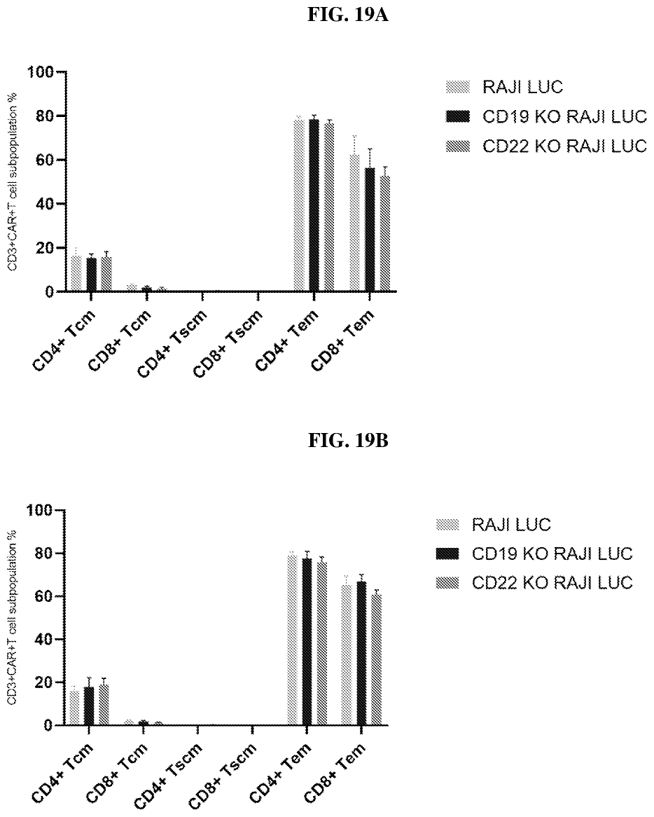

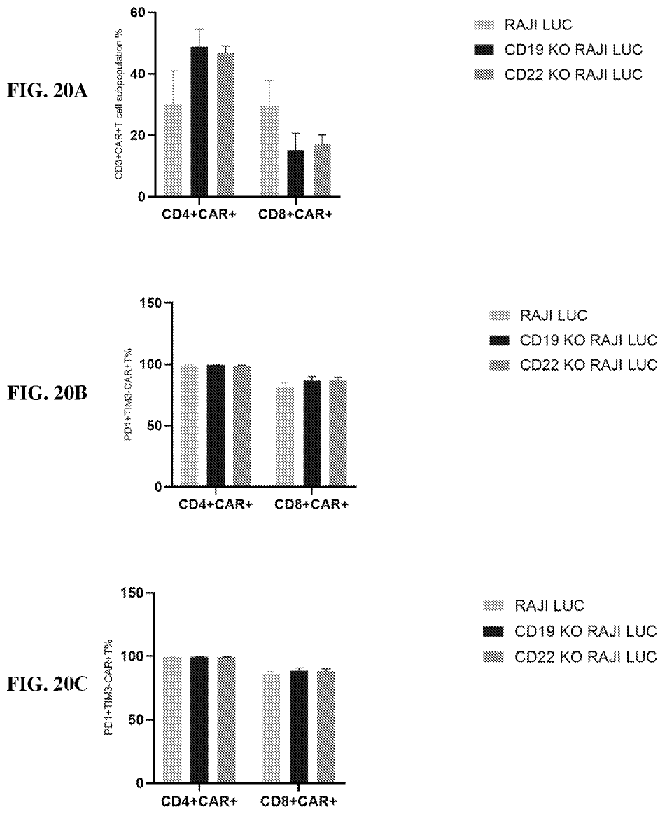

As shown in FIG. 17, tumor growth inhibition was observed in all CAR-T cell treated groups. Parental Raji cells were eradicated from mice treated with the CAR-T cells more robustly by EPC-001-23 CAR-T cells through bispecific targeting of CD19 and CD22. Anti-tumor activity was also observed with CD19 or CD22 knock-out Raji cells through monospecific engagement of the EPC-001-23 CAT cells to the target cells. Quantitative assessment of tumor load is shown in FIGS. 18A-18C. Phenotyping analysis performed on the blood and spleens demonstrated expansion and persistence of the EPC-001-23 CAR-T cell over the course of the treatment (FIGS. 19A-19B), with the CAR-T cells differentiating and expanding to Tcm and Tem in PBMCs, and homing to the spleen. Moreover, both CD4+ and CD8+ CAR-T cells were present at similar proportions in PBMCs and spleen on Day 36 and without exhaustion detected as PD1+, Tim3− CAR+ T cells (FIGS. 20A-20C).

In sum, the results provided in this example show that the anti-CD19/CD22 bi-specific CAR-T cells (using EPC-001-23 as an example) can target not only cancer cells expressing both CD19 and CD22, but also cancer cells expressing only one of the two target antigens. Such a feature is desired in addressing potential targe escape in monospecific CAR-T cell therapy.

### Example 18

### Effects of EPC-001-23 Against CD19 KO or CD22 KO Raji Cells In Vitro and In Vivo

### (a) In Vitro Cytotoxicity

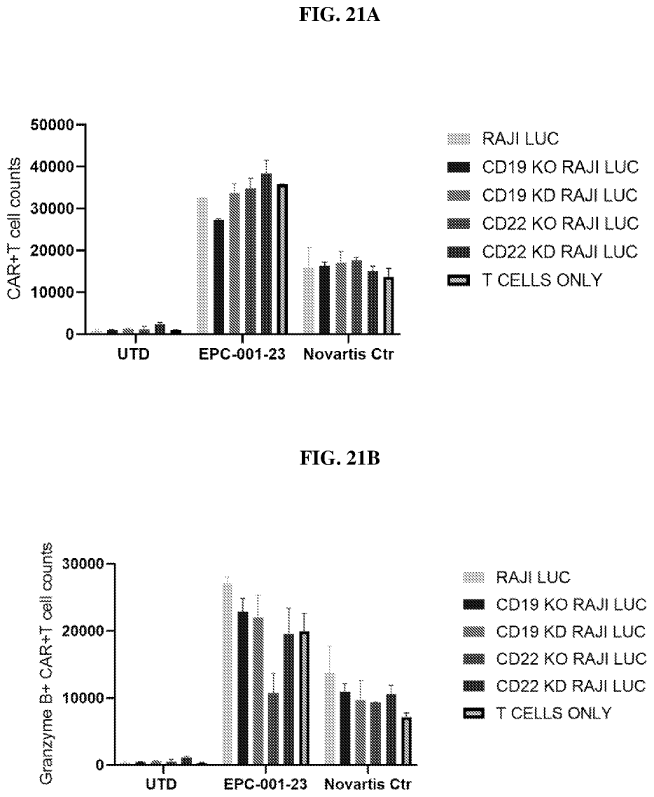

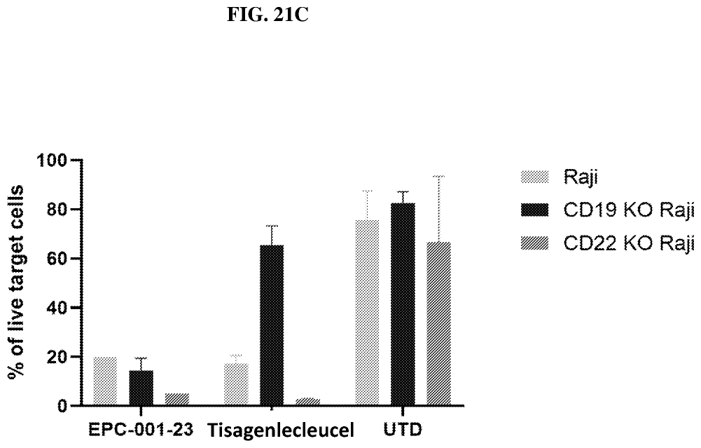

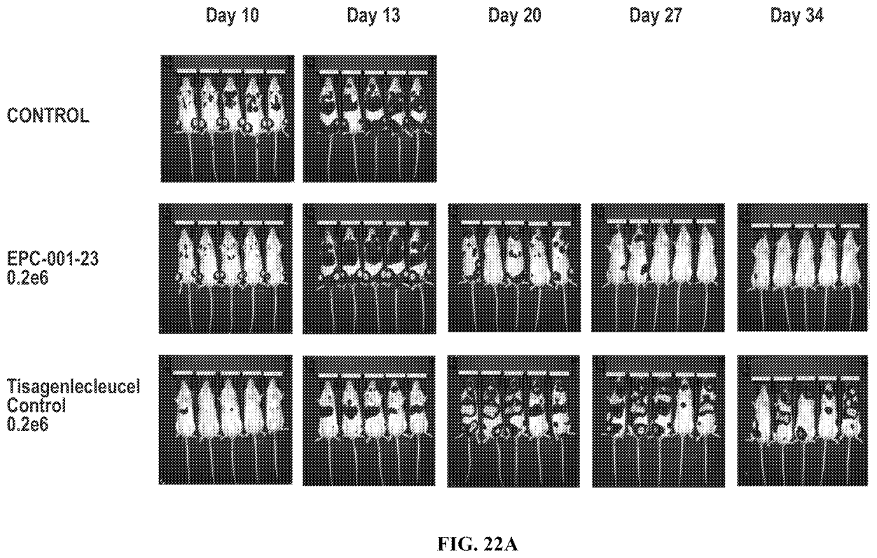

EPC-001-23 and tisagenlecleucel (anti-CD19 CAR-T cells) have been produced as described above. The CAR-T cells were incubated with parental Raji, CD19KO, CD22KO, CD19 knock down (CD19KD), or CD22KD cells at an E:T ratio of 5:1 for 72 hours. FACS assay was used to assess the CAR-T cell expansion and activation by counting the CAR+T cells and granzyme B+ CAR+ T cells, respectively. EPC-001-23 showed more robust CAR-T cell expansion (FIG. 21A) and activation (FIG. 21B) at 72 hours as compared to tisagenlecleucel (used as a control).

Target cell killing activity was also examined in a target cell rechallenging assay as disclosed above. Briefly, the CAR-Ts cells were produced and expanded in vitro. 100,000 of CAR+ T cell were incubated with 5000 target cells at ratio of 20:1 at day 4 post-transduction for stimulation 1 in a 96 well plate in 10% FBS/ RPMI. Samples were spun down at 1,300 rpm for 5 minutes at room temperature then incubated at 37C with 5% CO2 for 72 hours. After 3 days of incubation, the plate spun down at 1,300rpm for 5 minutes. Carefully, removed 50uL of supernatant and discard. For rechallenge 2, added fresh 10,000 targets cells in 50 ul of 10% FBS/RPMI to plate containing CAR-T cells. Spin plate down at 1,300rpm for 5 minutes, followed by incubate at 37 C with 5% CO2 for 72 hours. Repeat the rechallenge 3 times as described. After a final 72-hour incubation, target cells were counted by were staining with 5 uL of anti-human CD19 AF647 (Biolegend, Cat# SJ25C1) and 5 uL of anti-human CD22 AF647 (Biolegend, Cat# HIB22) as described above. Stained cells were resuspended in 200 uL of 1×PBS read on Attune™ 3 lasers flow cytometry (Thermo Fisher scientific). Analysis was done on Attune™ software and GraphPad Prism® 8.

As known in FIG. 21C, EPC-001-23 and tisagenlecleucel showed similar target cell killing activity in the presence of a low level of live target cells, including parental Raji cells and CD22 KO Raji cells. On the other hand, EPC-001-23 showed much stronger target cell killing activity against CD19 KO Raji cells as compared to tisagenlecleucel.

### (b) In Vivo Cytotoxicity

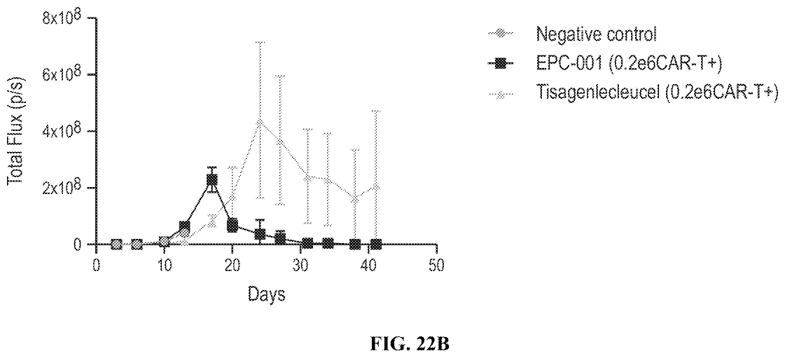

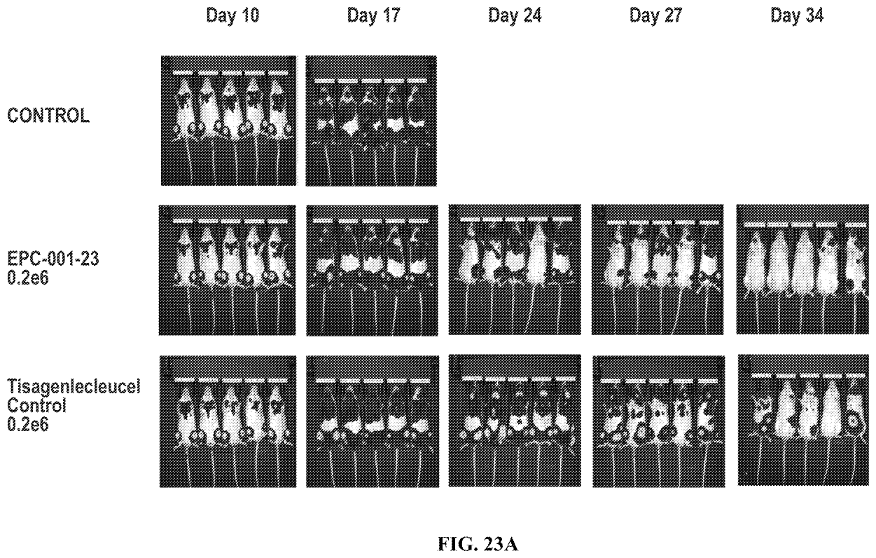

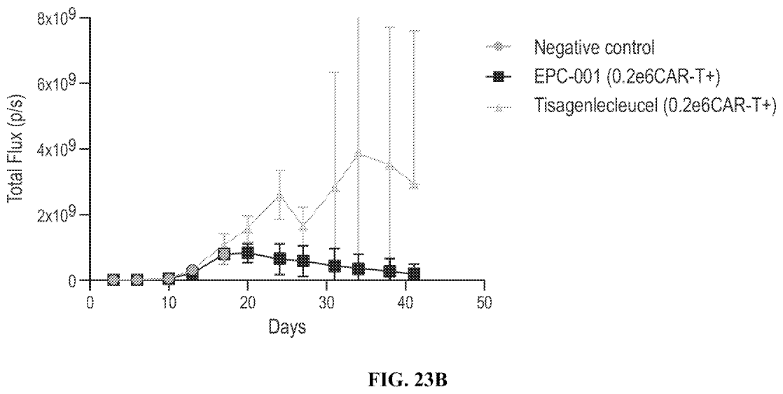

The in vivo cytotoxicity of EPC-001-23 bi-specific CAR-T cells and tisagenlecleucel (as a positive control) were also examined in a NCG mouse model implanted with luciferase-expressing Raji cells and CD19 KO Raji cells. The CAR-T cells were generated as described above. At day 3, PBS control (Group 1), 0.2e6 EPC-001-23 CAR-T cells and tisagenlecleucel CAR-T cells were dosed to the mice implanted with 0.3e6 of parental Raji. The mice were imaged every 3-4 days and body weight were measured. As shown in FIGS. 22A and 22B, bispecific EPC-001-23 CAR-T cells showed stronger and more persistent anti-tumor activity in mice engrafted with parental Raji cells as compared to tisagenlecleucel. FIG. 22B. Similarly, bispecific EPC-001-23 CAR-T cells showed stronger and more persistent anti-tumor activity in mice engrafted with CD19 KO parental Raji cells as compared to tisagenlecleucel. FIGS. 23A and 23B.

The results from this Example confirms the cytotoxicity of the bi-specific CAR-T cells against cells expressing both target antigens and cells expressing only one target antigen. As such, the bi-specific CAR-T cells would be expected to maintain treatment efficacy in the context of target escape, which can be a problem associated with monospecific CAR-T therapy.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A bi-specific chimeric antigen receptor (CAR) specific to CD19 and CD22, comprising a first antigen binding moiety specific to CD19, a second antigen binding moiety to CD22, a co-stimulatory signaling domain, and a cytoplasmic signaling domain;
wherein the first antigen binding moiety comprises the same heavy chain complementary determining regions (CDRs) and the same light chain CDRs as reference antibody EPC-001-1, which binds CD19; and
wherein the second antigen binding moiety comprises the same heavy chain CDRs and the same light chain CDRs as reference antibody EPC-001-2, EPC-001-3, or EPC-001-4, each of which binds CD22.

2. The bi-specific CAR of claim 1, wherein the first antigen binding moiety comprises the same heavy chain variable region (V_{H}) and the same light chain variable region (V_{L}) as the reference antibody EPC-001-1.

3. The bi-specific CAR of claim 1, wherein the second antigen binding moiety comprises the same heavy chain variable region (V_{H}) and the same light chain variable region (V_{L}) as the reference antibody EPC-001-2, EPC-001-3, or EPC-001-4.

4. The bi-specific CAR of claim 1, wherein the first antigen binding moiety, the second antibody binding moiety, or both are single-chain variable fragments (scFvs).

5. The bi-specific CAR of claim 4, wherein the first antigen binding moiety is a scFv comprising the amino acid sequence of SEQ ID NO: 9.

6. The bi-specific CAR of claim 4, wherein the second antigen binding moiety is a scFv comprising the amino acid sequence of SEQ ID NO: 18, 27, or 36.

7. The bi-specific CAR of claim 1, wherein the co-stimulatory signaling domain is from a co-stimulatory molecule selected from the CD28, 4-1BB, OX40, ICOS, CD27, CD40, or CD4OL.

8. The bi-specific CAR of claim 1, wherein the cytoplasmic signaling domain is from CD3.

9. The bi-specific CAR of claim 1, wherein the bi-specific CAR comprises:
(a) a fusion polypeptide comprising, from N-terminus to C-terminus, (i) the first antigen binding moiety, (ii) the second antigen binding moiety, (iii) the co-stimulatory signaling domain, and (iv) the cytoplasmic signaling domain; or
(b) a fusion polypeptide comprising, from N-terminus to C-terminus, (i) the second antigen binding moiety, (ii) the first antigen binding moiety, (iii) the co-stimulatory signaling domain, and (iv) the cytoplasmic signaling domain.

10. The bi-specific CAR of claim 9, further comprising a hinge domain and a transmembrane domain, which are located between (ii) and (iii).

11. The bi-specific CAR of claim 9 or claim 10, further comprising a peptide linker connecting the first antigen binding moiety and the second antigen binding moiety.

12. The bi-specific CAR of claim 11, wherein the peptide linker comprises the amino acid sequence of GGGGS (SEQ ID NO:38), GGGGSGGGGS (SEQ ID NO:39), GGGGSGGGGSGGGGS (SEQ ID NO:40), or GSTSGSGKPGSGEGSTKG (SEQ ID NO:41).

13. The bi-specific CAR of claim 12, which comprises the amino acid sequence of any one of SEQ ID NOs: 48-53.

14. The bi-specific CAR of claim 13, which comprises the amino acid sequence of any one of SEQ ID NOs: 55-60 and 63-67.

15. A nucleic acid or a set of nucleic acid, which collectively encode the bi-specific CAR of claim 1.

16. The nucleic acid or set of nucleic acid of claim 15, which comprises a nucleotide sequence encoding the bi-specific CAR comprising
(a) a fusion polypeptide comprising, from N-terminus to C-terminus, (i) the first antigen binding moiety, (ii) the second antigen binding moiety, (iii) the co-stimulatory signaling domain, and (iv) the cytoplasmic signaling domain; or
(b) a fusion polypeptide comprising, from N-terminus to C-terminus, (i) the second antigen binding moiety, (ii) the first antigen binding moiety, (iii) the co-stimulatory signaling domain, and (iv) the cytoplasmic signaling domain.

17. The nucleic acid or set of nucleic acid of claim 16, which further comprises a nucleotide sequence encoding a truncated epithelium growth factor receptor (EGFR) domain comprising an extracellular domain and a transmembrane domain of an EGFR receptor, and a nucleotide sequence encoding a self-cleaving peptide, which is located between the nucleotide sequence encoding the bi-specific CAR and the nucleotide sequence encoding the truncated EGFR domain.

18. The nucleic acid or set of nucleic acid of claim 17, wherein the truncated EGFR domain comprises the amino acid sequence of SEQ ID NO:68.

19. The nucleic acid or set of nucleic acid of claim 15, wherein the nucleic acid(s) is an expression vector(s), which optionally is a viral vector(s).

20. A genetically engineered immune cell, which expresses the bi-specific CAR of claim 1.

21. The genetically engineered immune cell of claim 20, which comprises the nucleic acid encoding the bi-specific CAR.

22. The genetically engineered immune cell of claim 20, which is a T cell, an NK cell, or a macrophage, optionally wherein the immune cell is a T cell.

23. An anti-CD19 chimeric antigen receptor (CAR), comprising an extracellular antigen binding domain that binds CD19, a co-stimulatory signaling domain, and a cytoplasmic signaling domain; wherein the extracellular antigen binding domain is an anti-CD19 single chain variable fragment (scFv) comprising the same heavy chain complementary determining regions (CDRs) and the same light chain CDRs as anti-CD19 antibody EPC-001-1.

24. The anti-CD19 CAR of claim 23, wherein the anti-CD19 scFv comprises the same heavy chain variable domain and the same light chain variable domain as anti-CD19 antibody EPC-001-1.

25. The anti-CD19 CAR of claim 24, wherein the anti-CD19 scFv comprises the amino acid sequence of SEQ ID NO: 9.

26. The anti-CD19 CAR of claim 25, which comprises the amino acid sequence of SEQ ID NO: 62.

27. An anti-CD22 chimeric antigen receptor (CAR), comprising an extracellular antigen binding domain that binds CD22, a co-stimulatory signaling domain, and a cytoplasmic signaling domain; wherein the extracellular antigen binding domain is an anti-CD22 single chain variable fragment (scFv) comprising the same heavy chain complementary determining regions (CDRs) and the same light chain CDRs as anti-CD22 antibody EPC-001-2, EPC-001-3, or EPC-001-4.

28. The anti-CD22 CAR of claim 27, wherein the anti-CD22 scFv comprises the same heavy chain variable domain and the same light chain variable domain as anti-CD22 antibody EPC-001-2, EPC-001-3, or EPC-001-4.

29. The anti-CD22 CAR of claim 28, wherein the anti-CD22 scFv comprises the amino acid sequence of SEQ ID NO: 18, 27, or 36.

30. The anti-CD22 CAR of claim 29, which comprises the amino acid sequence of SEQ ID NO: 61.

31. A nucleic acid, which encodes the anti-CD19 CAR comprising an extracellular antigen binding domain that binds CD19, a co-stimulatory signaling domain, and a cytoplasmic signaling domain; wherein the extracellular antigen binding domain is an anti-CD19 single chain variable fragment (scFv) comprising the same heavy chain complementary determining regions (CDRs) and the same light chain CDRs as anti-CD19 antibody EPC-001-1 or
encodes the anti-CD22 CAR comprising an extracellular antigen binding domain that binds CD22, a co-stimulatory signaling domain, and a cytoplasmic signaling domain; wherein the extracellular antigen binding domain is an anti-CD22 single chain variable fragment (scFv) comprising the same heavy chain complementary determining regions (CDRs) and the same light chain CDRs as anti-CD22 antibody EPC-001-2, EPC-001-3, or EPC-001-4 .

32. The nucleic acid of claim 31, which is an expression vector, optionally wherein the expression vector is a viral vector.

33. A genetically engineered immune cell, which expresses the anti-CD19 CAR the anti-CD22 CAR set forth in claim 31.

34. The genetically engineered immune cell of claim 33, which is a T cell.

35. A method for eliminating undesired cells in a subject, the method comprising administering to a subject in need thereof an effective amount of the genetically engineered immune cell of claim 18, or a pharmaceutical composition comprising such.

36. The method of claim 35, wherein the undesired cells are cancer cells.

37. The method of claim 35, wherein the subject is a human cancer patient.

38. The method of claim 37, wherein the human cancer patient comprises CD19⁺ and/or CD22⁺ cancer cells.

39. The method of claim 37, wherein the human cancer patient has a hematopoietic malignancy, which optionally is a T cell malignancy or a B cell malignancy.


SEQUENCE LISTING (78 sequences)

SEQ ID NO: 1 - protein, 5 aa, Artificial Sequence
  Synthetic
GYYWT

SEQ ID NO: 2 - protein, 16 aa, Artificial Sequence
  Synthetic
EINHGGSSNYNPSLKS

SEQ ID NO: 3 - protein, 17 aa, Artificial Sequence
  Synthetic
GLGYRSGWYEVENAFDI

SEQ ID NO: 4 - protein, 125 aa, Artificial Sequence
  Synthetic
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSS

SEQ ID NO: 5 - protein, 11 aa, Artificial Sequence
  Synthetic
GGNKIESRSVH

SEQ ID NO: 6 - protein, 7 aa, Artificial Sequence
  Synthetic
DDGARPS

SEQ ID NO: 7 - protein, 9 aa, Artificial Sequence
  Synthetic
QVWDGSSVI

SEQ ID NO: 8 - protein, 106 aa, Artificial Sequence
  Synthetic
QPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVL

SEQ ID NO: 9 - protein, 246 aa, Artificial Sequence
  Synthetic
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVL

SEQ ID NO: 10 - protein, 5 aa, Artificial Sequence
  Synthetic
SYGIS

SEQ ID NO: 11 - protein, 17 aa, Artificial Sequence
  Synthetic
WISAYNGNTNYAQKLQG

SEQ ID NO: 12 - protein, 12 aa, Artificial Sequence
  Synthetic
DPGIAVAGTVDY

SEQ ID NO: 13 - protein, 121 aa, Artificial Sequence
  Synthetic
QVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSS

SEQ ID NO: 14 - protein, 11 aa, Artificial Sequence
  Synthetic
RASQSVSSNLA

SEQ ID NO: 15 - protein, 7 aa, Artificial Sequence
  Synthetic
GASIKAT

SEQ ID NO: 16 - protein, 10 aa, Artificial Sequence
  Synthetic
QQYHTWTPVT

SEQ ID NO: 17 - protein, 108 aa, Artificial Sequence
  Synthetic
EIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIK

SEQ ID NO: 18 - protein, 244 aa, Artificial Sequence
  Synthetic
QVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIK

SEQ ID NO: 19 - protein, 5 aa, Artificial Sequence
  Synthetic
SYGMH

SEQ ID NO: 20 - protein, 17 aa, Artificial Sequence
  Synthetic
VIWYDGSNKYYADSVKG

SEQ ID NO: 21 - protein, 8 aa, Artificial Sequence
  Synthetic
DGWTGFDY

SEQ ID NO: 22 - protein, 117 aa, Artificial Sequence
  Synthetic
EVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSS

SEQ ID NO: 23 - protein, 11 aa, Artificial Sequence
  Synthetic
RASQSVSSNLA

SEQ ID NO: 24 - protein, 7 aa, Artificial Sequence
  Synthetic
GASIKAT

SEQ ID NO: 25 - protein, 10 aa, Artificial Sequence
  Synthetic
QQYHTWPPVT

SEQ ID NO: 26 - protein, 108 aa, Artificial Sequence
  Synthetic
EIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 27 - protein, 240 aa, Artificial Sequence
  Synthetic
EVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 28 - protein, 5 aa, Artificial Sequence
  Synthetic
SYGIS

SEQ ID NO: 29 - protein, 17 aa, Artificial Sequence
  Synthetic
WISAYNGNTNYAQKLQG

SEQ ID NO: 30 - protein, 10 aa, Artificial Sequence
  Synthetic
DYGDPSGDDY

SEQ ID NO: 31 - protein, 119 aa, Artificial Sequence
  Synthetic
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSS

SEQ ID NO: 32 - protein, 11 aa, Artificial Sequence
  Synthetic
RASQSVSSNLA

SEQ ID NO: 33 - protein, 7 aa, Artificial Sequence
  Synthetic
GASIKAT

SEQ ID NO: 34 - protein, 10 aa, Artificial Sequence
  Synthetic
QQYHTWPPVT

SEQ ID NO: 35 - protein, 108 aa, Artificial Sequence; identical to SEQ ID NO: 26
  Synthetic

SEQ ID NO: 36 - protein, 242 aa, Artificial Sequence
  Synthetic
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIK

SEQ ID NO: 37 - protein, 22 aa, Artificial Sequence
  Synthetic
MLLLVTSLLLCELPHPAFLLIP

SEQ ID NO: 38 - protein, 5 aa, Artificial Sequence
  Synthetic
GGGGS

SEQ ID NO: 39 - protein, 10 aa, Artificial Sequence
  Synthetic
GGGGSGGGGS

SEQ ID NO: 40 - protein, 15 aa, Artificial Sequence
  Synthetic
GGGGSGGGGSGGGGS

SEQ ID NO: 41 - protein, 18 aa, Artificial Sequence
  Synthetic
GSTSGSGKPGSGEGSTKG

SEQ ID NO: 42 - protein, 12 aa, Artificial Sequence
  Synthetic
ESKYGPPCPPCP

SEQ ID NO: 43 - protein, 28 aa, Artificial Sequence
  Synthetic
MFWVLVVVGGVLACYSLLVTVAFIIFWV

SEQ ID NO: 44 - protein, 42 aa, Artificial Sequence
  Synthetic
KRGRKKLLYIFKQPFMRPVQTTQEEDGCSCRFPEEEEGGCEL

SEQ ID NO: 45 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 24 aa, Artificial Sequence
  Synthetic
LEGGGEGRGSLLTCGDVEENPGPR

SEQ ID NO: 47 - protein, 357 aa, Artificial Sequence
  Synthetic
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 48 - protein, 526 aa, Artificial Sequence
  Synthetic
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGGGGSGGGGSGGGGSGGGGSQVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIKDYKDDDDKGGHHHHHH

SEQ ID NO: 49 - protein, 524 aa, Artificial Sequence
  Synthetic
QVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIKGSTSGSGKPGSGEGSTKGQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLDYKDDDDKGGHHHHHH

SEQ ID NO: 50 - protein, 522 aa, Artificial Sequence
  Synthetic
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGGGGSGGGGSGGGGSGGGGSEVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKDYKDDDDKGGHHHHHH

SEQ ID NO: 51 - protein, 520 aa, Artificial Sequence
  Synthetic
EVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGSTSGSGKPGSGEGSTKGQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLDYKDDDDKGGHHHHHH

SEQ ID NO: 52 - protein, 524 aa, Artificial Sequence
  Synthetic
QVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGGGGSGGGGSGGGGSGGGGSEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKDYKDDDDKGGHHHHHH

SEQ ID NO: 53 - protein, 522 aa, Artificial Sequence
  Synthetic
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGSTSGSGKPGSGEGSTKGQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLDYKDDDDKGGHHHHHH

SEQ ID NO: 54 - protein, 381 aa, Artificial Sequence
  Synthetic
LEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 55 - protein, 724 aa, Artificial Sequence
  Synthetic
MLLLVTSLLLCELPHPAFLLIPQVQLQQWGAGLLKPSETLSLTCAVYGGSFSGYYWTWIRQPPGKGLEWIGEINHGGSSNYDPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGSTSGSGKPGSGEGSTKGQVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 724 aa, Artificial Sequence
  Synthetic
MLLLVTSLLLCELPHPAFLLIPQVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDPGIAVAGTVDYWGQGTLVTVSSGGGGSGGGGSGGGGSEIVMTQSPATLSVSPGEGVTLSCRASQSVSSNLAWYQQRPGQAPRLLIYGASIKATDVPDRFSGGGSGTDFTLSISNVQSEDFAVYYCQQYHTWTPVTFGGGTKVEIKGSTSGSGKPGSGEGSTKGQVQLQQWGAGLLKPSETLSLTCAVYGGSFSGYYWTWIRQPPGKGLEWIGEINHGGSSNYDPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 720 aa, Artificial Sequence
  Synthetic
MLLLVTSLLLCELPHPAFLLIPQVQLQQWGAGLLKPSETLSLTCAVYGGSFSGYYWTWIRQPPGKGLEWIGEINHGGSSNYDPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGSTSGSGKPGSGEGSTKGEVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 720 aa, Artificial Sequence
  Synthetic
MLLLVTSLLLCELPHPAFLLIPEVQLVQSGGGVVQPGKSLRLSCAASGFTFSSYGMHWVRQAPGKGLEWVAVIWYDGSNKYYADSVKGRFTISRDNSKNTLYLQMNSLRAEDTAVYYCARDGWTGFDYWGQGTTVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGSTSGSGKPGSGEGSTKGQVQLQQWGAGLLKPSETLSLTCAVYGGSFSGYYWTWIRQPPGKGLEWIGEINHGGSSNYDPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 722 aa, Artificial Sequence
  Synthetic
MLLLVTSLLLCELPHPAFLLIPQVQLQQWGAGLLKPSETLSLTCAVYGGSFSGYYWTWIRQPPGKGLEWIGEINHGGSSNYDPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLGSTSGSGKPGSGEGSTKGEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 719 aa, Artificial Sequence
  Synthetic
MLLLVTSLLLCELPHPAFLLIPEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGGGGSGGGGSGGGGSQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 839 aa, Artificial Sequence
  Synthetic
MLLLVTSLLLCELPHPAFLLIPEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 62 - protein, 843 aa, Artificial Sequence
  Synthetic
MLLLVTSLLLCELPHPAFLLIPQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 63 - protein, 709 aa, Artificial Sequence
  Synthetic
MLLLVTSLLLCELPHPAFLLIPEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGGGGSQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 714 aa, Artificial Sequence
  Synthetic
MLLLVTSLLLCELPHPAFLLIPEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGGGGSGGGGSQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 719 aa, Artificial Sequence; identical to SEQ ID NO: 60
  Synthetic

SEQ ID NO: 66 - protein, 722 aa, Artificial Sequence
  Synthetic
MLLLVTSLLLCELPHPAFLLIPEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGSTSGSGKPGSGEGSTKGQVQLQQWGAGLLKPSETLSLTCAVYGGSFSGYYWTWIRQPPGKGLEWIGEINHGGSSNYNPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 709 aa, Artificial Sequence
  Synthetic
MLLLVTSLLLCELPHPAFLLIPEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYGDPSGDDYWGQGTLVTVSSGGGGSGGGGSGGGGSEIVLTQSPATLSVSPGEGVTLSCRASQSVSSNLAWYQQRPGQAPRLLIYGASIKATDVPDRFSGGGSGTDFTLSISNLQSEDFAVYYCQQYHTWPPVTFGGGTKVEIKGGGGSQVQLQQWGAGLLKPSETLSLTCAVYGGSFSGYYWTWIRQPPGKGLEWIGEINHGGSSNYDPSLKSRVTISVDTSKKQFSLNLNSVTAADTAVYYCARGLGYRSGWYEVENAFDIWGQGTMVTVSSGGGGSGGGGSGGGGSQPVLTQPPSVSVAPGQTARITCGGNKIESRSVHWYQQKPGQAPVLVVYDDGARPSGIPERLSGSNSGDTATLTISRVEPGDEADYYCQVWDGSSVIFGGGTKLTVLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 335 aa, Artificial Sequence
  Synthetic
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 69 - DNA, 20 bp, Artificial Sequence
  Synthetic
ctaggtccgaaacattccac

SEQ ID NO: 70 - DNA, 20 bp, Artificial Sequence
  Synthetic
ggaaagtattattgtcaccg

SEQ ID NO: 71 - DNA, 20 bp, Artificial Sequence
  Synthetic
gcaatgacttaggccccttg

SEQ ID NO: 72 - DNA, 20 bp, Artificial Sequence
  Synthetic
aagatgaagaatgcccacaa

SEQ ID NO: 73 - DNA, 20 bp, Artificial Sequence
  Synthetic
atgaaaagccagatggccag

SEQ ID NO: 74 - DNA, 20 bp, Artificial Sequence
  Synthetic
attcataccgggtaacactg

SEQ ID NO: 75 - DNA, 20 bp, Artificial Sequence
  Synthetic
aagactctatgaaagcacaa

SEQ ID NO: 76 - DNA, 20 bp, Artificial Sequence
  Synthetic
ctcttccaacaaattacacg

SEQ ID NO: 77 - DNA, 20 bp, Artificial Sequence
  Synthetic
aaacctgcgcgaagtgacca

SEQ ID NO: 78 - DNA, 20 bp, Artificial Sequence
  Synthetic
ttcccatggtgactccactg